(19) United States
(12) Patent Application Publication
(10) Pub. No.: US 2017/0058348 A1
(43) Pub. Date: Mar. 2, 2017

(54) Predictive mRNA Biomarkers for the Prediction of the Treatment with Methotrexate (MTX)

(71) Applicant: Charité - Universitätsmedizin Berlin, Berlin (DE)

(72) Inventors: Bruno STUHLMÜLLER, Berlin (DE); Karsten MANS, Berlin (DE); Thomas HÄUPL, Erkner (DE); Gerd-R. BURMESTER, Berlin (DE)

(21) Appl. No.: 15/118,711

(22) PCT Filed: Feb. 13, 2015
(86) PCT No.: PCT/EP2015/053095

§ 371 Date: Aug. 12, 2016

(30) Foreign Application Priority Data
Feb. 14, 2014 (DE) 10 2014 101 905.9

(51) Int. Cl. C12Q 1/68

(52) U.S. Cl. CPC C12Q 1/6883; C12Q 2600/106; C12Q 2600/158; C12Q 2600/178

(57) ABSTRACT

The present invention concerns predictive mRNA biomarkers, which are used in combination with HLA-DRB4 for forecasting the treatment with MTX (methotrexate). The present invention further concerns a method for forecasting the treatment with MTX (methotrexate), comprising detection of the predictive mRNA biomarkers in combination with HLA-DRB4 in patient samples, whereby the patients are classified into responders or non-responders.

The present invention relates to predictive mRNA biomarkers, which are used in combination with HLA-DRB4 for forecasting the treatment with MTX (methotrexate). The present invention further relates to a method for forecasting the treatment with MTX (methotrexate), comprising detection of the predictive mRNA biomarkers in combination with HLA-DRB4 in patient samples, wherein the patients are classified into responders or non-responders.

### BACKGROUND OF THE INVENTION

Rheumatoid arthritis (RA) is a chronic inflammatory disease of the musculoskeletal system. Cartilage and bone structures are destroyed with increasing disease progression. Between 0.5% and 3% of the total population, depending on the ethnic group, is affected by RA. Globally, the annual incidence is given as being between 0.1% and 0.5%. In Germany, approximately 2% of the total population (1.5 million) is affected by this disease; annually, this increases by 100,000 new cases. The average costs of this disease, calculated as an average per patient, are calculated at >23,000 [symbol] .

However, to date only little is known as regards the etiology and pathophysiology of RA. The “rheumatoid factor” is a laboratory parameter in the diagnosis of many rheumatic diseases, but only occurs in approximately 60% of RA patients (Meyer et al, 1999). The currently most widely used laboratory test for diagnosing RA is the anti-citrullin (anti-CCP) test, which has a substantially greater specificity than the RF test alone. Both test systems have a very good correlation (van Gaalen et al, 2004; Umeda et al, 2013). On the other hand, correlations with human leukocyte antigens (Heldt et al, 2003) have indicated an increased risk of falling ill with RA.

These disease associations were able to be consolidated on a genetic level, in particular for HLA-DRB1 in connection with the so-called “shared epitope” (O'Dell et al, 1998), or indeed for the expression of the HLA-DRB1 molecule in connection with specific nucleotide exchanges within the lymphotoxin-alpha/TNF-alpha region in patients with early RA (Criswell et al, 2004). The presence of the HLA-DRB1 surface antigen with the increased risk of falling ill with RA only occurs, however, in about 40% of cases and is, according to the current state of knowledge alone, neither indicative for the disease itself nor for a forecast of therapy. Anderson et al. (2000) describe the connection between a reduced therapeutic response in patients with longer disease duration, and Haberauer and Peichl (1989) were able to demonstrate a correlation between the presence of HLA-DRB4 and the rheumatoid factor on a genetic level. Heldt et al. (2003) report, in patients with early RA, a correlation between the simultaneous expression of HLA-DRB1*04 and HLA-DRB4 alleles and an increased radiological progression.

Methotrexate (MTX) is the drug of choice in rheumatoid arthritis and is prescribed for approximately 98% of patients immediately following initial diagnosis. Furthermore, MTX is also used in other autoimmune diseases, and furthermore is currently used as a chemotherapy drug in various tumor diseases (see Abolmaali et al, 2013 and http://www.cancerresearchuk.org/cancer-help/about-cancer/treatment/cancer-drugs/methotrexate).

It is important to carry out effective treatment right from commencement of the disease in order to stop the progress of the disease or to put it into full remission. Unfortunately, treatment with MTX is only 40% to 60% successful and is not infrequently associated with adverse effects (5% to 20%). After the first year of a poor response to therapy, therefore, combination therapies with MTX and biologicas, which increase the rate of responses to therapy to approximately 60% to a maximum of 70%, depending on the biologica used and the status of the individual patient, are now employed. If severe intolerances develop in the patient, the attending physician is allowed to change the therapy applied somewhat sooner. The disease activity in rheumatoid arthritis is determined using the so-called “Disease Activity Score” implemented over n=28 defined joints (DAS28) set by the European League Against Rheumatism (blood sedimentation rate, C-reactive protein, assessment using the Visual Analogue Square (VAS), number of swollen and number of painful joints).

In Germany, according to epidemiological assessments, there are approximately 80,000 patients with RA, all being treated because of chronic inflammation. Annual treatment costs for RA in Germany are >7 billion [symbol] ; globally, they are approximately 180 billion [symbol] . In order to keep costs due to lack of response and adverse effects of the drug for health services and further socioeconomic services as low as possible, individualized therapies are necessary. In this regard, it is necessary to be able to assess the outcome of therapy using biomarkers even in the preliminary stages, so that not only with RA, but also with other autoimmune diseases and tumor therapies, more gentle therapies which are without adverse effects can be specifically applied.

Thus, there is a need in the prior art for predictive test methods for standard drugs which are used in the treatment of rheumatic diseases such as RA, such as MTX in particular, which in addition can be cost-effective and quick to deploy.

The object of the present invention is thus to identify and define suitable biomarkers and to develop suitable test systems in order to provide an improved forecast of the treatment of rheumatic and other diseases.

Predictive mRNA Biomarker Genes

In accordance with the invention, the object is accomplished by means of the use of at least one gene, which is selected from the following 32 genes: ARG1 , CKAP4, CRISP3, CST3, GCLM, KIAA0564, KIAA1324, LCN2, LOC654433/PAX8-AS1, LTF, OLFM4, OSBPL1A, MMP8, SIAH1, SLC8A1/BF223010, SULF2, AQP3, CFD, DEFA4, EIF5A, GATA3, Hs.674648, KCNE3, PAM, PRDX5, RNASE2, TCN1, TKT, SLC35E2, SNHG5, SPAG9, and/or WLS in combination with HLA-DRB4 as predictive biomarkers for forecasting the treatment with MTX (methotrexate)/for predicting the outcome of therapy with MTX.

Preferably, the gene/genes is/are used in the form of its/their mRNA.

The following 16 genes are assigned to the “HLA-DRB4-positive patient group”: ARG1, CKAP4, CRISP3, CST3, GCLM, KIAA0564, KIAA1324, LCN2, LOC654433/PAX8-AS1, LTF, OLFM4, OSBPL1A, MMP8, SIAH1, SLC8A1/BF223010 and SULF2.

The gene descriptions “LOC654433” and “LOC654433/PAX8-AS1” refer to the same gene.

The following further 16 genes are assigned to the “HLA-DRB4-negative patient group”: AQP3, CFD, DEFA4, EIF5A, GATA3, Hs.674648, KCNE3, PAM, PRDX5, RNASE2, TCN1, TKT, SLC35E2, SNHG5, SPAG9 and WLS.

The “HLA-DRB4-positive patient group” and the “HLA-DRB4-negative patient group” refer here to patients in whose samples HLA-DRB4 mRNA is expressed or not expressed as a selection marker.

As an example, HLA-DRB4 mRNA is expressed as a selection marker when a limiting value/cut-off value is reached or exceeded.

As an example, HLA-DRB4 mRNA is not expressed when a limiting value/cut-off value is not reached.

As an example, the cut-off value for HLA-gene expression may be defined as signal values for the HLA-DRB4-negative patient sub-group of ≦100 and of ≧1000 for the HLA-DRB4-positive sub-group. See, Table 4, for example.

A “predictive marker” refers to a marker which provides a prediction of the future course of the response to be expected (here: response and non-response) as regards a drug. The predictive marker provides a forecast of the outcome of a course of treatment with a drug such as MTX even before commencing the treatment. The predictive marker allows this prediction to be made for individual patients.

A “predictive marker” is in particular distinguished from a prognostic marker because for a prognosis, at least 2 measurement points in time are required in order to categorize a patient as a responder or non-responder.

In accordance with the invention, the patients are preferably classified into responders or non-responders.

In the HLA-DRB4-positive patient group, medium responders or moderate responders are counted as responders.

This is the case, for example, for the exemplary embodiment of RA.

In the HLA-DRB4-negative patient group, medium responders or moderate responders are counted as non-responders.

This is the case, for example, for the exemplary embodiment of RA.

Methotrexate (MTX) is the drug of choice in rheumatoid arthritis and is used in approximately 98% of patients immediately following initial diagnosis. Furthermore, MTX is also used in other autoimmune diseases and is, moreover, currently used as a chemotherapy drug in various tumor diseases (see Abolmaali et al, 2013 and http://www.cancerresearchuk.org/cancer-help/about-cancer/treatment/cancer-drugs/methotrexate or http://www.drugs.com/monograph/methotrexate.html#r262).

In one embodiment, the treatment with methotrexate (MTX) includes the combination with biologics and MTX.

The term “biologics” means
anti-TNF antibodies,
   such as, for example, monoclonal anti-TNF antibodies, such as adalimumab (Humira®), certolizumab (Cimzia), golimumab (Simponi®), infliximab (Remicade®).
   (see den Broeder et al, 2002; Barra et al, 2014);
anti-TNF inhibitors,
   such as etanercept (Enbrel®)
   (see Cohen et al, 2008; Rubbert-Roth and Finckh, 2009)
      or
other antibodies,
   such as rituximab (Rituxan®), abatacept (Orencia®), tocilizumab (Actemra® or RoActemra®)
   (see, Taylor 2003, for example).

Preferably, the forecast of the treatment and/or the classification of the patients is carried out prior to starting the treatment with MTX (methotrexate).

In one embodiment, the samples are pre-selected into HLA-DRB4-positive or HLA-DRB4-negative samples.

In accordance with the invention, inflammatory diseases, chronic inflammatory diseases, autoimmune diseases and/or tumor diseases are preferably treated.

In this regard, the inflammatory diseases, chronic inflammatory diseases and autoimmune diseases are preferably selected from:

rheumatoid arthritis (RA) or primary chronic polyarthritis, juvenile idiopathic arthritis, systemic lupus erythematosus (SLE), systemic sclerosis (scleroderma), polymyositis, dermatomyositis, inclusion body myositis, psoriasis, multiple sclerosis, uveitis, Crohn's disease, Churg-Strauss syndrome (CSS), Boeck's disease, Bechterew's disease, relapsing polychondritis, colitis ulcerosa, polymyalgia rheumatica, giant cell arteritis, vasculitis.

In this regard, the tumor diseases are preferably selected from:

acute lymphatic leukemia (ALL) (juvenile and adult), transitional cell carcinoma of the bladder, breast cancer, medulloblastoma, ependymoma (juvenile and adult), non-Hodgkins lymphoma (NHL) (juvenile and adult), osteosarcoma (juvenile and adult).

In a preferred embodiment, at least 50% of the mRNA biomarker genes are assayed in combination with HLA-DRB4.

The term “50% of the biomarker genes” means 16 of the 32 biomarker genes.

In further embodiments, at least 17, 18, 19, 20, 21, 22, 23, 24, 25, 26, 27, 28, 29, 30, 31 or 32 of the 32 biomarker genes are assayed, respectively in combination with HLA-DRB4.

In some embodiments, exclusively the biomarker genes of the HLA-DRB4-positive patient group are assayed; in some embodiments, exclusively the biomarker genes of the HLA-DRB4-negative patient group are assayed; in some embodiments, the biomarker genes of both groups, the HLA-DRB4-positive and the HLA-DRB4-negative patient group, are assayed (respectively in combination with HLA-DRB4).

In the embodiment for the treatment of rheumatoid arthritis (RA), all 32 biomarker genes (i.e. 100%) are assayed in combination with HLA-DRB4.

The use in accordance with the invention preferably comprises assaying the presence of the mRNA marker/biomarker genes and their expression level in a sample.

The presence of the mRNA marker/biomarker genes and the expression level are preferably assayed by means of
sequence-based methods, such as serial analysis of gene expression (SAGE) (such as SuperSAGE), real time quantitative PCR (qPCR) (such as RT-qPCR), bead technology, blot, RNA or next-generation sequencing (such as Ion Torrent),
hybridization-based methods, such as in-situ hybridization, Northern blot, DNA micro- and macroarrays,
   and/or
combinations thereof.

In principle, technologies for research/assaying gene expression can be divided into hybridization-based methods and sequence-based methods.

Examples of hybridization-based methods are:
in the case of in-situ-hybridization, sequence-specific RNA of a defined gene/gene set is detected in tissue and the local gene expression pattern is assayed.
in the Northern blot method, RNA is initially isolated and then separated electrophoretically according to size in a gel. After transferring to a membrane (blotting), the RNA sequence that is being investigated is detected by means of labelled probes (labelled, for example, with radioisotopes, fluorescent dyes) of complementary RNA or DNA via complementary binding. As a rule, only small numbers of sequences are investigated simultaneously.
In DNA microarrays or macroarrays, the quantity of mRNA of a plurality of genes from cells of a culture/tissue can be assayed simultaneously. To this end, the mRNA is isolated and transcribed into cDNA/cRNA. Detection is carried out in this method by means of complementary hybridization of the labelled cDNA/cRNA (labelled, e.g., with radioisotopes, fluorescent dyes) with the probes of the DNA array.

Known DNA microarray techniques use Affymetrix arrays/chips such as, for example, with biotin/streptavidin reinforcement and the dye phycoerythrin.

Examples of sequence-based methods are:
Using serial analysis of gene expression (SAGE), and in particular SuperSAGE, the expression of theoretically all of the genes of a cell can be assayed very precisely, in which a short piece of a sequence (called a “tag”) is produced from each transcript, and as many of these tags as possible are sequenced. The advantage over microarrays is the very precise quantification of the transcripts, as well as the possibility (especially with SuperSAGE) of identifying new transcriptions (for example non-coding ribonucleic acids such as microRNAs or antisense-RNAs) and of investigating organisms with genomes which until now have been unknown.
Real time quantitative PCR (qPCR) is a variation of the polymerase chain reaction (PCR). The concentration of the product is monitored during PCR by means of dyes or special probes added to the reaction mixture. The change in the concentration with time means that conclusions can be drawn as regards the starting concentration of the nucleic acid in question. A special variation of qPCR is reverse-transcriptase real time qPCR (RT-qPCR) and the expanded form, multiplex-qPCR.
RNA sequencing, also known as “next-generation sequencing”, describes assaying the nucleotide sequence of the RNA. To this end, RNA is translated into cDNA so that the DNA sequencing method can be applied. RNA sequencing provides information regarding gene expression, for example how different alleles of a gene are expressed, regarding post-transcriptional modifications or regarding the identification of fusion genes.

The DNA microarray technique measures the relative activity of target genes which have already been identified. Sequence-based methods such as serial analysis of gene expression (SAGE, SuperSAGE), are also used for gene expression analysis. SuperSAGE is particularly precise, since this method is not limited to genes which have already been identified, but any active gene can be measured. Since the introduction of “next generation sequencing methods (RNA seq), sequence-based expression analysis is growing in popularity, because it constitutes a digital alternative to microarrays.

In accordance with the invention, the sample is preferably a patient sample which, more preferably, is selected from whole blood, from peripheral blood leukocytes or from purified blood cells.

In a preferred embodiment, at least one biomarker/gene selected from the following is used: CKAP4, CRISP3, KIAA0564, LCN2, OLFM4, MMP8, or SLC8A1/BF223010 and/or

AQP3, DEFA4, or SNHG5,

in combination with HLA-DRB4 as the predictive biomarker(s) for forecasting the treatment with MTX (methotrexate)/for predicting the therapy response to MTX.

As an example, assaying the presence of the (mRNA) markers and their expression level in a sample is carried out using sequence-based methods, as discussed above, preferably real time quantitative PCR (qPCR) (such as RT-qPCR).

In a preferred embodiment, at least one biomarker/gene selected from:

CRISP3, LCN2, OLFM4 or MMP8 is preferably selected from CKAP4, CRISP3, KIAA0564, LCN2, OLFM4, MMP8, or SLC8A1/BF223010.

Method for Forecasting MTX Treatment

The object is furthermore achieved in accordance with the invention by means of a method for forecasting the treatment with MTX (methotrexate)/for predicting the outcome of therapy with MTX.

The method in accordance with the invention comprises the steps of:

(i) providing a patient sample,

(ii) detecting at least one mRNA biomarker(s) selected from the following 32 genes:
ARG1, CKAP4, CRISP3, CST3, GCLM, KIAA0564, KIAA1324, LCN2, LOC654433/PAX8-AS1, LTF, OLFM4, OSBPL1A, MMP8, SIAH1, SLC8A1/BF223010, SULF2, AQP3, CFD, DEFA4, EIF5A, GATA3, Hs.674648, KCNE3, PAM, PRDX5, RNASE2, TCN1, TKT, SLC35E2, SNHG5, SPAG9, and/or WLS
in combination with HLA-DRB4
in the patient sample,
   and

(iii) assaying the relative expression level of the at least one mRNA biomarker and of HLA-DRB4 by comparison with reference standard(s) and/or control sample(s).

The patients are classified into responders or non-responders using the method in accordance with the invention.

Preferably, the detection in step (ii) comprises assaying the presence of the mRNA marker and its expression level.

Preferably, assaying the relative expression level of the at least one mRNA biomarker and of HLA-DRB4 in step (iii) comprises comparing the expression level with a limiting value or cut-off value.

The limiting value or cut-off value derives from the assay method that is respectively employed, such as arrays, bioplex systems, SAGE, sequencing, qPCR, Multiplex-qPCR etc.

Preferably, assaying the relative expression level of the at least one mRNA biomarker and of HLA-DRB4 in step (iii) further comprises assaying a regulation factor (FC, “fold change”).

As an example, when using Affymetrix arrays/chips, the limiting values or cut-off values are set by the manufacturer of the arrays or chips and/or the assessment software that is used (such as BioRetis, an online database from BioRetis GmbH Berlin).

As an example, the cut-off value for an Affymetrix array/chip may have a signal value of ≧50 and for a BioRetis online database assessment, the amount of regulation factor (FC) is at least 1.5 (|1.5|) in ≧70% of the paired individual comparisons (or within the paired responder versus non-responder group comparisons).

See also Example 1 and Example 2.

In step (iii), expression levels for the at least one mRNA biomarker and for HLA-DRB4 are compared with reference standard(s) and/or control sample(s).

The reference standard(s) in accordance with the invention in step (iii) are preferably sample(s) containing one or more housekeeping gene(s) such as, for example, beta-actin (ACTB), glycerinaldehyde-3-phosphate-dehydrogenase (GAPDH), 60S ribosomal protein P0 (RPLP0).

The control sample(s) in accordance with the invention in step (iii) is (are) preferably samples from responders and/or non-responders.

The control sample(s) in accordance with the invention are preferably reference collectives, i.e. several or a plurality of samples from responders and/or non-responders.

As an example, the 52 patient samples as described herein in the examples are used as the control samples.

The relative expression level of the at least one mRNA biomarker and the present or absent expression of HLA-DRB4 is preferably obtained from a comparison with control sample(s) from responders and/or non-responders.

The inventors have discovered that
for responders compared with non-responders:
   a regulation factor (FC, “fold change”) or a sum of regulation factors of at least 1.5 (or ≧|1.5|)
   is present in at least 70% of the paired individual comparisons (or within the paired responder versus non-responder group comparisons)
   in 100% of the respectively detected mRNA biomarkers,
for non-responders compared with responders, in this regard:
   equivalent thereto, a reciprocal regulation factor or a sum of regulation factors of ≦−1.5
is present in at least 70% of the paired individual comparisons (or within the paired responder versus non-responder group comparisons),
in 100% of the respectively detected mRNA biomarkers.

See also Example 1 (section 1.4) and Example 2.

For the at least 70% of the individual samples/patients it is preferably 60% to 100%, more preferably 70% to 100% or 70% to 90%.

For the paired individual comparisons, each sample or control sample is respectively compared with the individual other samples/control samples.

Preferably, paired individual comparisons are carried out with all of the control samples (i.e. samples from known responders and/or non-responders), in order to carry out the classification into responders and non-responders.

Preferably, the patients are classified as responders when, in step (iii), in 60-100% (preferably at least 70%) of the paired comparisons, the relative expression level reaches a value of ≧|1.5| FC for 100% of the detected mRNA biomarkers.

This means, for example, that if 16 mRNA biomarkers are detected in a sample, for example (in combination with HLA-DRB4), and the expression level of all 16 mRNA biomarkers is over the cut-off value, and the relative expression level is, for example ≧|1.5| in at least 70% (or 60-100%) of the paired comparisons for all 16 markers, then this patient is classified as a responder.

Preferably, the patients are classified as non-responders when, in step (iii), in 60-100% (preferably at least 70%) of the paired comparisons, the relative expression level reaches a reciprocal FC value of ≧|1.5| for 100% of the detected mRNA biomarkers.

This means, for example, that if 16 mRNA biomarkers are detected in a sample, for example (in combination with HLA-DRB4), and the expression level of all 16 mRNA biomarkers is over the cut-off value, and the relative expression level obtained is considered to be ≧|1.5|, for example, in at least 70% (or 60-100%) of the paired comparisons for all 16 markers, then this patient is classified as a non-responder.

In this regard the following 16 genes:

ARG1, CKAP4, CRISP3, CST3, GCLM, KIAA0564, KIAA1324, LCN2, LOC654433/PAX8-AS1, LTF, OLFM4, OSBPL1A, MMP8, SIAH1, SLC8A1/BF223010 and SULF2

are assigned to the “HLA-DRB4-positive patient group”, as described above.

In this regard the following 16 genes:

AQP3, CFD, DEFA4, EIF5A, GATA3, Hs.674648, KCNE3, PAM, PRDX5, RNASE2, TCN1, TKT, SLC35E2, SNHG5, SPAG9 and WLS

are assigned to the “HLA-DRB4-negative patient group”, as described above

In the HLA-DRB4-positive patient group, medium responders or moderate responders are counted as responders.

This is the case, for example, for the exemplary embodiment of RA, as well as for other autoimmune and tumor diseases.

In the HLA-DRB4-negative patient group, medium responders or moderate responders are counted as non-responders.

This is the case, for example, for the exemplary embodiment of RA, as well as for other autoimmune and tumor diseases.

In one embodiment of the method in accordance with the invention, the treatment with methotrexate (MTX) includes the combination with biologics such as, for example, anti-TNF antibodies (as described above), and MTX.

Preferably, the forecast for the treatment and/or the classification of the patients is made before commencing the treatment with MTX (methotrexate).

In a preferred embodiment of the method in accordance with the invention, the sample(s) is/are pre-selected into HLA-DRB4-positive or HLA-DRB4-negative sample(s).

In a preferred embodiment of the method in accordance with the invention, the sample undergoes a pre-treatment.

A pre-treatment of this type may comprise:
removing globin mRNA,
reverse transcription of the total mRNA
   and/or
labelling with a label such as biotin, for example.

Preferably the detection in step (ii) comprises assaying the presence of the mRNA marker and its expression level.

The assay is preferably carried out by means of
sequence-based methods, such as serial analysis of gene expression (SAGE) (such as SuperSAGE), real time quantitative PCR (qPCR) (such as RT-qPCR), bead technology, blot, RNA or next-generation sequencing (such as Ion Torrent),
hybridization-based methods, such as in-situ hybridization, Northern blot, DNA micro- and macroarrays,
   and/or
combinations thereof
   as described above.

In a preferred embodiment of the method in accordance with the invention, in step (ii) at least one mRNA biomarker selected from
CKAP4, CRISP3, KIAA0564, LCN2, OLFM4, MMP8, or SLC8A1/BF223010
and/or
AQP3, DEFA4, or SNHG5
   in combination with HLA-DRB4 is detected in the patient sample.

In a preferred embodiment, at least one mRNA biomarker selected from:

CRISP3, LCN2, OLFM4 or MMP8 is preferably selected from CKAP4, CRISP3, KIAA0564, LCN2, OLFM4, MMP8, or SLC8A1/BF223010.

In accordance with the invention, inflammatory diseases, chronic inflammatory diseases, autoimmune diseases and/or tumor diseases are preferably treated.

In this regard, the inflammatory diseases, chronic inflammatory diseases and autoimmune diseases are preferably selected from:

rheumatoid arthritis (RA) or primary chronic polyarthritis, juvenile idiopathic arthritis, systemic lupus erythematosus (SLE), systemic sclerosis (scleroderma), polymyositis, dermatomyositis, inclusion body myositis, psoriasis, multiple sclerosis, uveitis, Crohn's disease, Churg-Strauss syndrome (CSS), Boeck's disease, Bechterew's disease, relapsing polychondritis, colitis ulcerosa, polymyalgia rheumatica, giant cell arteritis, vasculitis.

In this regard, the tumor diseases are preferably selected from:

acute lymphatic leukemia (ALL) (juvenile and adult), transitional cell carcinoma of the bladder, breast cancer, medulloblastoma, ependymoma (juvenile and adult), non-Hodgkins lymphoma (NHL) (juvenile and adult), osteosarcoma (juvenile and adult).

In a preferred embodiment of the method in accordance with the invention, at least 50% of the biomarker genes are assayed in combination with HLA-DRB4.

“50% of the biomarker genes” means 16 of the 32 biomarker genes.

In further embodiments, at least 17, 18, 19, 20, 21, 22, 23, 24, 25, 26, 27, 28, 29, 30, 31 or 32 of the 32 biomarker genes are assayed, respectively in combination with HLA-DRB4.

In some embodiments, exclusively the biomarker genes of the HLA-DRB4-positive patient group are assayed; in some embodiments, exclusively the biomarker genes of the HLA-DRB4-negative patient group are assayed; in some embodiments, the biomarker genes of both groups, the HLA-DRB4-positive and the HLA-DRB4-negative patient group, are assayed (respectively in combination with HLA-DRB4).

In the embodiment of the method in accordance with the invention for the treatment of rheumatoid arthritis (RA), all 32 biomarker genes (i.e. 100%) are assayed in combination with HLA-DRB4.

In accordance with the invention, the sample is preferably a patient sample, more preferably selected from whole blood, from peripheral blood leukocytes or from purified blood cells.

Kits for Forecasting Treatment with MTX

The object of the invention is further achieved by means of kits for forecasting the treatment with MTX (methotrexate)/for predicting the outcome of therapy with MTX.

A kit in accordance with the invention comprises:
(a) implementing means for detecting at least one mRNA biomarker(s) selected from ARG1, CKAP4, CRISP3, CST3, GCLM, KIAA0564, KIAA1324, LCN2, LOC654433/PAX8-AS1, LTF, OLFM4, OSBPL1A, MMP8, SIAH1, SLC8A1/BF223010, or SULF2 and/or AQP3, CFD, DEFA4, EIF5A, GATA3, Hs.674648, KCNE3, PAM, PRDX5, RNASE2, TCN1, TKT, SLC35E2, SNHG5, SPAG9, or WLS in combination with HLA-DRB4 in patient samples,
(b) reference standard(s) comprising sample(s) containing one or more housekeeping gene(s),
(c) control sample(s) comprising sample(s) from responders and/or non-responders.

Suitable reference standard(s) and control sample(s) are as described above.

In one embodiment of the kit in accordance with the invention, the implementing means for detecting at least one mRNA biomarker(s) are selected from
CKAP4, CRISP3, KIAA0564, LCN2, OLFM4, MMP8, or SLC8A1/BF223010
and/or
AQP3, DEFA4, or SNHG5.

In a preferred embodiment, at least one mRNA biomarker selected from: CRISP3, LCN2, OLFM4 or MMP8 is preferably selected from CKAP4, CRISP3, KIAA0564, LCN2, OLFM4, MMP8, or SLC8A1/BF223010.

The implementing means for detecting at least one mRNA biomarker(s) in patient samples preferably comprise:
arrays, chips (as hereinabove described),
primers,
markers and labels (as hereinabove described),
and/or combinations thereof.

Predictive miRNA Biomarkers

The object of the invention is furthermore achieved by means of the use of at least one miRNA, which is selected from the following 6 miRNAs:

Hsa-mir-193b_st, Hsa-mir-223_st, Hsa-mir-572_st, Hsa-mir-1184, Hsa-mir-1915_st, Hsa-mir-3177_st, and/or Hsa-mir-4298_st

as predictive miRNA biomarkers for forecasting the treatment with MTX (methotrexate)/for predicting the outcome of therapy with MTX.

In accordance with the invention, the patients are preferably classified into responders or non-responders.

As discussed above, methotrexate (MTX) is the drug of choice for rheumatoid arthritis and is prescribed for approximately 98% of patients immediately following initial diagnosis. Furthermore, MTX is also used for other autoimmune diseases and is also a routine drug in chemotherapy for various tumor diseases (see Abolmaali et al, 2013 and http://www.cancerresearchuk.org/cancer-help/about-cancer/treatment/cancer-drugs/methotrexate or http://www.drugs.com/monograph/methotrexate.html#r262).

In one embodiment, the treatment with methotrexate (MTX) includes the combination with biologics and MTX.

The term “biologics” means
anti-INF antibodies,
   such as, for example, monoclonal anti-TNF antibodies, such as adalimumab (Humira®), certolizumab (Cimzia), golimumab (Simponi®), infliximab (Remicade®).
   (see den Broeder et al, 2002; Barra et al, 2014);
anti-TNF inhibitors,
   such as etanercept (Enbrel®)
   (see Cohen et al, 2008; Rubbert-Roth and Finckh, 2009)
      or
other antibodies,
   such as rituximab (Rituxan®), abatacept (Orencia®), tocilizumab (Actemra® or RoActemra®)
   (see Taylor 2003, for example),
      as already discussed above.

Preferably, the forecast of the treatment and/or the classification of the patients is carried out prior to commencing treatment with MTX (methotrexate).

In one embodiment, the samples are pre-selected into HLA-DRB4-positive or HLA-DRB4-negative samples.

In accordance with the invention, inflammatory diseases, chronic inflammatory diseases, autoimmune diseases and/or tumor diseases are preferably treated.

In this regard, the inflammatory diseases, chronic inflammatory diseases and autoimmune diseases are preferably selected from:

rheumatoid arthritis (RA) or primary chronic polyarthritis, juvenile idiopathic arthritis, systemic lupus erythematosus (SLE), systemic sclerosis (scleroderma), polymyositis, dermatomyositis, inclusion body myositis, psoriasis, multiple sclerosis, uveitis, Crohn's disease, Churg-Strauss syndrome (CSS), Boeck's disease, Bechterew's disease, relapsing polychondritis, colitis ulcerosa, polymyalgia rheumatica, giant cell arteritis, vasculitis.

In this regard, the tumor diseases are preferably selected from:

acute lymphatic leukemia (ALL) (juvenile and adult), transitional cell carcinoma of the bladder, breast cancer, medulloblastoma, ependymoma (juvenile and adult), non-Hodgkins lymphoma (NHL) (juvenile and adult), osteosarcoma (juvenile and adult).

The use in accordance with the invention preferably comprises assaying the presence of the miRNA marker(s) in a sample.

The presence of the miRNA marker/biomarkers is preferably assayed using:
sequence-based methods, such as serial analysis of gene expression (SAGE) (such as SuperSAGE), real time quantitative PCR (qPCR) (such as RT-qPCR), bead technology, blot, RNA or next-generation sequencing (e.g. Ion Torrent),
hybridization-based methods, such as in-situ hybridization, Northern blot, DNA micro- and macroarrays,
   and/or
combinations thereof
   as described above.

Preferably, microarray analysis, quantitative PCR and/or bead-based methods are used to assay the miRNAs.

In accordance with the invention, the sample is preferably a patient sample, more preferably selected from whole blood, from peripheral blood leukocytes or from purified blood cells.

Method for Forecasting the MTX Treatment Using miRNA Biomarker(s)

The object of the invention is further achieved by means of a method for forecasting the treatment with MTX (methotrexate)/for predicting the outcome of therapy with MTX.

The method in accordance with the invention comprises the steps of:
(i) providing a patient sample,
(ii) detecting at least one miRNA selected from Hsa-mir-193b_st, Hsa-mir-223_st, Hsa-mir-572_st, Hsa-mir-1184, Hsa-mir-1915_st, Hsa-mir-3177_st, and/or Hsa-mir-4298_st,
   and
(iii) optionally, comparing the detection of the at least one miRNA Biomarkers with a reference standard and/or a control sample.

Preferably, the detection in step (ii) comprises assaying the presence of the miRNA marker.

The patients are classified into responders or non-responders by means of the method in accordance with the invention.

Preferably, the patients are classified as responders when the expression differs from non-responders by a FC value of at least |1.3| and a significance of p=≦0.05 occurs within the comparison with the non-responders.

Preferably, the patients are classified as non-responders when the expression differs from non-responders by a FC value of at least |1.3| and a significance of p=≦0.05 occurs within the comparison with the responders.

In one embodiment of the method in accordance with the invention, the treatment with methotrexate (MTX) includes the combination with biologics such as, for example, anti-TNF antibodies (as described above), and MTX.

Preferably, the forecast of the treatment and/or the classification of the patients is carried out prior to commencing treatment with MTX (methotrexate).

In a preferred embodiment of the method in accordance with the invention, the sample undergoes a pre-treatment.

A pre-treatment of this type may comprise:
removing globin mRNA,
reverse transcription of the total mRNA
   and/or
labelling with indirect labels such as, biotin, streptavidin, for example, and/or
direct labelling with fluorescent dyes.

Preferably, the detection in step (ii) comprises assaying the presence of the miRNA marker.

The assay is preferably carried out using
sequence-based methods, such as serial analysis of gene expression (SAGE) (such as SuperSAGE), real time quantitative PCR (qPCR) (such as RT-qPCR), bead technology, blot, RNA or next-generation sequencing (e.g. Ion Torrent),
hybridization-based methods, such as in-situ hybridization, Northern blot, DNA micro- and macroarrays,
   and/or
combinations thereof
   as described above.

Preferably, microarray analysis and/or quantitative PCR is/are used to assay the miRNAs.

In accordance with the invention, inflammatory diseases, chronic inflammatory diseases, autoimmune diseases and/or tumor diseases are preferably treated.

In this regard, the inflammatory diseases, chronic inflammatory diseases and autoimmune diseases are preferably selected from:

rheumatoid arthritis (RA) or primary chronic polyarthritis, juvenile idiopathic arthritis, systemic lupus erythematosus (SLE), systemic sclerosis (scleroderma), polymyositis, dermatomyositis, inclusion body myositis, psoriasis, multiple sclerosis, uveitis, Crohn's disease, Churg-Strauss syndrome (CSS), Boeck's disease, Bechterew's disease, relapsing polychondritis, colitis ulcerosa, polymyalgia rheumatica, giant cell arteritis, vasculitis.

In this regard, the tumor diseases are preferably selected from:

acute lymphatic leukemia (ALL) (juvenile and adult), transitional cell carcinoma of the bladder, breast cancer, medulloblastoma, ependymoma (juvenile and adult), non-Hodgkins lymphoma (NHL) (juvenile and adult), osteosarcoma (juvenile and adult).

The sample in accordance with the invention is preferably a patient sample, more preferably selected from whole blood, from peripheral blood leukocytes or from purified blood cells. In accordance with the invention, the reference standard/the control sample in step (iv) is preferably a reference standard consisting of housekeeping gene(s), and/or a mixture of control samples from responders and non-responders.

Rheumatoid Arthritis (RA) Embodiment

RA is usually treated with Disease-Modifying Antirheumatic Drugs (DMARDs) immediately following diagnosis by a rheumatologist. The conventionally used MTX falls into this category of drug and is the DMARD of choice in >95% of cases. Success of the therapy has until now not been predictable, and until now has been evaluated by means of the joint destruction that can be detected. Furthermore, MTX is also used for the treatment of other rheumatic diseases, other autoimmune diseases and for the treatment of tumor diseases.

Counting from the commencement of treatment, the individual RA patient exhibits initial benefits after 3-6 weeks, but this is only documented following assessment of the clinical parameters for the determination of the DAS28. What is guaranteed is an assessment of the response rate, but only after 12-14 weeks (Quinn et al, 2005), and the maximum is only obtained after approximately 6 months. Frequently, effectiveness is lost after just a year, when sustained success rates of 40-50% have been observed (Furst 1996), prompting combining MTX therapy with other DMARDs such as sulfasalazine or leflunomide (Smolen et al. 2010), If no improvement is seen here as well, the combination therapy with MTX and biologics is carried out. Assessments of the outcomes of therapy with MTX were investigated by Anderson et al (2000) with the aid of 14 different clinical studies. These comparisons showed that RA patients with a long duration of disease have a poorer response profile than patients in whom the duration of disease is less than one year. A connection between women, who are affected twice as frequently, and a possible forecast of the response to therapy, could not be demonstrated there. Similarly, until now no links between routinely measured clinical parameters, laboratory parameters or environmental results and the forecast of the response to therapy with MTX have been established. Experience has shown that only 40-45% of RA patients respond to a combination therapy with MTX and monoclonal anti-TNF antibodies (see, for example, den Broeder et al. 2002), whereas from a clinical standpoint, the remaining 55-60% of patients do not exhibit the desired therapeutic success. The improvement rates for assessment are acquired in accordance with defined rules which were established by the American College of Rheumatology (ACR response), or rules established by the European League Against Rheumatism (EULAR response). In addition, following a loss of response (intolerance) or the appearance of adverse effects as regards treatment with MTX, further treatment is carried out using other anti-TNF inhibitors (Cohen et al, 2008; Rubbert-Roth and Finckh, 2009) or with other biologics, e.g. with rituximab, abatacept, tocilizumab, certolizumab (see Taylor 2003, for example) or certainly in the future with other therapeutics using the “trial and error” principle (Blom et al, 2009). What is clear is that a course of therapy should be successful and also be applied as early as possible in order to counter any chronicization with progressive joint destruction (Smolen et al, 2010).

The individual responses to therapy have until now not been forecastable. Thus, it would be desirable to define biomarkers and to develop test systems for each individual standard drug which is used in RA.

In the last 10 years, both genetic (SNP analysis) and also genomic (mRNA) test methods have attempted to provide a prediction as regards therapy. The latest approaches have been reported in the present patent application. Genetic investigations have not so far been able to assign statistical significance to singular nucleotide exchanges in individual genes. This is expressed in very low odds ratios. As an example, genetic association approaches in RA patients and carriers of the HLA-DRB1*04:01 provided odds ratio values of only 4.44 (Scally et al. 2013), a value which cannot be assigned any significant statistical relevance. For this reason, the sequencing methods used so far have indicated low correlations for risk management of the disease and for the assessment of forecasts, either for the disease itself or for the response to therapy.

More hope is being placed in the future on the more complex investigations of genome-wide DNA sequencing, which should allow a large number of genes in larger patient collectives to be investigated using very comprehensive bioinformatics algorithms. A connection of the genome-wide transcription analysis with novel types of bulk sequencing approaches is very promising. In summary, it is growing ever clearer that inflammatory chronic diseases and tumor diseases are diseases which have a multifactorial origin and are dependent on sub-groups, and thus it is usually necessary to combine several approaches.

In the present invention, this is exhibited by pre-selection of the HLA-DRB4 sub-groups in combination with the respective 16 specific candidate genes for the assessment of responders and non-responders.

Whole-genome transcription analyses are new technologies which on the one hand allow rapid and adapted transfers into other molecular technologies, and are of very great significance for individualized medicine. Transformation into cost-effective test systems such as, for example, the quantitative polymerase chain reaction, and in particular to “microfluidics” based techniques, can/will contribute to relieve health services from annually increasing costs. Technically, since these methods use whole blood which is routinely taken during clinical investigations as the starting material, they are exceptionally well placed to provide fast and differentiated results to allow the physician, in the context of avoiding adverse effects (>5%), to provide an effective choice of therapy for the individual patient as early as possible.

Until now, there have been no test methods either for MTX or for biologics, which could be cost effective, without complicated logistics and also which could be applied quickly.

The inventors have now succeeded in developing a predictive test for assessing the future response to MTX therapy, in which the following predictive biomarker genes are used in combination with HLA-DRB4:

Predictive Genes from the HLA-DRB4-Negative Sub-Group

Here, the predictive biomarker genes for MTX of the HLA-DRB4-negative RA sub-group in accordance with the invention are set out and discussed:

1. Defensin Alpha 4 (DEFA4; a.k.a.: corticostatin), which is strongly expressed in MIX responders in the HLA-DRB4-negative sub-group, has a multitude of biological functions. DEF4A is described as functioning as a pathogen defense for peptides with a microbial, cytotoxic, antiviral function (Spitznagel, 1990; Wu et al, 2005). Furthermore, DEF4A inhibits the corticotropin-stimulated corticosteroid production (Ganz et al, 1990). DEF3A has been described by Cheok et al. (2003), inter alia, as a marker for discriminating drug responses. These findings were obtained using human leukemia cell lines in vitro.

2. The predictive marker Complement Factor-D (CFD; a.k.a.: adipsin), which is highly regulated in responders of the HLA-DRB4-negative sub-group, functionally belongs to the trypsin family of the peptidases. CFD is a component of the alternative complement pathways and is also involved in the humoral response to defend against infectious pathogens (Jouvin et al, 1983). 3. Transcobalamin-1 (TCN1) codes for a vitamin B12-binding protein and transfers cobalamin into cells. Diseases that are cited in the literature in connection with this gene are pernicious anemia and oral tumors. Interestingly from a genetic viewpoint, polymorphisms have been described within the TCN family which have an influence on MTX metabolism (Linnebank et al. 2005). Parallels between genetic and genomic findings are as yet unknown.

4. Ribonuclease-2 (RNASE2) belongs to the ribonuclease type A family, has ribonuclease activity, hence its name, and binds nucleic acids. It is further specified that RNASE2 is a pyrimidin-specific nuclease which also has a poor binding affinity for uridine, cytotoxin and helminthotoxin. RNASE2 has a further biological role in the immune response and in anti-parasitic defense (Yang et al, 2003; Yang et al, 2004). RNASE2 is also chemotactic for dendritic cells and is an endogenic ligand for the toll-like receptor-2 (Rosenberg 2008).

5. Transketolase-Like 1 (TKTL1) has a functional role in the pentosephosphate pathway and has been described as regulating the action of MTX (Lee et al, 2008). In their investigations, the predictive marker TKT, but not TKTL1, were identified, which is described as a MTX predictor in the rat tumor model (Yamashita et al, 1999).

6. The function of Hs.674648 is as yet unknown, as is also—

7. Peptidylglycine Alpha-amidating Monooxygenase (PAM), a coding enzyme which can bind bivalent copper and calcium ions, is involved in a plurality of different biological functions (Prigge et al, 2000). An indirect or direct connection with MTX interaction with influence on the efficiency is as yet unknown.

8. The potassium channel KCNE3 belongs to the isk-related family. The biological function of potassium channels is manifold. What is known is that with the gene family member 4 (KCNE4) in the rat model, Lee et al. (2008) showed that MTX has an influence on its expression level.

9. Sperm Associated Antigen-9 (SPAG9) mRNA codes for a protein from the tumor-testis antigen family (Garg et al, 2007). The coding protein of SPAG9 mRNA has scaffold protein properties and organizes itself structurally with mitogen-activating protein kinases and thus contributes to c-jun terminal kinase-mediated signal transduction. SPAG9 binds to kinesin-1 and plays a role in tumor growth and development. Until now, there have been no links to MTX.

10. Mitochondrial Precursor Peroxiredoxin-5 (PRDX5) interacts with the peroxisome receptor 1 and has antioxidant protective functions in normal and inflamed tissue (Yamashita et al, 1999). Here again, there is no known connection with MTX.

11. Aquaporin-3 (AQP3) mRNA is under-regulated and codes for a water channel protein (Ishibashi et al, 1995) which, like

12. Wntless Wnt Ligand Secretion Mediator (WLS), is largely unknown as yet as regards its function. Participation of the protein in the NFkB and MAP kinase pathway has been discussed (Matsuda et al, 2003). A direct or indirect connection to MTX is as yet unknown, either for AQP3 or for WLS.

13. The coded GATA-binding Protein-3 (GATA3) carries two GATA-type-specific zinc fingers and is involved in the regulation of T cells in what is known as the “innate lymphoid group 3” (Yagi et al, 2011; Serafini et al, 2014) and in endothelial cell maturation (Umetani et al, 2001). GATA3 apparently has an immunosuppressive and anti-inflammatory action (Li et al, 2013). GATA3 has been described in vitro as a predictor for cytorabine hydrochloride (Ara-C), dexamethasone, methylprednisolone, mitoxantrone and rituximab treatment in tumor cell lines (US 2009/0023149 A1). Furthermore, GATA3 has been described as being a predictor of taxane insensitivity (Tominaga et al, 2012). The mRNA from GATA3 is highly regulated in liver tumor tissue from rats and in human breast cancer cell tissue by treatment with MTX (Belisnky et al, 2007; Gulbahce et al, 2013). However, as yet, a prediction as regards the effectiveness of MTX has not yet been provided from these findings.

14. Eukaryotic Translation Initiation Factor 5A (EIF5A) codes for an mRNA-binding protein which is involved in translation-elongation. It is also known that EIF5A plays a role in methionine metabolism and in hypusine biosynthesis (Scuoppo et al, 2012). The over-expression of EIF5A mRNA in colorectal tumor tissue samples correlates with the level of tumor expression in patients with colorectal cancer diseases. EIF5A was thus proposed as a prognostic marker for the assessment of the success of MTX-treated patients with colorectal cancer diseases (Tunca et al, 2013 and Rat Genome Database, Bioinformatics Research Centre, Medical College of Wisconsin of the National Heart Lung and Blood Institutes (NHLBI)).

15. The mRNA for ‘Solute Carrier Family 35 Member E2’ (SLC35E2) is a new member of the “solute carrier family” and controls sugar transport in the nucleotide. In the model system, it has been shown that this transporter is involved in tumor metastasis, cellular immunity, organogenesis and morphogenesis and in the development of connective tissue and muscle (Ishida & Kawakita 2004). SLC35, and also the other members of this gene family, also have transporter functions, in addition to nucleotide sugar metabolism functions, of drugs inter alia, and are located in the Golgi apparatus and in the endoplasmic reticulum (Nishimura et al, 2009). Pathologically, animal experimental studies employing a deficiency of this gene have indicated increased tumor metastasis, along with a perturbance to immunity, organogenesis and morphogenesis (Ishida & Kawakita 2004).

Genes from the SLC family have been assigned a role in the pharmacokinetics of drugs (WO 2011/014721 A2). In tumor patients who have been treated with tamoxifen, there have been indications that the expression of SLC35E2 changes (Han et al, 2006). The gene expression of many members of the SLC family is altered by methotrexate (see also the Rat Genome Database, Bioinformatics Research Centre, Medical College of Wisconsin of the National Heart Lung and Blood Institutes (NHLBI)).

16. The highly regulated mRNA of the ‘Small Nucleolar RNA Host Gene 5 (SNHG5; a.k.a.: U50HG) is involved in ribosome biogenesis (Tanaka et al, 2000). SNHG5 has been described as a biomarker in B-cell lymphoma, breast and prostate tumors (Dong et al, 2009; Nakamura et al, 2008; Dong et al, 2008) and is strongly expressed therein. Irradiation of tumor cells results in counter-regulation with reduction of the mRNA expression of SNHG5 (Chaudry 2013).

Predictive Genes of the HLA-DRB4-Positive Sub-Group

Here, the biomarker genes of the HLA-DRB4-positive RA sub-group predictive for MTX will be listed and explained:

1. KIAA1324 is a gene with a function which is as yet unknown. KIAA1324 is over-expressed in bowel tumor cells and has been described as a diagnostic marker for epithelial bowel tumors (US 2008/0064049 A1).

2. The gene for E3-ubiquitin protein ligase-1 (SIAH1) codes for a protein from the “seven in absentia homolog” family (Hu et al, 1997; Nakayama et al, 2004). SIAH1 plays a decisive role in the development of various Parkinson's diseases (Franck et al, 2006). SIAH5 is regulated in cooperation with high-density lipoproteins following hypoxia and induction of apoptosis via the jun kinase pathway (Nakayama et al, 2004).

3. Cystatin-3 (CST3; a.k.a.: cystatin-C) codes for a protein which contains many cystatin-like sequence regions (Turk et al, 2008). CST3 is expressed strongly in arteriosclerosis (Arpegard et al, 2008), but also in diseases from the rheumatoid spectrum (Hansen et al, 2000). Hayashi et al. (2010) were able to show that enhanced seric Cys-C is an indicator for MTX-induced myelotoxicity in patients with RA. Like the inventors' findings as regards mRNA, in particular in the MTX responders, CST3 is also highly regulated in RA patients on a protein level even before treatment with MTX. It can be deduced from this that a stronger myelotoxicity should also be anticipated in responders.

4. Sulfatase-2 (SULF2) is a heparan sulfate 6-O-endosulfatase. SULF2 modulates the change in binding sites at cell-signal receptors, via the binding of heparan sulfate (Dai et al. 2005). Increased rates of expression of SULF1 and SULF2 are described both for tumor tissue (Wigersma et al, 1991; Nawroth et al, 2007), and also for inflammatory diseases such as osteoarthritis, for example (Otsuki et al, 2008) or for RA in synovial tissues (Kar et al, 1976).

5. The function of KIAA0564 (a.k.a.: von Willebrand Factor A domain containing 8) is still unknown. However, the description and other indications hint that the von Willebrand Factor A domain containing 8/KIAA0564 is a protein with cell adhesion properties (Reininger et al. 2006). GO annotations show that this protein comprises an ATPase and ATP-binding activity. KIAA0564 has been described in the context of diagnosis and prevention having regard to prediction of therapies (WO 2002/008423 A2).

6. “Glutamate-cysteine Ligase Modifier” (GCLM; a.k.a.: gamma-glutamylcysteine synthetase) is involved in glutathione synthesis. GCLM is important as regards the survivability of erythrocytes (Foller et al, 2013) and is highly regulated in hemolytic anemia. In the MTX responders, GCLM is under-regulated compared with the non-responders of the HLA-DRB4-positive patients sub-group.

7. “Cytoskeleton-Associated Protein 4” (CKAP4) is a transmembrane protein and is expressed in the endoplasmic reticulum. An enhanced expression of CKAP4 was observed in metastasizing lymphatic tissue (Li et al, 2013). Functionally, CKAP4 regulates the plasminogen-activating system of the blood vessels (Razzaq et al, 2003). In addition, a susceptibility for MTX has been reported for CKAP4 (Prigge et al, 2000) and CKAP4 has been described as a predictor of MTX for tumor diseases (U.S. Pat. No. 8,445,198 B2; US 2008/0292546 A1).

8. Oxysterol Binding Protein-Like 1A (OSBPL1A) is co-localized together with the GTPases Rab7, Rab9 and the “Lysosome-associated membrane protein-1” and binds phosphoinositides in endosomes and lysosomes (Johansson et al, 2005). A link to MTX was not described.

9. The expressed gene ‘Solute carrier family 8A member l’ (SLC8A1; a.k.a. BF223010) acts as a sodium/calcium exchanger (Khananshvili, 2013) and GO annotations have indicated that it is a cytoskeletal protein with calmodulin-binding function. The transcriptional regulator (miRNA) of the SLC8A1, but not SLC8A1 itself, has been described as a predictor for MTX treatment in inflammatory bowel diseases, (WO 2009/120877 A2; WO 2011/014721 A2). Various members of the SLC family interact with MTX and are regulated through MTX (see also SLC35E2) (see also the Rat Genome Database, Bioinformatics Research Centre, Medical College of Wisconsin of the National Heart Lung and Blood Institutes (NHLBI).

SLC8A1 has been described as a diagnostic marker for autoimmune diseases, such as systemic lupus erythematosus (SLE), and in ANCA positive wegener granulomatosis (WO 2006/020899 A2).

10. The biomarker LOC654433 is a long non-coding RNA with a function which is as yet unknown.

11. Arginase 1 (ARG1) is a type-I specific arginase, which catalyzes the hydrolysis of arginine to ornithine, with urea being cleaved off (Ivanenkov et al, 2014). Monocytes/macrophages are the principal cell populations, which express arginases (Murphy et al, 1998). Huang et al. (2001) report that arginase activity is significantly associated with arginase protein expression in patients with RA. The gene expression of ARG1 is strengthened in the HLA-DR134-positive sub-group for the MTX responders. Shen et al. (2013) demonstrated a connection of expressions of ARG1 and folate receptor-β on positive M1-type macrophages which also express the mannose receptor. A direct connection between the gene expression of ARG1 and MTX has not been found as yet.

12. Lipocalin 2 (LCN2) is expressed on neutrophils and is associated with the proteolytic enzyme gelatinase (Kjeldsen et al, 2000).

LCN2 is an iron-trafficking protein that is involved in multiple processes such as innate immunity (Zughaier et al, 2013; Landro et al, 2008), renal development, and cell migration (Paulsson et al, 2007). Blaser et al. (1995) reported that lipocalin 2 can be detected in large quantities in synovial fluid from patients with RA. In responders of the HLA-DRB4-positive sub-group, the mRNA coding for this enzyme is reduced.

13. The biological function of the biomarker ‘Cysteine-Rich Secretory Protein 3’ (CRISP3) has not as yet been described. The C-type Lektin Domain Family 18, Member B (CLEC18B) constitutes a paralogue of CRISP3, which—using GO annotation—has the capability of binding carbohydrates, as “mannose receptor-like protein”. CRISP3 interacts with 17 beta-estradiol (Pfisterer et al, 1996). The gene expression of CRISP3 is strongly expressed in DHEA-stimulated human submandibular gland cells (Laine et al, 2007). The gene expression of CRISP3 has been described in connection with the disease known as Sjögren's syndrome (Tapinos et al, 2002). CRISP3 has been described as a predictor for the therapy of prostate cancer cells (WO 2013/070088 A1).

14. Lactotransferrin (LTF; a.k.a.: lactoferrin) is a member of the transferrin gene family and is essentially expressed by neutrophils. The LTF protein has heparin-binding activity and has a broad functional spectrum. This includes, inter alia, an anti-inflammatory activity (Paulsen et al, 2002), the regulation cell growth and cell differentiation (Liao et al, 2012) and protection in the development of tumors (Kanwar et al, 2013). In RA, LTF functions as a so-called survival factor for neutrophils in the synovial fluid (Wong et al, 2009). MTX reduces the expression of LTF mRNA (Oshida et al, 2011). In the work published by von Koczan et al. (2008), LTF, in addition to another 43 genes, was described as a predictive gene for the therapy with etanercept, an anti-TNF biologic. The investigations, however, did not relate to baseline gene expression prior to therapy alone, but were dependent on a second investigation a few days after the commencement of therapy and thus did not have a predictive value, but rather a prognostic value.

15. The Olfactomedin 4 (OLFM4) mRNA coding for the protein which is assigned to the noelin gene family is strongly expressed during myeloid cell development and was described for the first time in myeloblasts (Zhang et al, 2002). The protein OLFM4 is expressed in the endoplasmic reticulum, has an anti-apoptotic function and, inter alia, also promotes tumor growth (Park et al, 2012). OLFM4 inhibits the cell growth of prostate tumor cells and suppresses bone metastasis via negative interaction with cathepsin D and the chemokine (C-X-C motif) ligand 12 (a.k.a.: SDF-1; Berger 1988). In systemic lupus erythematosus and in inflammatory bowel diseases, OLFM4 has been described as a diagnostic and prognostic marker in connection with other markers (U.S. Pat. No. 8,148,067 B2, U.S. Pat. No. 8,148,067 B2). To date, there is no knowledge regarding the role and expression of OLFM4 in RA. However, in inflammatory bowel diseases, it has been described that OLFM4 comes into question as a diagnostic biomarker and as regards the immune response after bacterial infections, autophagic processes are regulated via cathepsin-D involvement (Montero-Melendez et al, 2013).

16. Matrix Metalloproteinase-8 (MMP8), like the biomarkers for the MTX prediction of the HLA-DRB4-positive RA sub-group described above, is capable of degrading type II collagen (Billinghurst et al, 1997). No connection with MTX has as yet been found. Both in osteoarthritis and also in RA, matrix metalloproteinases play a decisive role in the destruction of ligament structures (Shlopov et al, 1997). On the other hand, the proteins can be detected in the serum and in the synovial fluid in RA patients (Tchetverikov et al, 2004). Neither a direct nor indeed an indirect interaction between MMP8 and MTX has as yet been discovered.

The present invention will now be described in more detail in the following figures and examples without in any way being limited thereto. The references which are cited are hereby fully incorporated herein by reference.

### FIGURES

FIG. 1. Preselection for predictive mRNA biomarkers for MTX monotherapy.

Gene selection using the criteria described above for paired Affymetrix comparisons between responders and non-responders (n=52 RA patients) provided a count of 14 biomarkers in the pre-analysis. Because of its “plus/minus” regulation behavior, the selection marker HLA-DRB4 (ID: 209728_at) described in the following analyses and the claims as the selection marker had a decisive effect on classifying the RA patients into the HLA-DRB4-positive and HLA-DRB4-negative sub-population and is indicated in the hierarchical cluster analysis representation by a rectangle ( [symbol] ). The cluster analysis using Genesis was carried out by log transformation with associated Pearson analysis.

FIG. 2. Hierarchical cluster analysis of the HLA-DRB4-positive and the HLA-DRB4-negative patients sub-groups between responders and non-responders.

Having regard to the classification into HLA-DRB4-positive and HLA-DRB4-negative RA patients sub-populations, in accordance with the described conditions (HLA-DRB4 cut-off value, the given fold change value and the “increased/decreased” reference values) within the paired comparison between responders and non-responders, respectively n=16 biomarkers were defined as biomarker gene sets. The cluster analysis using Genesis was carried out by log transformation with associated Pearson analysis. Within the HLA-DRB4-negative patient sub-group, for a clear separation, the sensitivity and specificity were respectively 100%, while the HLA-DRB4-positive RA-patients sub-group exhibited a sensitivity of 83.3% and a specificity of 92.9%.

FIG. 3. Hierarchical cluster analysis of the HLA-DRB4-positive and the HLA-DRB4-negative patients sub-group between responders and non-responders incorporating the moderate responder group.

Having regard to classification into HLA-DRB4-positive and HLA-DRB4-negative RA patients sub-populations, in accordance with the described conditions (HLA-DRB4 cut-off value, the given fold change value and the “increased/decreased” reference values) within the paired comparison between responders and non-responders, hierarchical cluster analyses incorporating the moderate responders were carried out. The cluster analysis using Genesis was carried out by log transformation with associated Pearson analysis. In this regard, again, for the HLA-DRB4-negative RA-patients sub-group there was a clear separation between the responders and the non-responders with a specificity and sensitivity of 100%. For the HLA-DRB4-positive RA patient sub-group, a sensitivity of 100% and a specificity of 92.9% (without considering the moderate responders) and 95.7% (with the moderate responders which were assessed as responders) were obtained.

FIG. 4. Validation of the Affymetrix gene selection via quantitative real time PCR.

This shows exemplary results of the validations for prediction of the therapy response to MTX, via quantitative real time qPCR with assessments in triplicate: (A) HLADRB4; (B) RNASE2; (C) MMP8.

The y axis represents the gene expression of the individual candidate genes with respect to the housekeeping gene ‘Ribosomal protein Large P0’ (RPLP0). In this regard, the specific biomarker for the HLA-DRB4-negative group, and also the biomarker selection for the HLA-DRB4-positive sub-group were compared. The graph was produced by means of a box plot method using SPSS software. The lines represent the mean values and the bars show the standard deviation within the comparisons between the MTX responders (R), the moderate responders (MR) and the non-responders (NR). The stars indicate absolute deviations which are not in the defined range.

FIG. 5. Validation of the Affymetrix gene selection via quantitative real time PCR.

This shows the results of validations for the prediction of the therapy response to MTX, via quantitative real time qPCR with assessments in triplicate.

(A) ARG1, CKAP4, CRISP3, CST3, GCLM, KIAA0564;

(B) KIAA1324, LCN2, LTF, MMP8, OLFM4, OSBPL1A;

(C) SIAH1, SLC8A1, SULF2, HLA-DRB4.

The y axis represents the gene expression of the individual candidate genes with respect to the housekeeping gene “Ribosomal Protein Large P0” (RPLP0). In this regard, the specific biomarkers for the HLA-DRB4-negative group were compared. The graph was produced by means of a box plot method using SPSS software. The lines represent the median values and the bars show the standard deviation within the comparisons between the MTX responders (R), the moderate responders (MR) and the non-responders (NR). The stars indicate absolute deviations which are not in the defined range.

FIG. 6. Validation of the Affymetrix gene selection via quantitative real time PCR.

This shows the results of validations, for the prediction of the therapy response to MTX, via quantitative real time qPCR with assessments in triplicate.

(A) AQP3, CFD, DEFA4, EIF5A, GATA3, KCNE3;

(B) PAM, PRDX5, RNASE2, SLC35E2, SNHG5, SPAG9;

(C) TCN1, TKT, WLS.

The y axis represents the gene expression of the individual candidate genes with respect to the housekeeping gene “Ribosomal Protein Large P0” (RPLP0). In this regard, the specific biomarkers for the HLA-DRB4-negative group were compared. The graph was produced by means of a box plot method using SPSS software. The lines represent the median values and the bars show the standard deviation within the comparisons between the MTX responders (R), the moderate responders (MR) and the non-responders (NR). The stars indicate absolute deviations which are not in the defined range.

### EXAMPLES

### Example 1

### mRNA Biomarkers

1. Methods

1.1 Patient Samples

52 patients with a clinically confirmed RA were examined in the series of tests for the identification and definition of biomarkers for therapy prediction with MTX. In this regard, 5 ml of whole blood was taken from the patients in 2 PAXgene tubes (PreAnalytiX, Hombrechtikon, Switzerland) and turned for 24 hours on an overhead roller at 20° C. (20 rpm); afterwards, they were frozen at −20° C. until worked up. The patient samples were acquired in the context of two clinical studies under standard conditions (HitHard study; n=29; own clinical study; n=22) and after authorization by the ethics commission of the charity, as well as the agreement of the patients. The clinical data, prior to MTX therapy and over the course of >1 year, were stored in the context of the study conditions in a clinical database in accordance with ISO 9001 standard guidelines. The calculation of the assessment of the MTX response before and during the therapy periods was carried out in accordance with the guidelines from the European League Against Rheumatism (ELUAR) using the “Disease Activity Score” considering 28 joints (DAS28; (van Gestel et al, 1999)). The MTX therapy response was classified, in accordance with the guidelines, into the following three groups: responders (R), moderate responders (MR) and non-responders (NR).

1.2 Preparation of Total RNA

The stored and frozen PAXgene blood tubes were thawed, following the recommendations of the manufacturer, for two hours at ambient temperature and the RNA was prepared with the PAXgene® Blood miRNA Kit (PreAnalytiX). This kit allows both mRNA and also miRNA transcription analyses to be carried out. The quantity of total purified RNA was recorded in a NanoDrop 1000® UV-Vis spectrophotometer (Thermo Fisher Scientific Inc., NanoDrop, Wilmington, Del., USA) and the quality test was carried out using the 2100® Bioanalyzer (Agilent Technologies Inc., Santa Clara, Calif., USA).

1.3 Microarray Analyses

Prior to using the samples for microarray analysis, globin mRNA was reduced using the GLOBINclear™ Kit (Life Technologies, Ambion, USA), following the instructions of the manufacturer. Next, the complementary DNA (cDNA) was synthesized along with in vitro transcription into cRNA using the Affymetrix GeneChip® 3'IVT Express Kit (Affymetrix, Santa Clara, Calif., USA). The amplified and biotin-labelled cRNA was then hybridized on the GeneChip® Human Genome U133 Plus 2.0 array for 16 hours at 45° C., following the instructions of the manufacturer. The washing and labelling steps were carried out in a GeneChip® Fluidics Station 450GeneChip® using the hybridization, washing and labelling kits from Affymetrix. The hybridization signal was read in an Affymetrix GeneChip® 3000 7G Scanner, with subsequent normalization using the Affymetrix MAS5.0 algorithm from Expression Console Software.

1.4 Statistical Analysis and Hierarchical Cluster Method for the Microarray Results

The differential mRNA gene expression was evaluated using the BioRetis online database (BioRetis GmbH, Berlin). In this regard, the data were pre-filtered applying the criteria of ≧70% in all group comparisons (for example R versus NR) and a fold change of ≧1.5 or ≦−1.5. The limiting value for the signal strength within the paired group comparisons (responder versus non-responder) without and with the moderate responders was set to at least ≧50 in one of the two comparison groups. The data were viewed via Genesis 1.7.6 hierarchical cluster software (Gene Expression Similarity Investigation Suite; Graz, University, Austria; (Sturn et al, 2002) using log transformation and Pearson analysis. Correlation analyses of the mRNA sample set (gene-) signals, the clinical data, and vice versa were determined using the 1- and 2-tailed Wilcoxon rank test with the aid of the IBM SPSS Statistics v.22 software (Stacon, Witzenhausen, Germany).

1.5 Validation of the Microarray Analyses Via Quantitative qPCR

The examination of the Affymetrix-based results the biomarkers defining differential gene expression was carried out with an independent method using quantitative Real Time PCR (qPCR). In this regard, standardized RT² Primer Assays (Qiagen; Hilden, Germany) and, for detection, Power SYBR® Green PCR Master Mix (Lifetechnologies, Applied Biosystems, USA), were employed. The assessment was carried out by normalizing the gene expression of the individual candidate genes with respect to the “Ribosomal Protein Large P0” (RPLP0) housekeeping gene. The qPCR runs were carried out in a StepOne Plus® Real Time Cycler (Lifetechnologies, Carlsbad, Calif., USA). The amplification of the amplification efficiencies and calculation of the efficiency-corrected delta-delta-Ct (ΔΔCt) values were assayed with the aid of the MS Excel 2010 software program (Microsoft, Redmont, Wash., USA) and the graphs were visualized by means of the SPSS software program.

2. Results

The identification and definition of biomarkers in whole blood to predict the outcome of treatment with MTX before therapy even commenced was carried out with 52 patient samples from the two independent studies (internal study (n=23) RA; HitHard Study (n=29); Detert et al, 2013). Both studies were compatible as regards the inclusion criteria. The mean duration of disease in the RA patients from the internal studies was 15.6 months (SD=48.9; SEM-22.3) and for patients from the HitHard Study, on average 1.7 months (SD=1.9; SEM=1.4). Apart from the difference in the duration of disease, there were statistically no anomalies within the other parameters raised. The calculation of the disease activity and the future response rate to drug with MTX was carried out in accordance with the definition by the European League Against Rheumatism (EULAR) in accordance with the DAS28 classification criteria (van Gestel et al, 1996).

See also Table 1 for clinical and laboratory diagnostic data for the 52 RA patients before and during treatment with MTX.

No or only very low correlations appeared within the genders, the so-called Visual Analog Squares (VAS), the questionnaire (Health Assessment Questionnaire, (HAQ)), the subjective assessment by the patients themselves and objective assessment by the attending physician, the titer of the C-reactive Proteins (CRP), the blood sedimentation rate (BSR), the number of swollen joints (based on 28) and the number of painful joints (based on 28).

The first approach to classification into responders and non-responders, with and without incorporating the MR resulted, in the 52 patient samples, in no desirable success with a clear separation. In this regard, the following criteria were set via the database query in BioRetis (online database from BioRetis GmbH, Berlin): minimal change call with an agreement of ≧30% increase/decrease within the group comparisons (R vs. NR) and a fold change (FC) of ≧|1.5| (| |=sum). The analysis resulted in a candidate gene set of 14 genes. The HLA-DRB4 mRNA was a biomarker gene from this pre-selection.

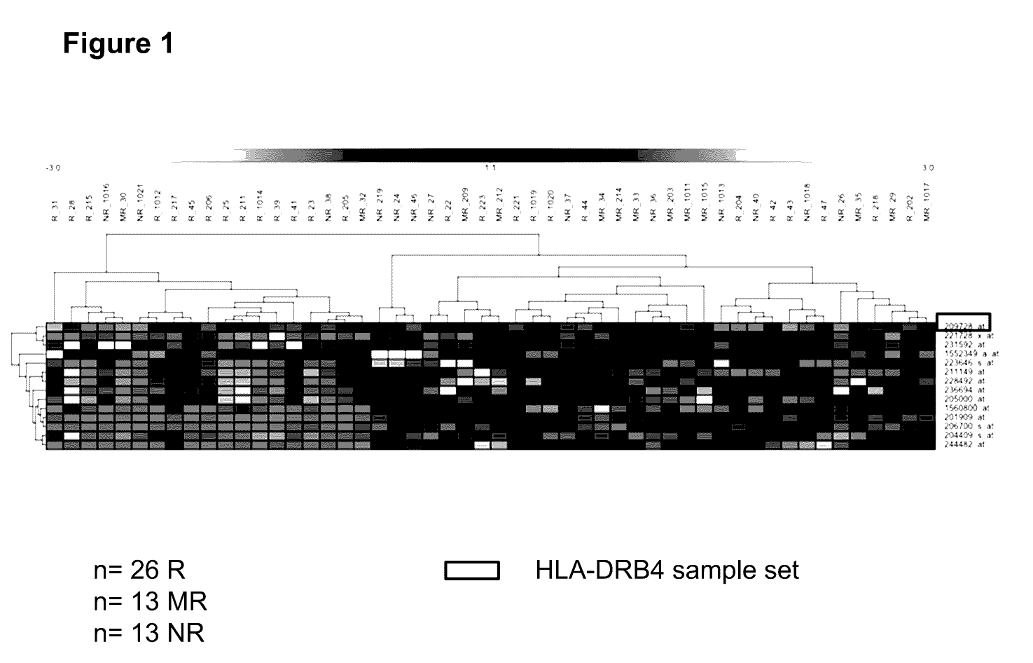

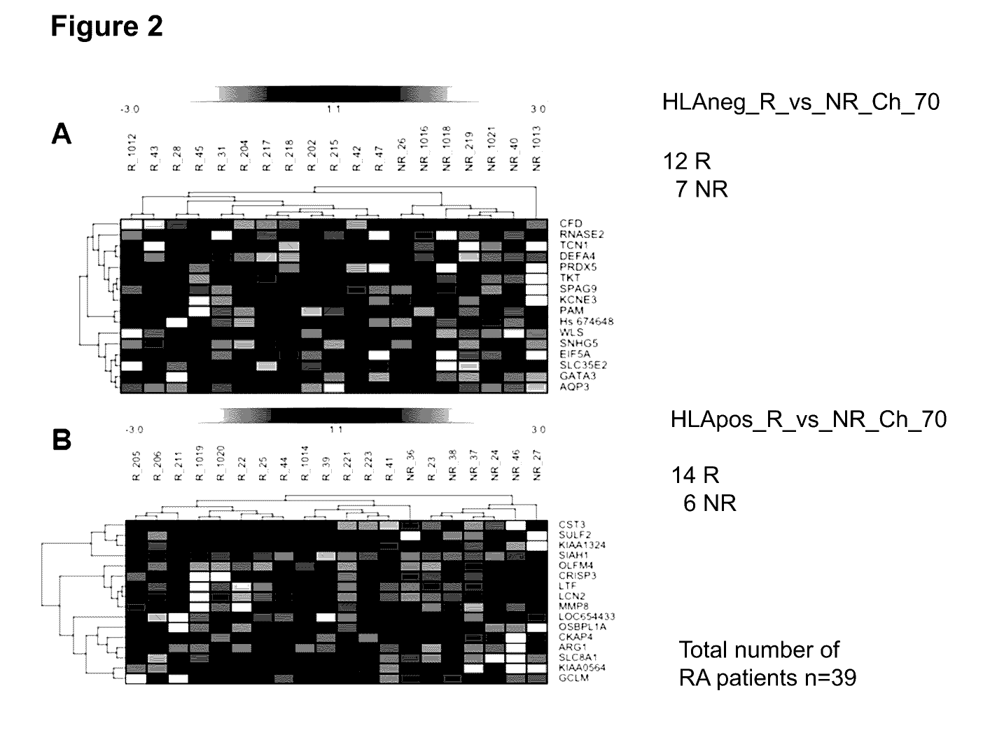

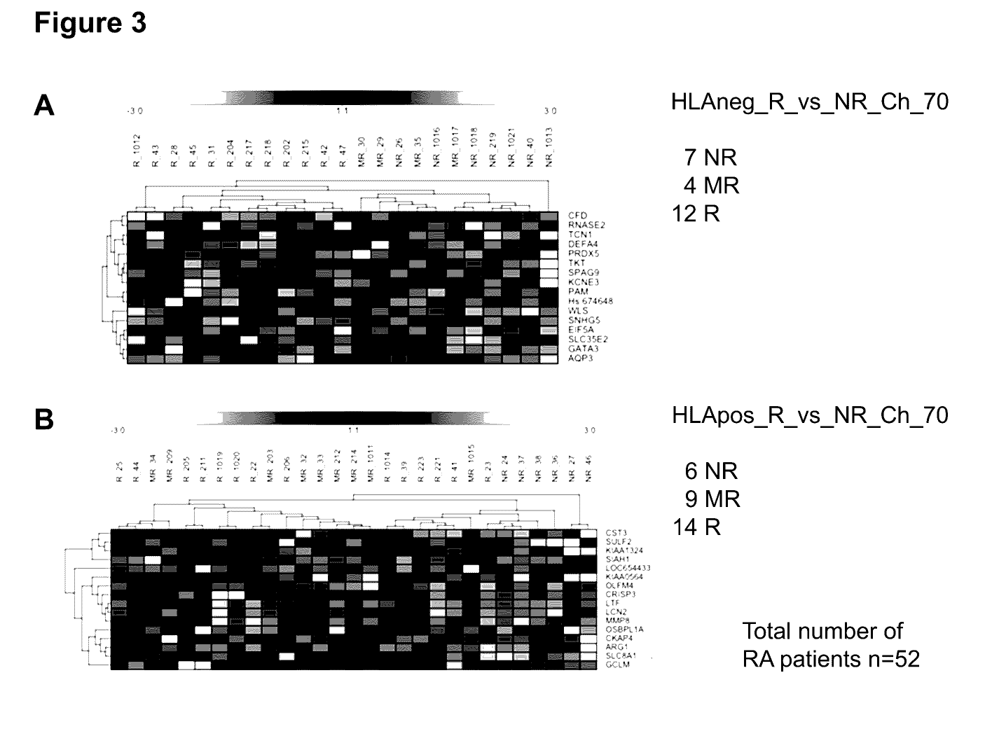

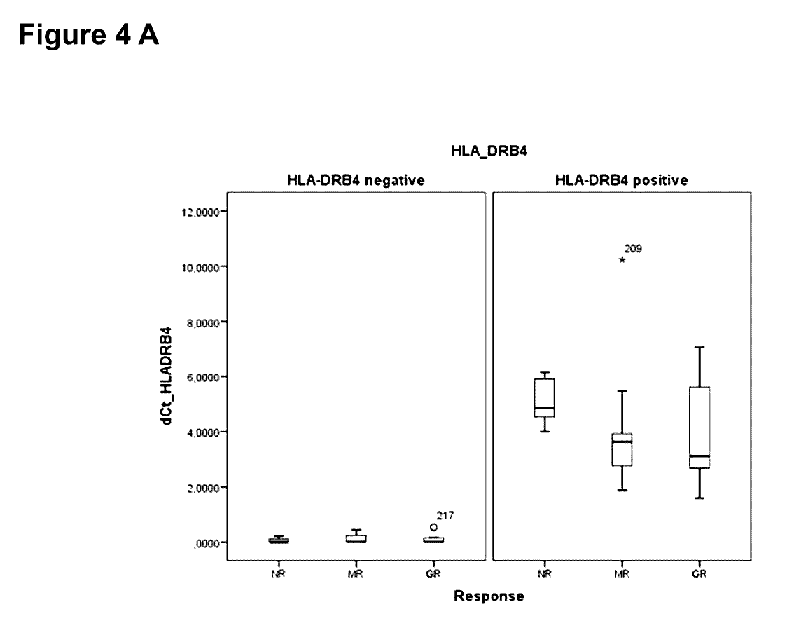

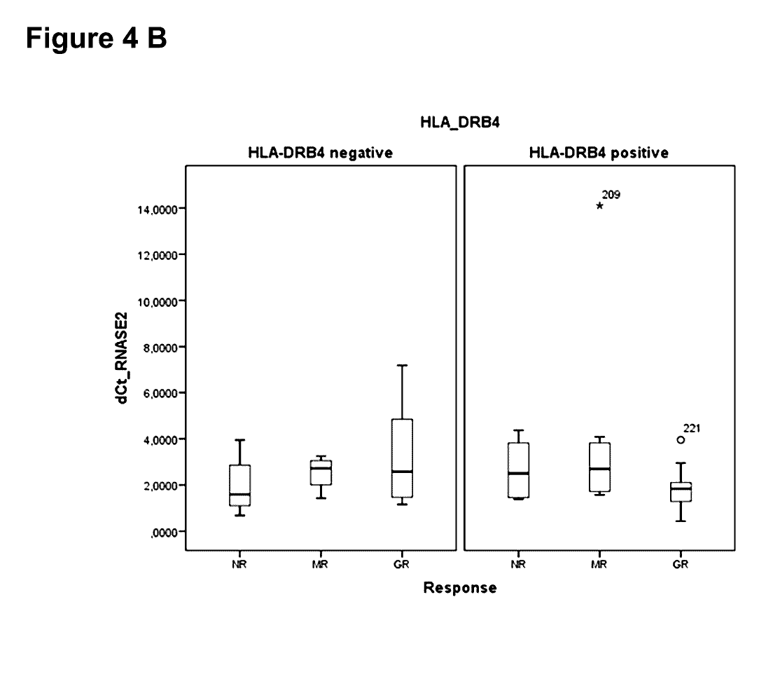

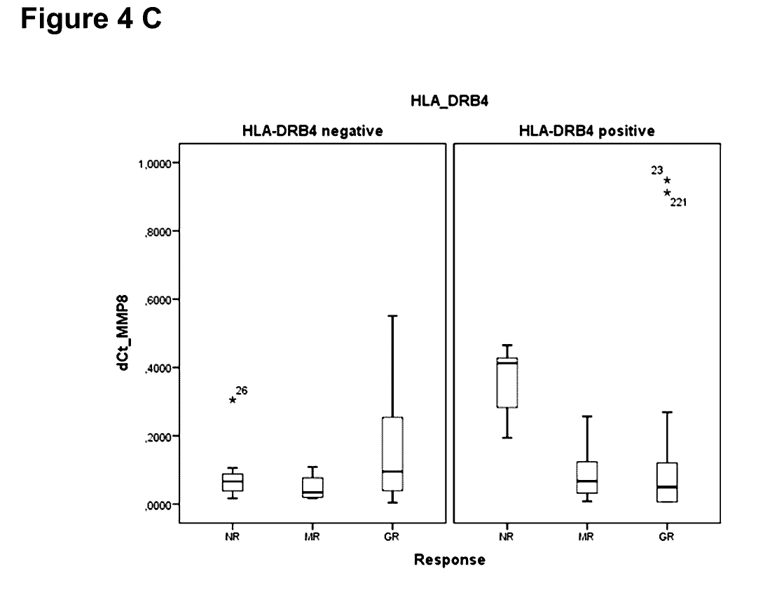

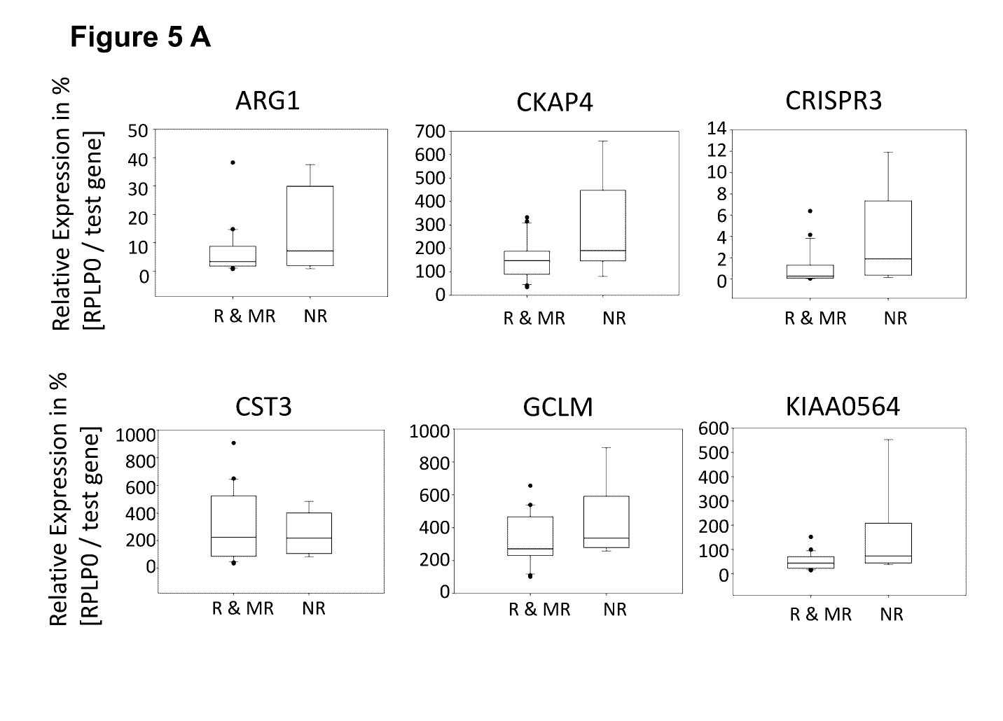

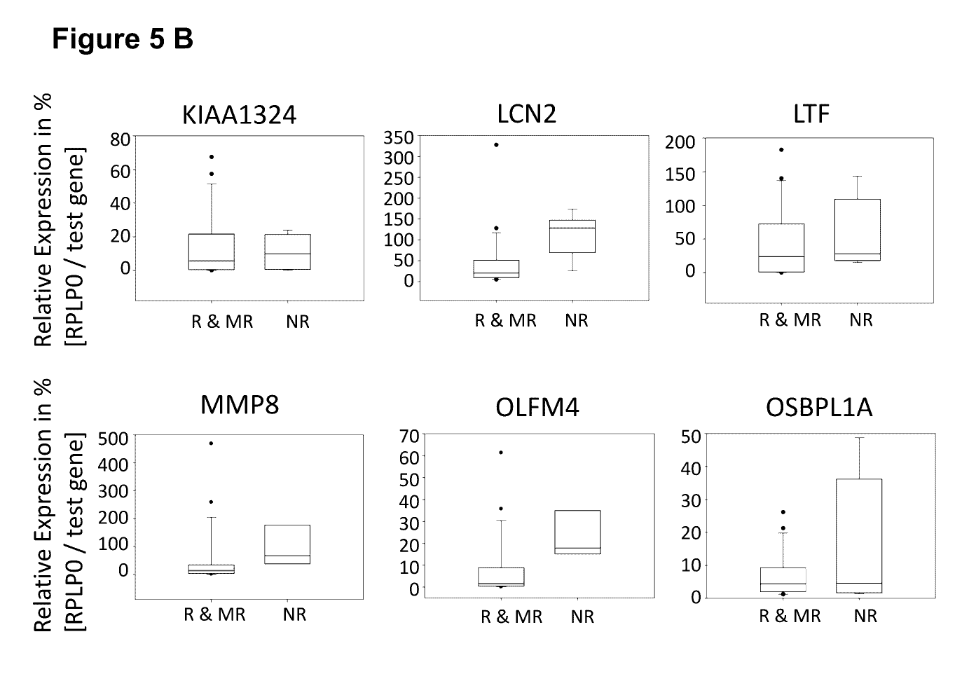

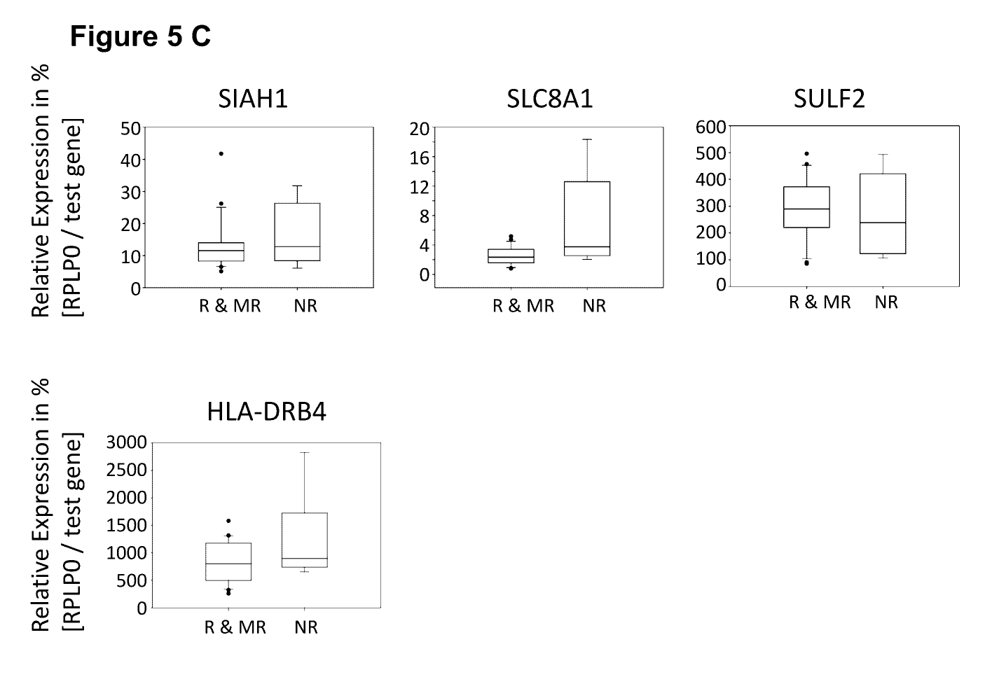

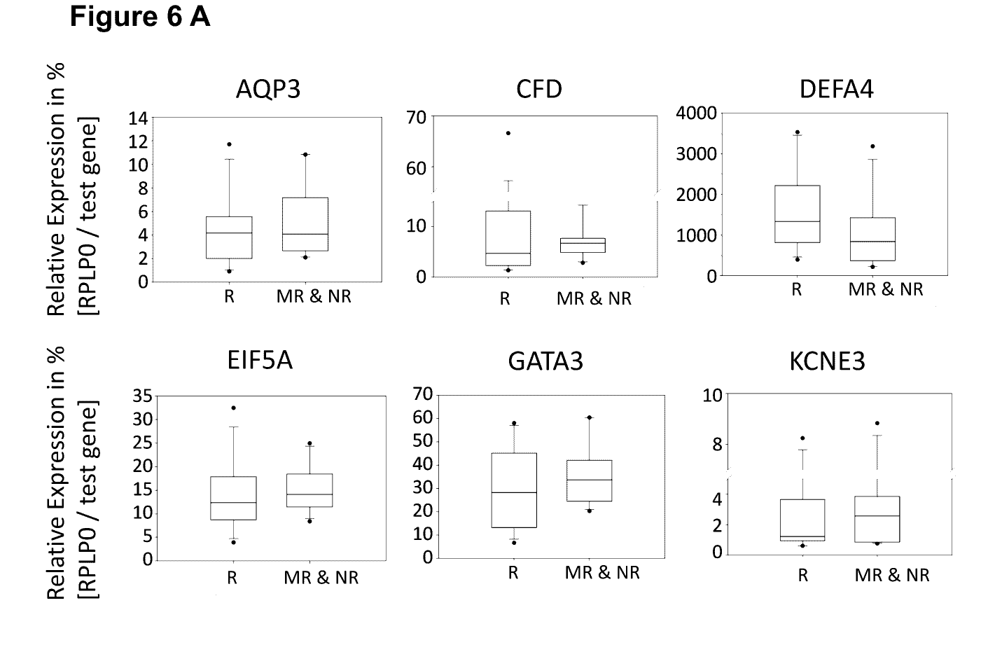

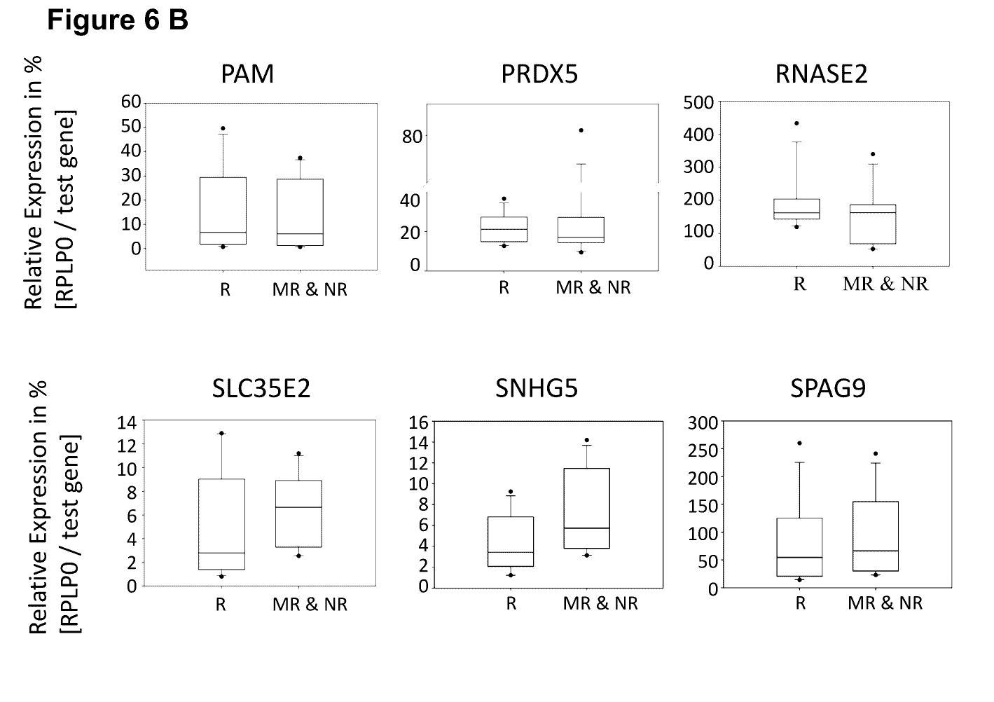

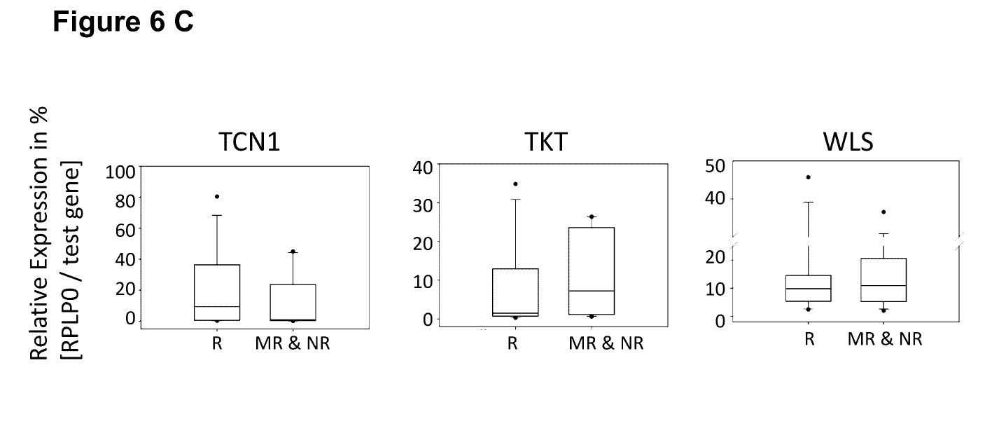

Even with incorporation of the moderate responders, no clear separation could be ascertained between the responder and non-responder group (FIG. 1).

In order to carry out an investigation using transcription analysis employing Affymetrix microarrays, the patient samples came from our own clinical study (total n=29; responder n=14; non-responder n=6) and from the HitHard study (total n=23; responder n=12; non-responder n=7). By incorporating the pre-selection markers HLA-DRB4 (Affymetrix ID: 209728_at), it was possible, using the gene sets defined via BioRetis (BioRetis database, BioRetis GmbH, Berlin) (16 genes for each of the HLA-sub-groups) to obtain precise separations with a sensitivity of 100% and specificity of 96% in the HLA-DRB4-positive patient group between the responders and the non-responders. The selection criteria for the interrogation for identifying the two HLA-DRB4 sub-group-specific genes between the responder and the non-responder groups were: agreements of at least 70% (increase/decrease; see Tables 2 and 3) within the paired individual comparisons (R vs. NR) and a mean regulation factor (fold change; FC) of ≧|1.5|.

The separation within the HLA-DRB4-negative sub-population reached a respective sensitivity and specificity of 100%. It was presented as a hierarchical cluster analysis using the Genesis software tool (FIGS. 2A and 2B).

When moderate responders (MR; n=9) were added, they clustered within the responder group in the HLA-DRB4-positive sub-group, with just a single MTX non-responder being falsely categorized. The sensitivity was 100% and the specificity was 93%. In the HLA-negative sub-population of the RA patients, the moderate responders (n=4) clustered within the non-responder group and all MTX responders separately in a clearly distinct group. In this regard, the sensitivity as well as the specificity were each 100% (FIGS. 3A and 3B).

HLA-DRB4 (Affymetrix ID: 209728_at), which contributed to clear separation as an additional selection marker within the system, had already been described as a product marker with diagnostic relevance in RA (Heldt et al. 2003). Within the HLA-DRB4-positive (n=29) and negative sub-group (n=23), there was a clear and also a significant difference in gene expression by visible signal intensity differences in the respective patients.

Nevertheless, it was clear that the quality of the differentiation between responders and non-responders was not sufficient to come even close to satisfying the quality criteria for a predictive diagnostic test of the required quality. This is also highlighted by the fact that the minimum number for the two sub-group-specific biomarkers was a requirement, and leaving out any individual resulted in a reduced quality in the categorization of the MTX therapy predictions.

The gene sets for the HLA-DRB4-negative sub-group and the HLA-DRB4 were validated using quantitative RT-qPCR and produced a relatively clear consistency of regulation within the respective groups (responder and non-responder). See FIG. 4 with exemplary results for validation.

### Example 2

### Further Validation of mRNA Biomarkers

2.1 Preparation of Total RNA (Total RNA)

Prior to the MTX treatment, whole blood samples were collected in PAXgene® blood tubes (PreAnalytiX, Hombrechtikon, Switzerland), incubated for 24 h by rolling and then stored at −20°. The stored and frozen PAXgene® blood tubes were thawed, following the instructions of the manufacturer, for two hours at ambient temperature and the RNA was prepared with the PAXgene® Blood miRNA Kit (PreAnalytiX). This kit allowed both mRNA and also miRNA transcription analyses to be carried out. The quantity of the purified total RNA was determined in the NanoDrop 1000® UV-Vis spectrophotometer (Thermo Fisher Scientific Inc., NanoDrop, Wilmington, Del., USA) and the quality control was carried out using the 2100® Bioanalyzer (Agilent Technologies Inc., Santa Clara, Calif., USA).

2.2 Validation Using Quantitative RT-qPCR

The examination of the Affymetrix-based results for differential gene expression of 30 of the 32 defined biomarkers was carried out using an independent method via quantitative Real Time PCR (qPCR). In this regard, standardized RT² primer assays (Qiagen; Hilden, Germany) and, for detection, Power SYBR® Green PCR Master Mix (Lifetechnologies, Applied Biosystems, USA), were used. In the case of two of the defined biomarkers, at the time of the experiment, no commercial RT² primer assays were available. The assessment was carried out by normalizing the gene expression of the individual candidate genes with respect to the housekeeping gene used, “Ribosomal Protein Large P0” (RPLP0). The qPCR runs were carried out in a StepOne Plus® Real Time Cycler (Lifetechnologies, Carlsbad, Calif., USA). Amplification efficiencies and efficiency-corrected delta-delta-Ct (ΔΔCt) values were calculated as described in Fleige et al, 2006.

The statistical evaluation of the differential gene expression between responders, moderate responders and non-responders was carried out with REST 2009 software (Qiagen, Pfaffl et al, 2002). The individual delta-Ct values were presented using SPSS (Systat). The means of the microarray-FC and delta-delta-CT values from the RT-qPCR were compared using t-Test-statistics. The non-parametric. Wilcoxon rank and Kruskal-Wallis tests were for the future response to MTX treatment and the corresponding clinical values before and after therapy. Correlations between Bonferroni-corrected results from microarray and RT-qPCR tests were investigated using the Pearson- and Spearman rank tests with SPSS (Systat).

Results:

The gene sets for the HLA-DRB4-negative sub-group and the HLA-DRB4-positive sub-group were validated using quantitative RT-qPCR and revealed a relatively clear consistency of regulation within the respective groups (responders and non-responders).

See FIGS. 5 and 6 for more results for the validation. See also Table 5 for the RT-qPCR results and their correlation with the microarray data.

The following markers of the HLA-DRB4+ group:
CKAP4, CRISP3, KIAA0564, LCN2, MMP8, OLFM4, SLC8A1 and the following markers of the HLA-DRB4-group:
AQP3, DEFA4, SNHG5 produced a mean regulation factor |FC| of ≧1.5=signal; p value qPCR <0.1; correlation with the microarray data at least >0.5.

The following markers of the HLA-DRB4+ group:
CRISP3, LCN2, MMP8, OLFM4 produced a mean regulation factor |FC| of >3=signal; p value qPCR <0.1; correlation with the microarray data at least >0.5.

For details, see Table 5.

### Example 3

### miRNA Biomarkers

Methods:

In addition to the biomarkers named in the description for the prediction of the therapy with MTX, miRNA expression profiles of n=39 patients from the two clinical studies defined above were assayed.

The purified total RNA was processed using the Affymetrix Flash-Taq™ biotin HSR RNA Labeling Kit (Genisphere, Hatfield, Pa., USA). Hybridization of the labelled samples was carried out for 16 hours at 45° C. with miRNA 2.0 microarrays, following the instructions from the manufacturer, in the GeneChip® Fluidics Station 450. The hybridization signals were selected in the Affymetrix GeneChip® 3000 7G Scanner and normalization of the data was carried out after washing the samples with the miRNA QCTool Software Version 1.1.1.0 (Affymetrix).

Results:

In total, n=7 miRNA biomarkers could be identified. By adding in the moderate responders (n=13), the sensitivity, with two exceptions, between the responder group (n=18) and the non-responder group (n=8) was 100% and the specificity was 94.9%.

TABLE 5
Predictive miRNA biomarkers

| | (mature) | Accession No. | SEQ ID |
|---|---|---|---|
| miRNA | nucleotide sequence | miRBase** | NO. |
| Hsa-mir-572_st | GUCCGCUCGGCGGUGGCCCA | MI0003579 | 34 |
| | | MIMAT0003237 | |
| Hsa-mir-1915_st | ACCUUGCCUUGCUGCCCGGGCC | MI0008336 | 35 |
| | | MIMAT0007891 | |
| Hsa-mir-223_st | CGUGUAUUUGACAAGCUGAGUU | MI0000300 | 36 |
| | | MIMAT0004570 | |
| Hsa-mir-193b_st | CGGGGUUUUGAGGGCGAGAUGA | MI0003137 | 37 |
| | | MIMAT0004767 | |
| Hsa-mir-3177_st | UGUGUACACACGUGCCAGGCGCU | MI0014211 | 38 |
| | | MIMAT0019215 | |
| Hsa-mir-4298_st | CUGGGACAGGAGGAGGAGGCAG | MI0015830 | 39 |
| | | MIMAT0016852 | |
| Hsa-mir-1184_st | CCUGCAGCGACUUGAUGGCUUCC | MI0005829 | 40 |
| | | MIMAT0005829 | |

**Accession No. of the stem-loop sequence (italics) and Accession No. of the mature miRNA-nucleotide sequence (Source: miRBase database, www.mirbase.org)

Statistics:

Correlation analyses between the mRNA and miRNA signals, the clinical parameters and the candidate genes were carried out using the 1- and 2-tailed Wilcoxon rank test.

### REFERENCES

Abolmaali S S et al. (2013). A review of therapeutic challenges and achievements of methotrexate delivery systems for treatment of cancer and rheumatoid arthritis. Cancer Chemother Pharmacol. 71(5):1115-30.

Anderson J J et al. (2000) Factors predicting response to treatment in rheumatoid arthritis: the importance of disease duration. arthritis and rheumatism 43, 22-29.

Arpegard J et al. (2008) Cystatin C—a marker of peripheral atherosclerotic disease? Atherosclerosis 199, 397-401.

Barra L et al. (2014) Efficacy of biologic agents in improving the Health Assessment Questionnaire (HAQ) score in established and early rheumatoid arthritis: a meta-analysis with indirect comparisons. Clin Exp Rheumatol. 2014 Jan. 24. [Epub ahead of print]

Belinsky G S et al. (2007) The contribution of methotrexate exposure and host factors on transcriptional variance in human liver. Toxicological sciences : an official journal of the Society of Toxicology 97, 582-594.

Berger H (1988) “The paradigm shift' and patient preference. Hospital practice 23, 16

Billinghurst R C et al. (1997) Enhanced cleavage of type II collagen by collagenases in osteoarthritic articular cartilage. The Journal of clinical investigation 99, 1534-1545

Blaser J et al. (1995) A sandwich enzyme immunoassay for the determination of neutrophil lipocalin in body fluids. Clinica chimica acta; international journal of clinical chemistry 235, 137-145.

Blom M et al. (2009) The reason for discontinuation of the first tumor necrosis factor (TNF) blocking agent does not influence the effect of a second TNF blocking agent in patients with rheumatoid arthritis. The Journal of rheumatology 36, 2171-2177.

Chaudhry M A (2013) expression Pattern of Small Nucleolar RNA Host Genes and Long non-Coding RNA in X-rays-Treated Lymphoblastoid Cells. International journal of molecular sciences 14, 9099-9110.

Cheok M H et al. (2003) Treatment-specific changes in gene expression discriminate in vivo drug response in human leukemia cells. Nature genetics 34, 85-90.

Cohen S B et al. (2008) Unresolved issues in identifying and overcoming inadequate response in rheumatoid arthritis: weighing the evidence. The Journal of rheumatology. Supplement 81, 4-30; quiz 31-34.

Criswell L A et al. (2004) The influence of genetic variation in the HLA-DRB1 and LTA-TNF regions on the response to treatment of early rheumatoid arthritis with methotrexate or etanercept. arthritis and rheumatism 50, 2750-2756.

Dai Y et al. (2005) HSulf-1 and HSulf-2 are potent inhibitors of myeloma tumor growth in vivo. The Journal of biological chemistry 280, 40066-40073

the Broeder A A et al. (2002) Long term anti-tumor necrosis factor alpha monotherapy in rheumatoid arthritis: effect on radiological course and prognostic value of markers of cartilage turnover and endothelial activation. Annals of the rheumatic diseases 61, 311-318.

Detert J et al (2013) Induction therapy with adalimumab plus methotrexate for 24 weeks followed by methotrexate monotherapy up to week 48 versus methotrexate therapy alone for DMARD-naive patients with early rheumatoid arthritis: HIT HARD, an investigator-initiated study. Annals of the rheumatic diseases 72, 844-850.

Dong X Y et al. (2008) SnoRNA U50 is a candidate tumor-suppressor gene at 6q14.3 with a mutation associated with clinically significant prostate cancer. Human molecular genetics 17, 1031-1042.

Dong X Y et al. (2009) Implication of snoRNA U50 in human breast cancer. Journal of genetics and genomics=Yi chuan xue bao 36, 447-454.

Fleige, S., Walf, V., Huch, S., Prgomet, C., Sehm, J. & Pfaffl, M. W. Comparison of relative mRNA quantification models and the impact of RNA integrity in quantitative real time RT-PCR. Biotechnology letters 28, 1601-13 (2006).

Foller M et al. (2013) Functional significance of glutamate-cysteine ligase modifier for erythrocyte survival in vitro and in vivo. Cell death and differentiation 20, 1350-1358

Franck, T et al. (2006) Mutation analysis of the seven in absentia homolog 1 (SIAH1) gene in Parkinson's disease. Journal of neural transmission 113, 1903-1908.

Furst D E (1996) Clinical pharmacology of combination DMARD therapy in rheumatoid arthritis. The Journal of rheumatology. Supplement 44, 86-90.

Ganz T et al (1990) Defensins. European journal of haematology 44, 1-8.

Gulbahce H E et al. (2013) Significance of GATA-3 expression in outcomes of patients with breast cancer who received systemic chemotherapy and/or hormonal therapy and clinicopathologic features of GATA-3-positive tumors. Human pathology 44, 2427-2431.

Haberhauer G and Peichl P (1989) [The effect of HLA-DRB4 on the clinical picture of chronic polyarthritis]. Zeitschrift fur Rheumatologie 48, 129-131.

Han W et al. (2006) Genomic alterations identified by array comparative genomic hybridization as prognostic markers in tamoxifen-treated estrogen receptor-positive breast cancer. BMC cancer 6, 92.

Hansen T et al. (2000) Synovial giant cells in rheumatoid arthritis: expression of cystatin C, but not of cathepsin B. Experimental and toxicologic pathology: official journal of the Gesellschaft fur Toxikologische Pathologic 52, 312-316.

Hayashi T et al. (2010) Elevated level of serum cystatin-C concentration is a useful predictor for myelosuppression induced by methotrexate for treatment of rheumatoid arthritis. Modern rheumatology/the Japan Rheumatism Association 20, 548-555.

Heldt C et al (2003) Differential expression of HLA class II genes associated with disease susceptibility and progression in rheumatoid arthritis. arthritis and rheumatism 48, 2779-2787.

Hu, G., Chung, Y. L., Glover, T., Valentine, V., Look, A. T., and Fearon, E. R. (1997) Characterization of human homologs of the Drosophila seven in absentia (sina) gene. Genomics 46, 103-111.

Huang L W et al. (2001) Arginase levels are increased in patients with rheumatoid arthritis. The Kaohsiung journal of medical sciences 17, 358-363.

Ishibashi K et al. (1995) Structure and chromosomal localization of a human water channel (AQP3) gene. Genomics 27, 352-354.

Ishida N and Kawakita M (2004) Molecular physiology and pathology of the nucleotide sugar transporter family (SLC35). Pflugers Archiv : European journal of physiology 447, 768-775. Ivanenkov Y A and Chufarova N V (2014) Small-molecule arginase inhibitors. Pharmaceutical patent analyst 3, 65-85.

Johansson M et al. (2005) The oxysterol-binding protein homologue ORP1L interacts with Rab7 and alters functional properties of late endocytic compartments. Molecular biology of the cell 16, 5480-5492.

Jouvin M H and Kazatchkine M (1983) [The alternative complement pathway]. Pathologie-biologie 31, 839-846.

Kanwar R K and Kanwar J R (2013) Immunomodulatory lactoferrin in the regulation of apoptosis modulatory proteins in cancer. protein and peptide letters 20, 450-458.

Kar N C et al. (1976) Acid, neutral, and alkaline hydrolases in arthritic synovium. American journal of clinical pathology 65, 220-228.

Khananshvili, D. (2013) The SLC8 gene family of sodium-calcium exchangers (NCX)—structure, function, and regulation in health and disease. Molecular aspects of medicine 34, 220-235.

Kjeldsen L et al. (2000) Human neutrophil gelatinase-associated lipocalin and homologous proteins in rat and mouse. Biochimica et biophysica acta 1482, 272-283

Koczan D et al. (2008) Molecular discrimination of responders and nonresponders to anti-TNF alpha therapy in rheumatoid arthritis by etanercept. arthritis research & therapy 10, R50.

Laine M et al. (2007) Low salivary dehydroepiandrosterone and androgen-regulated cysteine-rich secretory protein 3 levels in Sjogren's syndrome. arthritis and rheumatism 56, 2575-2584.

Landro L et al. (2008) Decreased serum lipocalin-2 levels in human immunodeficiency virus-infected patients: increase during highly active anti-retroviral therapy. Clinical and experimental immunology 152, 57-63.

Lee M H et al. (2008) genes expression profiles of murine fatty liver induced by the administration of methotrexate. Toxicology 249, 75-84.

Li M H et al. (2013) expression of cytoskeleton-associated protein 4 is related to lymphatic metastasis and indicates prognosis of intrahepatic cholangiocarcinoma patients after surgery resection. Cancer letters 337, 248-253.

Li T et al. (2013) Development and Use a Novel combined in-vivo and in-vitro Assay for Anti-inflammatory and Immunosuppressive Agents. Iranian journal of pharmaceutical research : IJPR 12, 445-455.

Liao Y et al. (2012) Biochemical and molecular impacts of lactoferrin on small intestinal growth and development during early life. Biochemistry and cell biology=Biochimie et biologie cellulaire 90, 476-484.

Linnebank M et al. (2005) MTX-induced white matter changes are associated with polymorphisms of methionine metabolism. Neurology 64, 912-913.

Matsuda A et al. (2003) Large-scale identification and characterization of human genes that activate NF-kappaB and MAPK signaling pathways. Oncogene 22, 3307-3318.

Meyer J M et al. (1999) HLA-DRB1 genotype influences risk for and severity of rheumatoid arthritis. The Journal of rheumatology 26, 1024-1034.

Montero-Melendez T et al. (2013) Identification of novel predictor classifiers for inflammatory bowel disease by

Murphy C and Newsholme P (1998) Importance of glutamine metabolism in murine macrophages and human monocytes to L-arginine biosynthesis and rates of nitrite or urea production. Clinical science 95, 397-407

Nakamura Y et al. (2008) The GAS5 (growth arrest-specific transcript 5) gene fuses to BCL6 as a result of t(1;3)(q25;q27) in a patient with B-cell lymphoma. Cancer genetics and cytogenetics 182, 144-149.

Nakayama K and Ronai Z (2004) Siah: new players in the cellular response to hypoxia. Cell cycle 3, 1345-1347.

Nawroth R et al. (2007) Extracellular sulfatases, elements of the Wnt signaling pathway, positively regulate growth and tumorigenicity of human pancreatic cancer cells. PloS one 2, e392

Nishimura M et al. (2009) Tissue-specific mRNA expression profiles of human solute carrier 35 transporters. Drug metabolism and pharmacokinetics 24, 91-99.

O'Dell J R et al. (1998) HLA-DRB1 typing in rheumatoid arthritis: predicting response to specific treatments. Annals of the rheumatic diseases 57, 209-213.

Oshida K et al. (2011) Novel gene markers of immunosuppressive chemicals in mouse lymph node assay. Toxicology letters 205, 79-85.

Otsuki S et al. (2008) expression of novel extracellular sulfatases Sulf-1 and Sulf-2 in normal and osteoarthritic articular cartilage. arthritis research & therapy 10, R61.

Park K S et al. (2012) Olfactomedin 4 suppresses tumor growth and metastasis of mouse melanoma cells through downregulation of integrin and MMP genes. Molecules and cells 34, 555-561

Paulsen F et al. (2002) Antimicrobial peptides are expressed and produced in healthy and inflamed human synovial membranes. The Journal of pathology 198, 369-377.

Paulsson J et al. (2007) Activation of peripheral and in vivo transmigrated neutrophils in patients with stable coronary artery disease. Atherosclerosis 192, 328-334.

Pfaffl, M. W., Horgan, G. W. & Dempfle, L. Relative expression software tool (REST) for group-wise comparison and statistical analysis of relative expression results in real time PCR. Nucleic acids research 30, e36 (2002).

Pfisterer P et al. (1996) CRISP-3, a protein with homology to plant defense proteins, is expressed in mouse B cells under the control of Oct2. Molecular and cellular biology 16, 6160-6168.

Prigge S T et al. (2000) New insights into copper monooxygenases and peptide amidation: structure, mechanism and function. Cellular and molecular life sciences : CMLS 57, 1236-1259.

Quinn M A et al. (2005) Very early treatment with infliximab in addition to methotrexate in early, poor-prognosis rheumatoid arthritis reduces magnetic resonance imaging evidence of synovitis and damage, with sustained benefit after infliximab withdrawal: results from a twelve-month randomized, double-blind, placebo-controlled trial. arthritis and rheumatism 52, 27-35.

Razzaq T et al. (2003) Functional regulation of tissue plasminogen activator on the surface of vascular smooth muscle cells by the type-II transmembrane protein p63 (CKAP4). The Journal of biological chemistry 278, 42679-42685.

Reininger A J et al. (2006) Mechanism of platelet adhesion to von Willebrand factor and microparticle formation under high shear stress. Blood 107, 3537-3545

Rosenberg H F (2008) Eosinophil-derived neurotoxin/RNase 2: connecting the past, the present and the future. Current pharmaceutical biotechnology 9, 135-140.

Rubbert-Roth A and Finckh A (2009) Treatment options in patients with rheumatoid arthritis failing initial TNF inhibitor therapy: a critical review. arthritis research & therapy 11 Suppl 1, S1.

Scally S W et al. (2013) A molecular basis for the association of the HLA-DRB1 locus, citrullination, and rheumatoid arthritis. The Journal of experimental medicine 210, 2569-2582.

Scuoppo C et al. (2012) A tumor suppressor network relying on the polyamine-hypusine axis. Nature 487, 244-248.

Serafini N et al. (2014) Gata3 drives development of RORgammat+ group 3 innate lymphoid cells. The Journal of experimental medicine.

Shen J et al. (2013) Use of folate-conjugated imaging agents to target alternatively activated macrophages in a murine model of asthma. Molecular pharmaceutics 10, 1918-1927

Shlopov B V et al. (1997) Osteoarthritic lesions: involvement of three different collagenases. arthritis and rheumatism 40, 2065-2074

Smolen J S et al. (2010) EULAR recommendations for the management of rheumatoid arthritis with synthetic and biological disease-modifying antirheumatic drugs. Annals of the rheumatic diseases 69, 964-975.

Spitznagel J K (1990) Antibiotic proteins of human neutrophils. The Journal of clinical investigation 86, 1381-1386.

Sturn A et al. (2002) Genesis: cluster analysis of microarray data. Bioinformatics 18, 207-208.

Tanaka R et al. (2000) Intronic U50 small-nucleolar-RNA (snoRNA) host gene of no protein-coding potential is mapped at the chromosome breakpoint t(3;6)(q27;q15) of human B-cell lymphoma. Genes to cells : devoted to molecular & cellular mechanisms 5, 277-287.

Tapinos N I et al. (2002) Characterization of the cysteine-rich secretory protein 3 gene as an early-transcribed gene with a putative role in the pathophysiology of Sjogren's syndrome. arthritis and rheumatism 46, 215-222.

Tchetverikov I et al. (2004) MMP profile in paired serum and synovial fluid samples of patients with rheumatoid arthritis. Annals of the rheumatic diseases 63, 881-883

Tominaga N et al. (2012) Clinicopathological analysis of GATA3-positive breast cancers with special reference to response to neoadjuvant chemotherapy. Annals of oncology : official journal of the European Society for Medical Oncology/ESMO 23, 3051-3057.

Tunca B et al. (2013) Overexpression of CK20, MAP3K8 and EIF5A correlates with poor prognosis in early-onset colorectal cancer patients. Journal of cancer research and clinical oncology 139, 691-702.

Turk V et al. (2008) Cystatins: biochemical and structural properties, and medical relevance. Frontiers in bioscience : a journal and virtual library 13, 5406-5420.

Umeda N et al. (2013) Anti-citrullinated glucose-6-phosphate isomerase peptide antibodies in patients with rheumatoid arthritis are associated with HLA-DRB1 shared epitope alleles and disease activity. Clinical and experimental immunology 172, 44-53.

Umetani M et al. (2001) Function of GATA transcription factors in induction of endothelial vascular cell adhesion molecule-1 by tumor necrosis factor-alpha. Arteriosclerosis, thrombosis, and vascular biology 21, 917-922.

van Gaalen F A et al. (2004) Association between HLA class II genes and autoantibodies to cyclic citrullinated peptides (CCPs) influences the severity of rheumatoid arthritis. arthritis and rheumatism 50, 2113-2121.

van Gestel A M et al. (1996) Development and validation of the European League Against Rheumatism response criteria for rheumatoid arthritis. Comparison with the preliminary American College of Rheumatology and the World Health Organization/International League Against Rheumatism Criteria. arthritis and rheumatism 39, 34-40.

van Gestel A M et al. (1999) ACR and EULAR improvement criteria have comparable validity in rheumatoid arthritis trials. American College of Rheumatology European League of Associations for Rheumatology. The Journal of rheumatology 26, 705-711.

Wigersma L et al. (1991) [Extramural health care in AIDS: comparison between the United States and The Netherlands]. Nederlands tijdschrift voor geneeskunde 135, 1188-1191

Wong S H et al. (2009) Lactoferrin is a survival factor for neutrophils in rheumatoid synovial fluid. Rheumatology 48, 39-44.

Wu Z et al. (2005) Human neutrophil alpha-defensin 4 inhibits HIV-1 infection in vitro. FEBS letters 579, 162-166.

Yagi R et al. (2011) An updated view on transcription factor GATA3-mediated regulation of Th1 and Th2 cell differentiation. International immunology 23, 415-420.

Yamashita I I et al. (1999) Characterization of human and murine PMP20 peroxisomal proteins that exhibit antioxidant activity in vitro. The Journal of biological chemistry 274, 29897-29904.

Yang D et al. (2003) Eosinophil-derived neurotoxin (EDN), an antimicrobial protein with chemotactic activities for dendritic cells. Blood 102, 3396-3403.

Yang D et al. (2004) Human ribonuclease A superfamily members, eosinophil-derived neurotoxin and pancreatic ribonuclease, induce dendritic cell maturation and activation. Journal of immunology 173, 6134-6142.

Zhang J et al. (2002) Identification and characterization of a novel member of olfactomedin-related protein family, hGC-1, expressed during myeloid lineage development. genes 283, 83-93.

Zughaier S M et al. (2013) Peripheral monocytes derived from patients with cystic fibrosis and healthy donors secrete NGAL in response to Pseudomonas aeruginosa infection. Journal of investigative medicine : the official publication of the American Federation for Clinical Research 61, 1018-1025.

TABLE 1
Clinical and laboratory diagnostic data for RA patients before and during treatment with MTX.

| | Duration of | MTX treatment | | | | Swollen | Painful |
|---|---|---|---|---|---|---|---|
| Patient | disease | duration | Age | | RF | joints | joints |
| ID | (months) | (months) | (years) | Gender | (IU) | (28 basis) | (28 basis) |
| R_22+ | 8.8 | 0 | 27 | f | 617 | 10 | 13 |
| | | 3.8 | | | | 0 | 0 |
| R_23+ | 1.1 | 0 | 66 | m | 275 | 8 | 7 |
| | | 3.7 | | | | 0 | 1 |
| R_25+ | 0.6 | 0 | 44 | m | 305 | 6 | 8 |
| | | 3.7 | | | | 0 | 1 |
| R_39+ | 0.0 | 0 | 37 | m | 3 | 12 | 4 |
| | | 3.0 | | | | 0 | 0 |
| R_41+ | 0.1 | 0 | 77 | m | 8 | 9 | 15 |
| | | 5.9 | | | | 0 | 0 |
| R_44+ | 11.8 | 0 | 43 | f | 389 | 3 | 4 |
| | | 3.5 | | | | 0 | 2 |
| R_205+ | 1.1 | 0 | 35 | m | 72 | 6 | 10 |
| | | 3.7 | | | | 3 | 0 |
| R_206+ | 5.3 | 0 | 59 | m | 39 | 13 | 15 |
| | | 3.7 | | | | 2 | 0 |
| R_211+ | 1.4 | 0 | 47 | m | 41 | 7 | 7 |
| | | 3.7 | | | | 2 | 3 |
| R_221+ | 0.7 | 0 | 68 | f | 129 | 6 | 9 |
| | | 3.8 | | | | 2 | 2 |
| R_223+ | 0.0 | 0 | 70 | f | 45 | 6 | 8 |
| | | 3.5 | | | | 0 | 2 |
| R_1014+ | 12.3 | 0 | 52 | m | 318 | 4 | 9 |
| | | 3.7 | | | | 0 | 0 |
| R_1019+ | 1.1 | 0 | 37 | f | 35 | 1 | 1 |
| | | 3.0 | | | | 0 | 1 |
| R_1020+ | 2.8 | 0 | 63 | f | 234 | 2 | 12 |
| | | 3.0 | | | | 0 | 0 |
| R_28− | 1.4 | 0 | 59 | m | 12 | 6 | 8 |
| | | 3.7 | | | | 0 | 0 |
| R_31− | 2.2 | 0 | 61 | m | 1 | 9 | 9 |
| | | 3.7 | | | | 3 | 1 |
| R_42− | 0.3 | 0 | 22 | f | 14 | 11 | 22 |
| | | 4.9 | | | | 0 | 1 |
| R_43− | 0.0 | 0 | 45 | f | 81 | 0 | 3 |
| | | 3.0 | | | | 0 | 0 |
| R_45− | 0.0 | 0 | 65 | m | 58 | 2 | 9 |
| | | 3.0 | | | | 0 | 1 |
| R_47− | 6.0 | 0 | 32 | f | 85 | 4 | 6 |
| | | 3.03 | | | | 7 | 0 |
| R_202− | 2.2 | 0 | 57 | f | 28 | 8 | 8 |
| | | 3.8 | | | | 0 | 0 |
| R_204− | 4.1 | 0 | 54 | f | 3,040 | 8 | 9 |
| | | 3.9 | | | | 2 | 1 |
| R_215− | 0.8 | 0 | 73 | m | 48 | 15 | 25 |
| | | 3.7 | | | | 0 | 0 |
| R_217− | 0.0 | 0 | 75 | m | 53 | 16 | 18 |
| | | 3.7 | | | | 0 | 0 |
| R_218− | 2.6 | 0 | 20 | f | 14 | 6 | 13 |
| | | 3.6 | | | | 1 | 1 |
| R_1012− | 0.7 | 0 | 47 | m | 20 | 6 | 10 |
| | | 3.5 | | | | 1 | 1 |
| MR+_32 | 0.2 | 0 | 40 | m | 239 | 19 | 21 |
| | | 3.6 | | | | 12 | 14 |
| MR+_33 | 2.2 | 0 | 60 | f | 11 | 13 | 14 |
| | | 3.7 | | | | 7 | 3 |
| MR+_34 | 0.6 | 0 | 70 | f | 3 | 24 | 27 |
| | | 3.7 | | | | 2 | 6 |
| MR+_203 | 0.7 | 0 | 43 | f | 243 | 21 | 28 |
| | | 3.7 | | | | 8 | 5 |
| MR+_209 | 0.1 | 0 | 58 | f | 247 | 9 | 12 |
| | | 3.7 | | | | 1 | 5 |
| MR+_212 | 1.9 | 0 | 23 | f | 121 | 11 | 13 |
| | | 3.7 | | | | 8 | 14 |
| MR+_214 | 5.2 | 0 | 53 | f | 269 | 8 | 11 |
| | | 3.8 | | | | 2 | 5 |
| MR+_1011 | 42.1 | 0 | 74 | f | 366 | 8 | 14 |
| | | 4.2 | | | | 4 | 10 |
| MR+_1015 | 240.9 | 0 | 56 | f | 131 | 17 | 24 |
| | | 3.2 | | | | 4 | 18 |
| MR−_29 | 1.2 | 0 | 44 | f | 859 | 16 | 21 |
| | | 3.8 | | | | 0 | 3 |
| MR−_30 | 0.2 | 0 | 66 | m | 6 | 16 | 27 |
| | | 3.7 | | | | 0 | 10 |
| MR−_35 | 0.9 | 0 | 66 | f | 5 | 8 | 8 |
| | | 3.7 | | | | 0 | 4 |
| MR−_1017 | 20.6 | 0 | 72 | f | 278 | 0 | 15 |
| | | 3.0 | | | | 0 | 2 |
| NR_24+ | 2.4 | 0 | 67 | f | 26 | 7 | 7 |
| | | 3.7 | | | | 1 | 9 |
| NR_27+ | 1.2 | 0 | 53 | f | 104 | 8 | 12 |
| | | 3.7 | | | | 8 | 12 |
| NR_36+ | 1.1 | 0 | 68 | f | 34 | 0 | 4 |
| | | 8.9 | | | | 6 | 5 |
| NR_37+ | 2.8 | 0 | 77 | f | 639 | 2 | 0 |
| | | 4.0 | | | | 11 | 19 |
| NR_38+ | 3.8 | 0 | 64 | m | 1 | 4 | 7 |
| | | 6.0 | | | | 4 | 7 |
| NR_46+ | 0.0 | 0 | 70 | f | 45 | 1 | 5 |
| | | 2.6 | | | | 1 | 6 |
| NR_26− | 0.1 | 0 | 57 | f | 22 | 8 | 12 |
| | | 3.7 | | | | 8 | 12 |
| NR_40− | 0.5 | 0 | 58 | f | 83 | 5 | 14 |
| | | 3.5 | | | | 5 | 25 |
| NR_219− | 0.7 | 0 | 48 | f | 1,010 | 8 | 10 |
| | | 3.7 | | | | 0 | 12 |
| NR_1013− | 0.9 | 0 | 52 | f | 2 | 8 | 13 |
| | | 3.5 | | | | 7 | 16 |
| NR_1016− | 0.5 | 0 | 42 | m | 66 | 3 | 4 |
| | | 2.8 | | | | 1 | 6 |
| NR_1018− | 4.2 | 0 | 52 | f | 49 | 5 | 2 |
| | | 3.2 | | | | 6 | 5 |
| NR_1021− | 5.7 | 0 | 35 | m | 69 | 4 | 9 |
| | | 2.7 | | | | 0 | 13 |

| | CRP | | ANA | ACCPA | | | | |
|---|---|---|---|---|---|---|---|---|
| Patient | (mg/ | BSR | titer | titer | | | DAS28 | EULAR |
| ID | dl) | 1 h | (IU) | (IU) | HAQ | DAS28 | reduction | response |
| R_22+ | 1.64 | 35 | 640 | 622 | 1.6 | 6.410 | | |
| | 0.15 | 10 | | | 0.6 | 2.279 | 4.131 | R |
| R_23+ | 1.53 | 72 | 0 | 94 | 1.5 | 5.933 | | |
| | 0.07 | 10 | | | 0.8 | 2.351 | 3.581 | R |
| R_25+ | 3.70 | 33 | 320 | 604 | 1.4 | 5.821 | | |
| | 0.39 | 2 | | | 1.3 | 1.424 | 4.397 | R |
| R_39+ | 3.19 | 35 | 0 | 331 | nd | 5.009 | | |
| | 0.17 | 10 | | | nd | 1.612 | 3.397 | R |
| R_41+ | 3.17 | 54 | 320 | 26 | nd | 6.220 | | |
| | 0.56 | 8 | | | nd | 1.740 | 4.480 | R |
| R_44+ | 0.21 | 26 | 320 | 34 | nd | 4.735 | | |
| | 0.5 | 6 | | | nd | 2.323 | 2.411 | R |
| R_205+ | 1.96 | 20 | 320 | 1,000 | 0.9 | 5.556 | | |
| | 0.27 | 6 | | | 0.3 | 2.073 | 3.483 | R |
| R_206+ | 2.92 | 4 | 160 | 1,000 | 1.8 | 5.209 | | |
| | 0.34 | 4 | | | 0.0 | 1.429 | 3.780 | R |
| R_211+ | 1.83 | 28 | 160 | 54 | 1.0 | 5.745 | | |
| | 0.11 | 6 | | | 0.1 | 2.844 | 2.901 | R |
| R_221+ | 0.80 | 41 | 160 | 28 | 1.4 | 6.039 | | |
| | 0.40 | 10 | | | 0.1 | 2.841 | 3.198 | R |
| R_223+ | 0.10 | 32 | 320 | 7 | 1.4 | 5.714 | | |
| | 0.20 | 10 | | | 1.0 | 2.723 | 2.991 | R |
| R_1014+ | 0.98 | 36 | 80 | nd | nd | 5.167 | | |
| | 0.18 | 12 | | | nd | 2.165 | 3.002 | R |
| R_1019+ | 0.76 | 34 | 80 | 61 | nd | 3.307 | | |
| | 0.50 | 5 | | | nd | 1.966 | 1.342 | R |
| R_1020+ | 0.13 | 22 | 320 | 243 | nd | 5.624 | | |
| | 0.13 | 10 | | | nd | 1.754 | 3.870 | R |
| R_28− | 1.70 | 36 | 80 | 8 | 0.9 | 5.285 | | |
| | 0.50 | 11 | | | 0.1 | 1.750 | 3.536 | R |
| R_31− | 1.20 | 20 | 0 | 1 | 1.3 | 5.296 | | |
| | 3.00 | 2 | | | 0.5 | 1.688 | 3.607 | R |
| R_42− | 1.78 | 35 | 0 | 31 | nd | 6.886 | | |
| | 1.15 | 18 | | | nd | 3.146 | 3.740 | R |
| R_43− | 0.27 | 20 | 160 | 8 | nd | 3.626 | | |
| | 0.11 | 6 | | | nd | 1.396 | 2.230 | R |
| R_45− | 0.58 | 30 | nd | >1000 | nd | 4.873 | | |
| | nd | 2 | | | nd | 1.182 | 3.691 | R |
| R_47− | 6 | 23 | nd | >1000 | 0.75 | 4.648 | | |
| | 2 | 8 | | | 0.75 | 2.491 | 2.157 | R |
| R_202− | 0.39 | 48 | 80 | 9 | 1.9 | 5.935 | | |
| | 0.16 | 18 | | | 0.0 | 2.165 | 3.770 | R |
| R_204− | 0.75 | 34 | 2,560 | 957 | 0.9 | 5.604 | | |
| | 0.84 | 16 | | | 1.3 | 3.167 | 2.437 | R |
| R_215− | 3.55 | 49 | 320 | 6 | 2.5 | 7.948 | | |
| | 0.31 | 13 | | | 1.1 | 2.363 | 5.585 | R |
| R_217− | 3.11 | 73 | 160 | 11 | 2.1 | 7.431 | | |
| | 0.52 | 25 | | | 1.1 | 2.637 | 4.795 | R |
| R_218− | 1.54 | 81 | 80 | 3 | 0.4 | 6.241 | | |
| | 0.30 | 19 | | | 0.3 | 2.957 | 3.285 | R |
| R_1012− | 1.03 | 12 | 80 | 6 | nd | 5.042 | | |
| | 0.10 | 2 | | | nd | 1.608 | 3.434 | R |
| MR+_32 | 6.70 | 62 | 320 | 1,000 | 1.0 | 7.806 | | |
| | 3.70 | 20 | | | 1.0 | 5.796 | 2.010 | MR |
| MR+_33 | 0.87 | 26 | 0 | 18 | 0.1 | 6.361 | | |
| | 0.72 | 32 | | | 0.4 | 4.976 | 1.385 | MR |
| MR+_34 | 0.99 | 28 | 160 | 6 | 2.0 | 7.687 | | |
| | 0.33 | 18 | | | 1.1 | 4.210 | 3.477 | MR |
| MR+_203 | 1.67 | 68 | 80 | 581 | 2.5 | 8.328 | | |
| | 0.36 | 38 | | | 1.3 | 5.457 | 2.871 | MR |
| MR+_209 | 3.91 | 50 | 320 | 1,000 | 1.4 | 6.422 | | |
| | 1.53 | 48 | | | 0.6 | 4.533 | 1.889 | MR |
| MR+_212 | 2.67 | 42 | 80 | 490 | 0.0 | 6.610 | | |
| | 0.28 | 12 | | | 0.1 | 5.159 | 1.451 | MR |
| MR+_214 | 3.33 | 36 | 80 | 9 | 0.9 | 6.047 | | |
| | 0.95 | 18 | | | 0.3 | 3.907 | 2.140 | MR |
| MR+_1011 | 2.88 | 45 | 640 | 1,000 | nd | 6.823 | | |
| | 1.04 | 35 | | | nd | 5.380 | 1.443 | MR |
| MR+_1015 | 2.67 | 55 | 320 | 375 | nd | 7.831 | | |
| | 0.47 | 15 | | | nd | 5.812 | 2.019 | MR |
| MR−_29 | 0.47 | 91 | 640 | 434 | 1.9 | 7.788 | | |
| | 0.31 | 29 | | | 1.4 | 3.546 | 4.243 | MR |
| MR−_30 | 3.85 | 81 | 0 | 9 | 2.4 | 8.260 | | |
| | 0.31 | 26 | | | 0.5 | 4.348 | 3.912 | MR |
| MR−_35 | 2.50 | 33 | 1,280 | 4 | 0.9 | 5.076 | | |
| | 0.37 | 23 | | | 0.9 | 3.660 | 1.416 | MR |
| MR−_1017 | 0.42 | 40 | 5,120 | 27 | nd | 5.726 | | |
| | 0.37 | 35 | | | nd | 4.126 | 1.600 | MR |
| NR_24+ | 1.24 | 69 | 0 | 10 | 2.0 | 5.979 | | |
| | 1.47 | 72 | | | 1.5 | 5.823 | 0.156 | NR |
| NR_27+ | 5.40 | 41 | 320 | 3 | 0.0 | 5.908 | | |
| | 1.60 | 28 | | | 0.0 | 5.612 | 0.295 | NR |
| NR_36+ | 0.27 | 32 | 160 | 651 | nd | 4.246 | | |
| | 0.22 | 42 | | | nd | 5.831 | −1.585 | NR |
| NR_37+ | 0.32 | 55 | 1280 | 1000 | nd | 3.917 | | |
| | 1.13 | 45 | | | nd | 7.304 | −3.387 | NR |
| NR_38+ | 0.13 | 18 | 80 | 10 | nd | 4.486 | | |
| | 0.32 | 12 | | | nd | 4.202 | 0.284 | NR |
| NR_46+ | 3.81 | 36 | nd | 12 | nd | 4.743 | | |
| | nd | 24 | | | nd | 4.578 | 0.165 | NR |
| NR_26− | 1.70 | 19 | −1 | 3 | 1.9 | 5.696 | | |
| | 0.80 | 17 | | | 1.9 | 5.334 | 0.362 | NR |
| NR_40− | 0.11 | 24 | 160 | 26 | nd | 5.788 | | |
| | 0.11 | 35 | | | nd | 7.035 | −1.246 | NR |
| NR_219− | 1.57 | 49 | 320 | 58 | 1.5 | 6.475 | | |
| | 0.71 | 69 | | | 2.3 | 5.596 | 0.879 | NR |
| NR_1013− | 0.92 | 55 | 160 | 7 | nd | 6.745 | | |
| | 1.13 | 24 | | | nd | 5.906 | 0.839 | NR |
| NR_1016− | 1.00 | 26 | 320 | 9 | nd | 4.877 | | |
| | 0.16 | 29 | | | nd | 4.285 | 0.592 | NR |
| NR_1018− | 0.15 | 26 | 0 | 16 | nd | 4.127 | | |
| | 0.15 | 30 | | | nd | 4.744 | −0.617 | NR |
| NR_1021− | 0.50 | 24 | 80 | 48 | nd | 5.026 | | |
| | 0.12 | 30 | | | nd | 5.092 | −0.066 | NR |

ACPA = Anti-Citrullinated Protein Antibodies;
ANA = Antinuclear antibodies;
CRP = C-Reactive Protein;
DAS28 = Disease Activity Score (28 joints);
BSR = blood sedimentation rate;
f = female,
m = male;
HAQ = Health Assessment Questionnaire;
+ = HLA-DRB3-positive;
− = HLA-DRB3-negative;
ID = Patient Identification no.;
I.U. = International Unit;
n.d. = not determined;
MTX = Methotrexate;
R = responder,
MR = medium responder,
NR = non-responder

TABLE 2
Predictive genes for the HLA-DRB4-negative patient sub-group

| SEQ ID | | | | | Change | Change |
|---|---|---|---|---|---|---|
| NO. | Affymetrix ID | Gene symbol | Ref. seq. ID | Fold change | increase [%] | decrease [%] |
| 1 | 207269_at | DEFA4 | NM_001925 | 2.50 | 70.24 | 17.86 |
| 2 | 205382_s_at | CFD | NM_001928 | 2.15 | 73.81 | 11.90 |
| 3 | 205513_at | TCN1 | NM_000355 | 1.97 | 75.00 | 9.52 |
| 4 | 206111_at | RNASE2 | NM_002934 | 1.84 | 77.38 | 4.77 |
| 5 | 208699_x_at | TKT(*L1) | NM_001064 | 1.80 | 80.96 | 9.52 |
| 6 | 242457_at | unknown | Hs.674648 (*) | 1.72 | 70.24 | 14.29 |
| 7 | 202336_s_at | PAM | NM_000919 | 1.71 | 72.62 | 9.52 |
| 8 | 222922_at | KCNE3 | NM_0054 72 | 1.63 | 73.81 | 1.19 |
| 9 | 212468_at | SPAG9 | NM_001130527 (**) | 1.55 | 71.43 | 3.57 |
| 10 | 1560587_s_at | PRDX5 | NM_012094 | 1.41 | 71.43 | 1.19 |
| 11 | 39248_at | AQP3 | NM_004925 | −1.53 | 7.14 | 75.00 |
| 12 | 209604_s_at | GATA3 | NM_001002295 | −1.57 | 1.19 | 73.81 |
| 13 | 228949_at | WLS | NM_001002292 | −1.65 | 19.05 | 71.43 |
| 14 | 213757_at | EIF5A | NM_001143 760 | −1.69 | 2.38 | 73.81 |
| 15 | 215169_at | SLC35E2 | NM_001199787 | −1.73 | 72.62 | 72.62 |
| 16 | 225155_at | SNHG5 | NR_003038 | −1.74 | 76.19 | 76.19 |

(*) Sequence in sequence protocol: contig sequence of 5 EST mRNA sequences
(**) Sequence in sequence protocol: transcript variant 1, 2, 3 and 4 mRNA (NM_001130527.2 (variant 2)

TABLE 3
Predictive genes of the HLA-DRB4-negative patient sub-group

| SEQ ID | | | | | Change | Change |
|---|---|---|---|---|---|---|
| NO. | Affymetrix ID | Gene symbol | Ref. seq. ID | Fold change | increase [%] | decrease [%] |
| 17 | 226248_s_at | KIAA1324 | NM_020775 | 2.80 | 72.62 | 16.67 |
| 18 | 232365_at | SIAH1 | NM_001006610 | 1.72 | 71.43 | 7.14 |
| 19 | 201360_at | CST3 | NM_000099 | 1.54 | 70.24 | 2.38 |
| 20 | 224724_at | SULF2 | NM_001161841 | 1.52 | 75.00 | 1.31 |
| 21 | 212946_at | KIAA0564 | NM_001009814 | −1.51 | 0 | 71.43 |
| 22 | 236140_at | GCLM | NM_002061 | −1.52 | 0 | 73.81 |
| 23 | 200998_s_at | CKAP4 | NM_006825 | −1.55 | 3.57 | 71.43 |
| 24 | 209485_s_at | OSBPL1A | NM_001242508 | −1.65 | 3.57 | 70.24 |
| 25 | 235518_at | SLC8A1 | NM_001112800 | −1.94 | 8.33 | 72.62 |
| 26 | 227474_at | LOC654433 | NR_015377.2 | −2.17 | 10.71 | 71.43 |
| 27 | 206177_s_at | ARG1 | NM_000045 | −2.66 | 15.48 | 70.24 |
| 28 | 212531_at | LCN2 | NM_005564 | −3.09 | 19.05 | 73.81 |
| 29 | 207802_at | CRISP3 | NM_001190986 | −3.36 | 11.90 | 73.81 |
| 30 | 202018_s_at | LTF | NM_001199149 | −4.42 | 15.48 | 73.81 |
| 31 | 212768_s_at | OLFM4 | NM_006418 | −4.72 | 14.29 | 76.19 |
| 32 | 231688_at | MMP8 | NM_002424 | −6.04 | 11.90 | 80.95 |
| 33 | 209728_at | HLA-DRB4 | NM_021983.4 | | | |

TABLE 4
HLA-DRB4 signals obtained for the Affymetrix U133
Plus 2 microarray analyses

| | |
|---|---|
| HLA-DRB4-positive | HLA-DRB4-negative |
| RA sub-group | RA sub-group |

| Patient ID | Signal | Patient ID | Signal |
|---|---|---|---|
| NR_24+ | 1683.5 | NR_1013− | 6.3 |
| NR_27+ | 2179.4 | NR_1016− | 9.4 |
| NR_36+ | 2697.4 | NR_1018− | 10.1 |
| NR_37+ | 3367.1 | NR_1021− | 5.4 |
| NR_38+ | 3895.8 | NR_219− | 27.1 |
| NR_46+ | 4612.4 | NR_26− | 5.6 |
| MR_1011+ | 3291.4 | NR_40− | 6.6 |
| MR_1015+ | 1880.0 | MR_1017− | 20.5 |
| MR_203+ | 1997.3 | MR_29− | 44.3 |
| MR_209+ | 2061.2 | MR_30− | 5.0 |
| MR_212+ | 1413.7 | MR_35− | 31.0 |
| MR_214+ | 1673.4 | R_1012− | 39.1 |
| MR_32+ | 1433.3 | R_202− | 16.3 |
| MR_33+ | 1326.3 | R_204− | 8.3 |
| MR_34+ | 1420.3 | R_215− | 7.3 |
| R_1014+ | 1900.5 | R_217− | 39.6 |
| R_1019+ | 1888.1 | R_218− | 19.4 |
| R_1020+ | 2438.5 | R_28− | 19.8 |
| R_205+ | 2513.8 | R_31− | 3.3 |
| R_206+ | 4994.8 | R_42− | 36.8 |
| R_211+ | 1441.5 | R_43− | 4.5 |
| R_22+ | 1817.4 | R_45− | 63.9 |
| R_221+ | 3764.3 | R_47− | 38.2 |
| R_223+ | 2202.0 | | |
| R_23+ | 2167.7 | | |
| R_25+ | 2006.2 | | |
| R_39+ | 3592.3 | | |
| R_41+ | 4047.6 | | |
| R_44+ | 4048.4 | | |

Table 4 shows the signals for the sample set (209728_at) of the Affymetrix evaluations within the HLA-DRB4-positive (+) and the HLA-DRB4-negative RA patients sub-groups consisting of n=29 and n=23 patients of the responders (R), moderate responders (MR) and the non-responders (NR).

TABLE 5
qPCR validation of the predictive genes in (A) HLA-DRB4-positive and (B) HLA-DRB4-negative patient sub-groups.

| | | | | Correlation of microarray and qPCR data | | | | | |
|---|---|---|---|---|---|---|---|---|---|
| | | | | Pearson correlation | | Spearman correlation | | | |
| | FC | | | p value | Correlation | | Correlation | | FC |
| Gene | qPCR | Std. E. | 95% C.I. | qPCR | coefficient | p value | coefficient | p value | array |

(A)

| | | | | | | | | | |
|---|---|---|---|---|---|---|---|---|---|
| ARG1 | 1.7 | 0.32-10.74 | 0.07-29.02 | 0.292 | 0.68 | 0.000 | 0.79 | 0.000 | 2.7 |
| CKAP4 | 1.7 | 0.64-4.44 | 0.36-10.43 | 0.087 | 0.78 | 0.000 | 0.79 | 0.000 | 1.6 |
| CRISP3 | 3.9 | 0.46-45.76 | 0.06-183.20 | 0.070 | 0.42 | 0.001 | 0.51 | 0.000 | 3.4 |
| CST3 | 1.1 | 0.29-3.99 | 0.13-8.88 | 0.903 | 0.19 | 0.188 | 0.21 | 0.133 | −1.5 |
| GCLM | 1.4 | 0.70-2.82 | 0.48-5.05 | 0.188 | 0.30 | 0.031 | 0.34 | 0.015 | 1.5 |
| KIAA0564 | 2.1 | 0.71-6.48 | 0.41-28.51 | 0.036 | 0.41 | 0.003 | 0.22 | 0.129 | 1.5 |
| KIAA1324 | 1.1 | 0.05-39.70 | 0.01-197.08 | 0.896 | 0.49 | 0.000 | 0.52 | 0.000 | −2.8 |
| LCN2 | 4.0 | 1.08-14.58 | 0.28-26.27 | 0.007 | 0.84 | 0.000 | 0.89 | 0.000 | 3.1 |
| LTF | 3.4 | 0.23-56.21 | 0.11-359.63 | 0.244 | 0.39 | 0.005 | 0.41 | 0.003 | 4.4 |
| MMP8 | 7.7 | 1.53-45.23 | 0.13-170.97 | 0.008 | 0.76 | 0.000 | 0.83 | 0.000 | 6.0 |
| OLFM4 | 13.4 | 1.26-108.43 | 0.29-304.01 | 0.005 | 0.78 | 0.000 | 0.91 | 0.000 | 4.7 |
| OSBPL1A | 1.4 | 0.24-8.78 | 0.08-29.34 | 0.542 | 0.38 | 0.005 | 0.30 | 0.035 | 1.7 |
| SIAH1 | 1.2 | 0.57-2.49 | 0.26-4.45 | 0.499 | 0.19 | 0.191 | 0.16 | 0.257 | −1.7 |
| SLC8A1 | 2.2 | 0.85-6.40 | 0.53-15.64 | 0.010 | 0.47 | 0.001 | 0.31 | 0.025 | 1.9 |
| SULF2 | −1.2 | 0.353-1.697 | 0.25-4.32 | 0.455 | 0.68 | 0.000 | 0.68 | 0.000 | −1.5 |
| HLA-DRB4 | 1.5 | 0.71-3.04 | 0.50-5.72 | 0.118 | 0.75 | 0.000 | 0.87 | 0.000 | 0.8 |
| RPLP0 | 1.0 | | | | | | | | 1.0 |

(B)

| | | | | | | | | | |
|---|---|---|---|---|---|---|---|---|---|
| AQP3 | 1.6 | 0.68-3.42 | 0.46-6.20 | 0.067 | 0.60 | 0.000 | 0.60 | 0.000 | 1.5 |
| CFD | 1.1 | 0.25-3.79 | 0.08-7.41 | 0.829 | 0.26 | 0.065 | 0.12 | 0.387 | −2.2 |
| DEFA4 | −1.8 | 0.20-1.63 | 0.09-3.69 | 0.060 | 0.82 | 0.000 | 0.89 | 0.000 | −2.5 |
| EIF5A | 1.2 | 0.69-2.14 | 0.41-4.14 | 0.292 | −0.01 | 0.965 | −0.01 | 0.921 | 1.7 |
| GATA3 | 1.4 | 0.65-3.03 | 0.42-5.72 | 0.143 | 0.37 | 0.007 | 0.29 | 0.041 | 1.6 |
| KCNE3 | 1.4 | 0.51-5.12 | 0.11-10.01 | 0.326 | 0.10 | 0.478 | −0.06 | 0.680 | −1.6 |
| PAM | −1.2 | 0.09-7.41 | 0.02-40.43 | 0.809 | 0.39 | 0.004 | 0.44 | 0.001 | −1.7 |
| PRDX5 | −1.0 | 0.53-1.74 | 0.35-5.26 | 0.873 | 0.57 | 0.000 | 0.49 | 0.000 | −1.5 |
| RNASE2 | −1.3 | 0.36-1.35 | 0.22-2.17 | 0.141 | 0.59 | 0.000 | 0.63 | 0.000 | −1.8 |
| SLC35E2 | 1.7 | 0.54-5.37 | 0.23-10.21 | 0.132 | 0.49 | 0.000 | 0.50 | 0.000 | 1.7 |
| SNHG5 | 1.9 | 0.78-4.69 | 0.40-9.48 | 0.023 | 0.66 | 0.000 | 0.68 | 0.000 | 1.7 |
| SPAG9 | 1.3 | 0.40-4.61 | 0.13-11.28 | 0.449 | 0.07 | 0.618 | −0.05 | 0.710 | −1.6 |
| TCN1 | −1.9 | 0.01-30.77 | 0.01-167.35 | 0.522 | 0.20 | 0.157 | 0.25 | 0.078 | −2.0 |
| TKT | 2.1 | 0.16-25.57 | 0.04-79.12 | 0.274 | 0.12 | 0.390 | 0.10 | 0.506 | −1.8 |
| WLS | 1.2 | 0.39-3.67 | 0.12-8.38 | 0.660 | 0.61 | 0.000 | 0.67 | 0.000 | 1.7 |
| RPLP0 | 1.0 | | | | | | | | 1.0 |

Testing of the Affymetrix-based results for the differential gene expression of 30 of the 32 defined biomarkers was carried out with an independent method using quantitative Real Time PCR. RPLP0 was used as the reference gene. The table contains the gene expression differences for the RT-qPCR expressed as FC, the standard error expressed as Std. Error, the confidence interval expressed as C.I. and the corresponding probability values expressed as the p value qPCR to differentiate MTX-responders from non-responders using the REST analysis software (Pfaffl et al, 2002). For comparison purposes, the gene expression differences for the preceding microarray analyses are given. The correlation of the results for the individual genes from the microarray and RT-qPCR comparison was carried out using SPSS software using the Pearson or Spearman criteria.

1-13. (canceled)

14. A method for forecasting whether a patient will be a responder or a non-responder to treatment with MTX (methotrexate), comprising the steps of
(i) providing a patient sample,
(ii) detecting at least one mRNA biomarker selected from
ARG1, CKAP4, CRISP3, CST3, GCLM, KIAA0564, KIAA1324, LCN2, LOC654433/PAX8-AS1, LTF, OLFM4, OSBPL1A, MMP8, SIAH1, SLC8A1/BF223010, and SULF2
and/or selected from
AQP3, CFD, DEFA4, EIF5A, GATA3, Hs.674648, KCNE3, PAM, PRDX5, RNASE2, TCN1, TKT, SLC35E2, SNHG5, SPAG9, and WLS in combination with HLA-DRB4
in the patient sample,
and
(iii) assaying the relative expression level of the at least one mRNA biomarker and of
HLA-DRB4 by comparison with one or more reference standards and/or control samples,
wherein the patient is classified as a responder or non-responder based on said relative expression level.

15. The method as claimed in claim 14, wherein the treatment with MTX comprises a combination of biologics and MTX.

16. The method as claimed in claim 14, wherein the classification of the patient is carried out prior to commencing treatment with MTX (methotrexate).

17. The method as claimed in claim 14, wherein the sample is pre-selected into either a HLA-DRB4-positive or a HLA-DRB4-negative sample.

18. The method as claimed in claim 14, wherein the sample undergoes a pre-treatment comprising removing globin mRNA, reverse transcription of the total mRNA and/or labelling with a label.

19. The method as claimed in claim 14, wherein the detection in step (ii) comprises assaying for the presence of the mRNA marker and its expression levels,
or comprises assaying for the presence of an miRNA marker.

20. The method as claimed in claim 19, wherein the assay is carried out using
serial analysis of gene expression (SAGE), real time quantitative PCR (qPCR), bead technology, blot, RNA or next-generation sequencing, and/or
in-situ hybridization, Northern blot, DNA microarrays or DNA macroarrays.

21. The method as claimed in claim 14, wherein the patient to be treated has an inflammatory disease, chronic inflammatory disease, autoimmune disease and/or tumor disease.

22. The method as claimed in claim 21, wherein the inflammatory, chronic inflammatory, or autoimmune disease is
selected from rheumatoid arthritis (RA), primary chronic polyarthritis, juvenile idiopathic arthritis, systemic lupus erythematosus (SLE), systemic sclerosis (scleroderma), polymyositis, dermatomyositis, inclusion body myositis, psoriasis, multiple sclerosis, uveitis, Crohn's disease, Churg-Strauss syndrome (CSS), Boeck's disease, Bechterew's disease, relapsing polychondritis, colitis ulcerosa, polymyalgia rheumatica, giant cell arteritis, vasculitis, and myositides; and
the tumor disease is selected from: acute lymphatic leukemia (ALL) (juvenile and adult), transitional cell carcinoma of the bladder, breast cancer, medulloblastoma, ependymoma (juvenile and adult), non-Hodgkins lymphoma (NHL) (juvenile and adult), and osteosarcoma (juvenile and adult).

23. The method as claimed in claim 14, wherein at least 50% of the mRNA biomarker genes are detected in combination with HLA-DRB4.

24. The method as claimed in claim 14, wherein in the case of treatment of rheumatoid arthritis (RA), all 32 biomarker genes (100%) are assayed in combination with HLA-DRB4.

25. The method as claimed in claim 14, wherein the sample is selected from whole blood, peripheral blood leukocytes and purified blood cells.

26. The method as claimed in claim 14, wherein in step (ii), at least one mRNA biomarker(s) selected from
CKAP4, CRISP3, KIAA0564, LCN2, OLFM4, MMP8, and SLC8A1/BF223010
and/or selected from
AQP3, DEFA4, or and SNHG5 is detected in combination with HLA-DRB4 in the patient sample.

27. The method as claimed in claim 14, wherein in step (iii), standard samples containing one or more housekeeping genes and control samples are samples of responders and/or non-responders.

28. A kit for forecasting whether a patient will be a responder or a non-responder to treatment with MTX (methotrexate), comprising
(a) implementing means for detecting at least one mRNA biomarker selected from
ARG1, CKAP4, CRISP3, CST3, GCLM, KIAA0564, KIAA1324, LCN2, LOC654433/PAX8-AS1, LTF, OLFM4, OSBPL1A, MMP8, SIAH1, SLC8A1/BF223010, and SULF2
and/or selected from
AQP3, CFD, DEFA4, EIF5A, GATA3, Hs.674648, KCNE3, PAM, PRDX5, RNASE2, TCN1, TKT, SLC35E2, SNHG5, SPAG9, and WLS
in combination with HLA-DRB4
in a patient sample,
(b) at least one reference standard comprising a sample containing one or more housekeeping genes, and
(c) at least one control sample comprising a sample from a responder and/or a non-responder.

29. The kit as claimed in claim 28, wherein the implementing means for detecting at least one mRNA biomarker detects a biomarker selected from
CKAP4, CRISP3, KIAA0564, LCN2, OLFM4, MMP8, and SLC8A1/BF223010
and/or selected from
AQP3, DEFA4, and SNHG5.

30. The kit as claimed in claim 28, wherein the implementing means for detecting at least one mRNA biomarker in a patient sample comprises:
arrays, chips,
primers,
marker and labels.


SEQUENCE LISTING (40 sequences)

SEQ ID NO: 1 - DNA, 542 bp, Homo sapiens
gtctgccctctctgctcgccctgcctagcttgaggatctgtcaccccagccatgaggattatcgccctcctcgctgctattctcttggtagccctccaggtccgggcaggcccactccaggcaagaggtgatgaggctccaggccaggagcagcgtgggccagaagaccaggacatatctatttcctttgcatgggataaaagctctgctcttcaggtttcaggctcaacaaggggcatggtctgctcttgcagattagtattctgccggcgaacagaacttcgtgttgggaactgcctcattggtggtgtgagtttcacatactgctgcacgcgtgtcgattaacgttctgctgtccaagagaatgtcatgctgggaacgccatcatcggtggtgttagcttcacatgcttctgcagctgagcttgcagaatagagaaaaatgagctcataatttgctttgagagctacaggaaatggttgtttctcctatactttgtccttaacatctttcttgatcctaaatatatatctcgtaacaag

SEQ ID NO: 2 - DNA, 1173 bp, Homo sapiens
gtgtctcagccacagcggcttcaccatgcacagctgggagcgcctggcagttctggtcctcctaggagcggccgcctgcgcggcgccgccccgtggtcggatcctgggcggcagagaggccgaggcgcacgcgcggccctacatggcgtcggtgcagctgaacggcgcgcacctgtgcggcggcgtcctggtggcggagcagtgggtgctgagcgcggcgcactgcctggaggacgcggccgacgggaaggtgcaggttctcctgggcgcgcactccctgtcgcagccggagccctccaagcgcctgtacgacgtgctccgcgcagtgccccacccggacagccagcccgacaccatcgaccacgacctcctgctgctacagctgtcggagaaggccacactgggccctgctgtgcgccccctgccctggcagcgcgtggaccgcgacgtggcaccgggaactctctgcgacgtggccggctggggcatagtcaaccacgcgggccgccgcccggacagcctgcagcacgtgctcttgccagtgctggaccgcgccacctgcaaccggcgcacgcaccacgacggcgccatcaccgagcgcttgatgtgcgcggagagcaatcgccgggacagctgcaagggtgactccgggggcccgctggtgtgcgggggcgtgctcgagggcgtggtcacctcgggctcgcgcgtttgcggcaaccgcaagaagcccgggatctacacccgcgtggcgagctatgcggcctggatcgacagcgtcctggcctagggtgccggggcctgaaggtcagggtcacccaagcaacaaagtcccgagcaatgaagtcatccactcctgcatctggttggtctttattgagcacctactatatgcagaaggggaggccgaggtgggaggatcattggatctcaggagttcgagatcagcatgggccacgtagcgcgactccatctctacaaataaataaaaaattagctgggcaattggcgggcatggaggtgggtgcttgtagttccagctactcaggaggctgaggtgggaggatgacttgaacgcaggaggctgaggctgcagtgagttgtgattgcaccactgccctccagcctgggcaacagagtgaaaccttgtctctctctacaaaaaaaaaaaaaaaa

SEQ ID NO: 3 - DNA, 2104 bp, Homo sapiens
tacctccaggagggctgctctgcccttccttcctctgttctttggccttatgttccccgccaccacagaccttcccccgccccacccctctgcagacttagccgtgcattgcaggcatggaggattaatcagtgacaggaagctgcgtctctcggagcggtgaccagctgtggtcaggagagcctcagcagggccagccccaggagtctttcccgattcttgctcactgctcacccacctgctgctgccatgaggcaccttggggccttcctcttccttctgggggtcctgggggccctcactgagatgtgtgaaataccagagatggacagccatctggtagagaagttgggccagcacctcttaccttggatggaccggctttccctggagcacttgaaccccagcatctatgtgggcctacgcctctccagtctgcaggctgggaccaaggaagacctctacctgcacagcctcaagcttggttaccagcagtgcctcctagggtctgccttcagcgaggatgacggtgactgccagggcaagccttccatgggccagctggccctctacctgctcgctctcagagccaactgtgagtttgtcaggggccacaagggggacaggctggtctcacagctcaaatggttcctggaggatgagaagagagccattgggcatgatcacaagggccacccccacactagctactaccagtatggcctgggcattctggccctgtgtctccaccagaagcgggtccatgacagcgtggtggacaaacttctgtatgctgtggaacctttccaccagggccaccattctgtggacacagcagccatggcaggcttggcattcacctgtctgaagcgctcaaacttcaaccctggtcggagacaacggatcaccatggccatcagaacagtgcgagaggagatcttgaaggcccagacccccgagggccactttgggaatgtctacagcaccccattggcattacagttcctcatgacttcccccatgcgtggggcagaactgggaacagcatgtctcaaggcgagggttgctttgctggccagtctgcaggatggagccttccagaatgctctcatgatttcccagctgctgcccgttctgaaccacaagacctacattgatctgatcttcccagactgtctggcaccacgagtcatgttggaaccagctgctgagaccattcctcagacccaagagatcatcagtgtcacgctgcaggtgcttagtctcttgccgccgtacagacagtccatctctgttctggccgggtccaccgtggaagatgtcctgaagaaggcccatgagttaggaggattcacatatgaaacacaggcctccttgtcaggcccctacttaacctccgtgatggggaaagcggccggagaaagggagttctggcagcttctccgagaccccaacaccccactgttgcaaggtattgctgactacagacccaaggatggagaaaccattgagctgaggctggttagctggtagcccctgagctccctcatcccagcagcctcgcacactccctaggcttctaccctccctcctgatgtccctggaacaggaactcgcctgaccctgctgccacctcctgtgcactttgagcaatgccccctgggatcaccccagccacaagcccttcgagggccctataccatggcccaccttggagcagagagccaagcatcttccctgggaagtctttctggccaagtctggccagcctggccctgcaggtctcccatgaaggccaccccatggtctgatgggcatgaagcatctcagactccttggcaaaaaacggagtccgcaggccgcaggtgttgtgaagaccactcgttctgtggttggggtcctgcaagaaggcctcctcagcccgggggctatggccctgaccccagctctccactctgctgttagagtggcagctccgagctggttgtggcacagtagctggggagacctcagcagggctgctcagtgcctgcctctgacaaaattaaagcattgatggcctgtggacctgcaaaaaaaaaaaaaaaaaaaaaaaaaaaaaaaa

SEQ ID NO: 4 - DNA, 735 bp, Homo sapiens
gctgcccctgaaccccagaacaaccagctggatcagttctcacaggagctacagcgcggagactgggaaacatggttccaaaactgttcacttcccaaatttgtctgcttcttctgttggggcttctggctgtggagggctcactccatgtcaaacctccacagtttacctgggctcaatggtttgaaacccagcacatcaatatgacctcccagcaatgcaccaatgcaatgcaggtcattaacaattatcaacggcgatgcaaaaaccaaaatactttccttcttacaacttttgctaacgtagttaatgtttgtggtaacccaaatatgacctgtcctagtaacaaaactcgcaaaaattgtcaccacagtggaagccaggtgcctttaatccactgtaacctcacaactccaagtccacagaatatttcaaactgcaggtatgcgcagacaccagcaaacatgttctatatagttgcatgtgacaacagagatcaacgacgagaccctccacagtatccggtggttccagttcacctggatagaatcatctaagctcctgtatcagcactcctcatcatcactcatctgccaagctcctcaatcatagccaagatcccatctctccatatactttgggtatcagcatctgtcctcatcagtctccataccccttcagctttcctgagctgaagtgccttgtgaaccctgcaataaactgctttgcaaattc

SEQ ID NO: 5 - DNA, 2179 bp, Homo sapiens
gatccgagccccgcctcctccccctgccccgcctctcccatccccgccccgccccgcccggcgacttaacgcgcccccgccccgcgcccggcctcggcagccgcctgtcgccgcgggagcagccgctatctctgtgtgtccgcgtgtgcgcccggtccccgcctgccgcaccatggagagctaccacaagcctgaccagcagaagctgcaggccttgaaggacacggccaaccgcctacgtatcagctccatccaggccaccactgcggcgggctctggccaccccacgtcatgctgcagcgccgcagagatcatggctgtcctctttttccacaccatgcgctacaagtcccaggacccccggaatccgcacaatgaccgctttgtgctctccaagggccatgcagctcccatcctctacgcggtctgggctgaagctggtttcctggccgaggcggagctgctgaacctgaggaagatcagctccgacttggacgggcacccggtcccgaaacaagctttcaccgacgtggccactggctccctgggccagggcctcggggccgcttgtgggatggcctacaccggcaaatacttcgacaaggccagctaccgagtctattgcttgctgggagacggggagctgtcagagggctctgtatgggaggccatggccttcgccagcatctataagctggacaaccttgtggccattctagacatcaatcgcctgggccagagtgacccggccccactgcagcaccagatggacatctaccagaagcggtgcgaggccttcggttggcatgccatcatcgtggatggacacagcgtggaggagctgtgcaaggcctttggccaggccaagcaccagccaacagccatcattgccaagaccttcaagggccgagggatcacgggggtagaagataaggagtcttggcatgggaagcccctccccaaaaacatggctgagcagatcatccaggagatctacagccagatccagagcaaaaagaagatcctggcaacccctccacaggaggacgcaccctcagtggacattgccaacatccgcatgcccagcctgcccagctacaaagttggggacaagatagccacccgcaaggcctacgggcaggcactggccaagctgggccatgccagtgaccgcatcatcgccctggatggggacaccaaaaattccaccttctcggagatcttcaaaaaggagcacccggaccgcttcatcgagtgctacattgctgagcagaacatggtgagcatcgcggtgggctgtgccacccgcaacaggacggtgcccttctgcagcacttttgcagccttcttcacgcgggcctttgaccagattcgcatggccgccatctccgagagcaacatcaacctctgcggctcccactgcggcgtttccatcggggaagacgggccctcccagatggccctagaagatctggctatgtttcggtcagtccccacatcaactgtcttttacccaagtgatggcgttgctacagagaaggcagtggaactagccgccaatacaaagggtatctgcttcatccggaccagccgcccagaaaatgccatcatctataacaacaatgaggacttccaggtcggacaagccaaggtggtcctgaagagcaaggatgaccaggtgaccgttatcggggctggggtgaccctgcacgaggccttggccgctgccgaactgctgaagaaagaaaagatcaacatccgcgtgctggaccccttcaccatcaagcccctggacagaaaactcattctcgacagcgctcgtgccaccaagggcaggatcctcaccgtggaggaccattattatgaaggtggcattggtgaggctgtgtccagtgcagtagtgggcgagcctggcatcactgtcacccacctggcagttaaccgggtaccaagaagtgggaagccggctgagctgctgaagatgtttggtatcgacagggatgccattgcacaagctgtgaggggcctcatcaccaaggcctagggcgggtatgaagtgtggggcgggggtctatacattcctgagattctgggaaaggtgctcaaagatgtactgagaggaggggtaaatatatgttttgagaaaaatgaattggccctgaaaaaaaaaaaaaaaaaa

SEQ ID NO: 6 - DNA, 658 bp, Homo sapiens
ccttcaggtacccgggaattcggccacggccgacatgttttttttttttgttttttggtagagacaggtcttgcttgctttattgctcaggctggtctcctgggctcaagcaatcctctcacctccgcctcttgaagtgctgggattagaggcgtgagccactgtgcctggccaagtattacattcttcacagaatgtactactgaatcattccttgggaatcaagaagaagaaataaatgcaagactaactttatgggcagaactctcagaatgaagagattcagaaggaggctgatagagaggaaccggtcatgtgtctcattatttattgtttattgataaggcataccgtcagagaaaaactgaaacagcactaacttctcaaggaaaatattcacgtatcactgttctgtctgaatgttataagtggttctactatattctttctgtaaagctgaacatagctggattatccttaagaaggatggatacatattcaaggaaaactttcatgaagcactaattgcattcatcacaataatagaagcattatttttacaatcaaattgtgtgagatattgaaaagctataaaagtgtggcataactatggaatgatgattatcactgtactttgaacttcatacatctccccttc

SEQ ID NO: 7 - DNA, 5359 bp, Homo sapiens
gttctgaatgatgactgacgcgggtttgggtgatacccctcacagcccctgtcattccggagtcataaggcacccgcgcgtctagccccagcgccagggcacgcgagcggcgctggagggaggaaagcttccgcctgcgggccggacaaaagtcccgcctgcccacggctttttgcccgccgctcgtgaccgagacgcctcgccgcggccagctcgctgctctcgctggcggatggtgtgtggccgccgcaggacgcccgccgtgcccgggccatgaagtagcggctgctggcggcgccgctgcccaaccgccagccccagccccgcgctgcgctgcccggtcctctcccggcggggtcgtatcggcgtggacatggctggccgcgtccctagcctgctagttctccttgtttttccaagcagctgtttggctttccgaagcccactttctgtctttaagaggtttaaagaaactaccagaccattttccaatgaatgtcttggtaccaccagacccgtagttcctattgattcatcagattttgcattggatattcgcatgcctggggttacacctaaacagtccgatacatacttctgcatgtctatgcgaataccagtggatgaggaagccttcgtgattgacttcaagcctcgagccagcatggatactgtccatcacatgttactttttggatgcaatatgccttcatccactggaagttactggttttgtgatgaaggaacctgtacagataaagccaatattctgtatgcctgggcgagaaatgctccccctacccggctccccaaaggtgttggattcagagttggaggagagactggaagtaaatactttgtactacaggtacactatggggatattagtgcttttagagataataacaaggactgttctggtgtgtccttacacctcacacgtctgccacagcctttaattgctggcatgtaccttatgatgtctgttgacactgttatcccagcaggagaaaaagtggtgaattctgacatttcatgccattataaaaattatccaatgcatgtctttgcctatagagttcacactcaccatttaggtaaggtagtaagtggatacagagtaagaaatggacagtggacactgattggacggcagagccctcagctgccacaggctttctaccctgtggggcatccagttgatgtaagttttggtgacctactggctgcaagatgtgtattcactggtgaaggaaggacagaagccacacacattggtggcacgtctagtgatgaaatgtgcaacttatacattatgtattacatggaagccaagcatgcagtttctttcatgacctgtacccagaatgtagctccagatatgttcagaaccataccaccagaggccaacattccaattcccgtgaagtctgatatggttatgatgcatgaacatcataaagaaacagaatataaagataagattcctttactacagcagccaaaacgagaagaagaagaagtgttagaccagggtgatttctattcactactttccaagctgctaggagaaagggaagatgttgttcatgtgcacaaatataatcctacagaaaaggcagaatcagagtcagacctggtagctgagattgcaaatgtagtccaaaaaaaggatcttggtcgatctgatgccagagagggtgcagaacatgagaggggtaatgctattcttgtcagagacagaattcacaaattccacagactagtatctaccttgaggccaccagagagcagagttttctcattacagcagcccccacctggtgaaggcacctgggaaccagaacacacaggagatttccacatggaagaggcactggattggcctggagtatacttgttaccaggccaggtttctggggtggctctagaccctaagaataacctggtgattttccacagaggtgaccatgtctgggatggaaactcgtttgacagcaagtttgtttaccagcaaataggactcggaccaattgaagaagacactattcttgtcatagatccaaataatgctgcagtactccagtccagtggaaaaaatctgttttacttgccacatggcttgagtatagataaagatgggaattattgggtcacagacgtggctctccatcaggtgttcaaactggatccaaacaataaagaaggccctgtattaatcctgggaaggagcatgcaaccaggcagtgaccagaatcacttctgtcaacccactgatgtggctgtggatccaggcactggagccatttatgtatcagatggttactgcaacagcaggattgtgcagttttcaccaagtggaaagttcatcacacagtggggagaagagtcttcagggagcagtcctctgccaggccagttcactgttcctcacagcttggctcttgtgcctcttttgggccaattatgtgtggcagaccgggaaaatggtcggatccagtgttttaaaactgacaccaaagaatttgtgagagagattaagcattcatcatttggaagaaatgtatttgcaatttcatatataccaggcttgctctttgcagtgaatgggaagcctcattttggggaccaagaacctgtacaaggatttgtgatgaacttttccaatggggaaattatagacatcttcaagccagtgcgcaagcactttgatatgcctcatgatattgttgcatctgaagatgggactgtgtacattggagatgctcataccaacaccgtgtggaagttcaccttgactgagaaattggaacatcgatcagttaaaaaggctggcattgaggtccaggaaatcaaagaagccgaggcagttgttgaaaccaaaatggagaacaaacccacctcctcagaattgcagaagatgcaagagaaacagaaactgatcaaagagccaggctcgggagtgcctgttgttctcattacaacccttctggttattccggtggttgtcctgctggccattgccatatttattcggtggaaaaaatcaagggcctttggagcagattctgaacacaaactcgagacgagttcaggaagagtactgggaagatttagaggaaagggaagtggaggcttaaaccttggtaatttctttgcaagccgtaagggctacagtcgaaaagggtttgaccggcttagcactgagggcagtgaccaagagaaagaggatgatggaagtgaatcagaagaggagtattcagcacctctgcctgcgctcgcaccttcctcctcctgaaaaccaagctttgatttagattgagtaagatttacccagaatgtcagattcctttccctttagcacgtttaaagttctgtgtatttaattgtaaactgtactagtctgtgtgggactgtacacactttatttacttcgttttggttaagttggcttctgtttctagttgaggagtttcctaaaagttcataacagtgccattgtctttatatgaacatagactagagaaaccgtcctctttttccatcataattctaatctaacaatggaagatttgcccatttacacttttgagactttttggtggatgtaaataaccccattctttgcttgaacacagtattttcccaatagcactttcattgccagtgtctttctttggtgcctttcctgttcagcattcttagcctgtggcagtaaagagaaactttgtgctacatgacgacaaagctgctaaatctcctatttttttaaaatcactaacattatattgcaatgaaggaaataaaaaagtctctatttaaattcttttttaaattttcttcagttggtgtgtttttgggatgtcttatttttagatggttacactgttagaacactattttcagaatctgaatgtaatttgtgtaataaagtgttttcagagcattagctgtcagtgtattttccagtttttgcgtatttgcagattttacatacaacttttataataattacacaaacccacaaatattagtgaaacttactcgatgtcttcaactaaaagaaatgtgtgtattgtacaaaatttagaagatactttagccaatataaattaaaaaccagcctgagtttacataaatttgtaaagtcaggctcttctaaaatccaaagagggtttttgcctatatatcaatcagaggataaatactttaataaaaggtaatcacagctaagtggatacctgtgtctcaaattacatatgcaaatgatccatcagtagggatcactaataatagttttccttttaaaaaataatttcagggcaggtacagtggctcaagcctatacttccaggactttgggaggccaagcagaaggattgtgggaggccaagcagaaggattgtttgagcccagaaattcaaggctgcagtgagctatgatcaatccactgtactccagcctaggctacagagtgagatcctttctctaaaataaaatacaatttccatgtatccataggaatatattcatcttttacagtgattgcagattagttttaaaacgtctttttcgtaattcgtcaatgcaagttagtaagaaccagataattttccattttaaaatgataggaatctaaaactctttttcaaaaatgccaacctgtttttccggcatcatagtagttggaataatacagatataattgactaatcataaagtacatgagagtacagaagggaaatttggaaacggtaagtctgctagggcattacaatcctgtcctagcactccacctttattctgccaacctgggttaattaaagatggtagcctggaaagtaatgaatgacattgacttcaggcaatctttccactgattattcttggctgaacttcatttatcgagtcagagaatgcactgcctgagaaatgtcccagaggagtgaatcctaggcctggccacagtagaatcgcctggagaatttaagaaaaaaattgttgggcactcactctttccagtgatctttatttagttggcttacaatggaatacaggtatggttcatgtttttaaatttgtccaggtgtttctattgtgcagtcacagttgaaaaccattgtcttcgagaatagctattctatcttgccagttacaataagtggatagctatattttcacataaattatagtttacagatgtttggagggggaagacaggatctgaggtgttttgatatgactgttagcaccaaaatctgaatgccttaattgttgaatgtgttaaattggataattaaaatgggcataaatgacttattaaaaaagcaaagggaaaaaaaaaaaaaaaaaaaaaaaa

SEQ ID NO: 8 - DNA, 3070 bp, Homo sapiens
gagcccagagccagagagcgcgctgggcggtgctgggcacccgcggagtggaacggggctggtggaatgcacagggtcgcagcgcttgggccaccctcggtcagagggcgccgtgtccagcgagcaaacgggcgccccggagccttgctgagaggcagctctgggctttcccagctccgaagtcaatactgagatcccagatgtgtccagagacatcctgaagaggctcgggggtggaggagccttagtgtgtccacaaagggactcctgaaactgactgagagccagtggatttgccagcagtctgagcttctaccgagtcttcccccacctcaatccctgttgctatggagactaccaatggaacggagacctggtatgagagcctgcatgccgtgctgaaggctctaaatgccactcttcacagcaatttgctctgccggccagggccagggctggggccagacaaccagactgaagagaggcgggccagcctacctggccgtgatgacaactcctacatgtacattctctttgtcatgtttctatttgctgtaactgtgggcagcctcatcctgggatacacccgctcccgcaaagtggacaagcgtagtgacccctatcatgtgtatatcaagaaccgtgtgtctatgatctaacacgagagggctgggacggtggaagaccaagacacctggggattgcgtctggggcctccagaactctgctgtggactgcatcaggtctcagtgtccctatctgtaagatcaacaagaaacacggttaagggaggtcgtcactggggtgggagaagaggggctggtagaccgaagccttgtgcataaggattttttcccaggaaaagatagactttataaacagtgggagcccatgaacaaacatataaaagtagcaacagataatgaccaataactggttcagtggctggagtattaggggcctggggattggagaacggagaagaagttgtagcagagggaaatgagacaggaagatgctctggggacacattttttatgtgttatcttcagccatgagaagcagtgatgactatcccatatcacagatatgatttaccaccaccaccctgcccccgctcccgtgaagaaagcagggcaagtgctgtgctgcccatttgggcctgcatagtgccatgattggaacccaggaactctggtctccttgcctagtgcttttcaaaactctgtgctacacaggagtggatccaggcctgaaggtcatacaattctggggactctctttaagaaaaagaattctaaaatatcttacttttgcaaacattatgaaaatatactgccacattaatatgttgctagggcccctgctaggaccttaagaaggagctcatgtgagtcaggaccctgaatgttaggcctcgttagctctatggttcatatgcttcttgaaccaagtcacagggcacttcccagccacattgccaggcaacaggactaaactacctccaaagcaagcagtcttttcagttttgactgagtgatgtgagaaacttcttttcttttcttttctttttttttttttgagacagtctccctatgtcacccaggctgtggtgcagcaacccaatcttggctcactgcaacccccacctcccgggttcaagcaattatcctgcctcagccacctgagtagctgggattacaggttcctgtcaccacacccagttaatttatatatatatatatatatatatatttaagtagagacagggtttcacatgttgcccaggctggtctcgaactcctgtcctcaagttatctgcccattttggtctcccaaagtgctgggattacaagtgtaagccaccacgactatctgagagaagttttctgatgtcatgttgaatctgcttctaaaagactgatactgccaaggtgggcggatcacctgaggtcaggagttcgagaccagcctggccaacatggtgaaaccccatctactaaaaaaatacaaaaattagccagacctggtggcgggtgcccgtattcccagctacttgggaggctgaggcaggagaattgtttgaacccgggaggtggaggttgcagtaagccaagatcacgccactgcactccagcctgggtgacagagcaaggctctgtctcaaaaaaaaacaaaaacaaaaacaaaaaagactgatatcgcacctaaattattattatattaaaagaagcagagtatgagagacaggtacatggtccagtaggaagagaagcagccctgattctaccacttaaggtgatgtatgatcttaggctggacacttctctccctcatccgttttcctcttcaacataatgaaatagacttgaaagtctctaaggctctatcagttctgacattctaggcttcatatacattaagttgagccatatgtaatcactgtgtttgtaggttagaaacagctgagtatcgtagtttcatatatggttccagctaatacatgcaatgtggctggtgaacacttctgaattcagaaactatcccagatctcagctagaaccatccactgttctgtttgtccagtttcaacttaagggatctccatgcggtccctggaagtacccattgaaacatgcgtatttgtgtatagcagaactctgaaataatattctgacagcagttatctctgaggaattgggttataggtgattttccctttccgcatgataaatttatgtaatatttgactgacttgaccgtaagtatgttacttgtataataaaaggaaaaaaggtacttctattttgaaaaaataaaaataaaagcctttgggttcttgaatggaggatcatggaacacatttgctgccatatgcagttatgttgatgctctgcaaacctgtgctgagccctgttgctcaagcccttcctcatctcttcttgagggagaaggtggagacttccttaaggagatgtgacatatgggaagacaacagattcagaaatttacgtggataggactttagacaccacccagcccaaacttccaaataaaatatggaacgcaa

SEQ ID NO: 9 - DNA, 8243 bp, Homo sapiens
agttggtgcagcggcggttggggtgagagcgcctacgccacccctcccctcctccggccccggcccccaccccgccgggcccagccccagccccggcccgggctccagccctagtacccgtcccagcccgagtcgggtccgtcccgtgcgggcaggtgccgcccctctgccggcgacgccccgggccgcccgcccgtccgcttgccaccatggagctggaggacggtgtggtgtatcaggaggagcccggcggctccggggccgtgatgtcggagcgggtgtccggcctggccggctccatctaccgcgagttcgagcggcttatcgggcgctatgacgaggaggtggtcaaagagctgatgccgctggtggtggctgtgctggagaacctggactcggtgttcgcgcaggaccaggagcaccaggtggagctggagctgctgcgggacgacaacgagcagctcatcacccagtacgagcgggagaaggcgctgcgcaagcacgctgaggagaaattcattgaatttgaagactctcaagaacaggaaaaaaaggacttacagacccgagtggaatctttagaatctcaaacaagacaacttgagctgaaagcgaaaaactatgctgaccagattagcagacttgaagaaagagaagcagaactgaagaaggaatataatgcattacatcaaagacacactgagatgatccataattatatggaacatttagaaagaacaaaacttcatcagctctcagggagtgatcaactagaatccacagctcatagtagaattagaaaagaacgccctatatcattaggaattttcccattacctgctggagatggattgcttacacctgatgctcagaaaggaggagagacccctggatctgagcaatggaaatttcaggaattaagtcaaccacgttctcataccagcctgaaggatgagctttctgatgttagccaaggcggatctaaagctaccactccagcatcaacagctaattcagatgtggcaacaattcctactgatactcccttaaaggaagaaaacgaaggatttgtgaaggttacagatgcgccaaataaatcagagataagcaaacacattgaagtacaggtagcccaggaaactagaaatgtatctactggctctgctgaaaatgaagaaaagtcagaagttcaagcaatcatcgaatctactcctgagctggatatggacaaagatctcagtggatataaaggttcaagcactcccaccaaaggcatagagaacaaagcttttgatcgcaatacagaatctctctttgaagaactgtcttcagctggctcaggcctaataggagatgtggatgaaggagcagatttactaggaatgggtcgggaagttgagaatcttatattagaaaatacacaactgttggaaaccaaaaatgctttgaacatagtgaagaatgatttgatagcaaaagtggatgaactgacctgtgagaaagatgtgctgcaaggggaattggaggctgtgaagcaagccaaactgaaactagaggaaaagaacagagaattggaggaagagcttaggaaagctcgggcagaagctgaagatgcaaggcaaaaagcaaaagatgacgatgatagtgatattcccacagcccagaggaaacggtttactagagtagaaatggcccgtgttctcatggagcgaaaccagtataaagagagattgatggagcttcaggaagctgttcgatggacagagatgattcgggcatcacgagaaaatccagccatgcaggaaaaaaaaaggtcaagcatttggcagtttgtgccaactcgtttcagccgacttttcagctcctcaagtaacacgactaagaagcctgaaccacctgttaatctgaagtacaatgcacccacgtctcatgttactccgtccgtcaagaaaagaagcagcaccttatctcagctccctggggataagtccaaagcctttgatttccttagtgaagaaactgaagctagtttagcctcacgcagagaacaaaagagagagcagtatcgtcaggtaaaagcacatgttcagaaggaagacggtagagtgcaggcttttggctggagtctgcctcagaagtacaaacaggtaaccaatggtcaaggtgaaaataagatgaaaaatttacctgtgcctgtctatctcagacctctggatgaaaaagatacatcaatgaagctgtggtgtgctgttggagtcaatttatctggtgggaagaccagagatggtggttctgttgttggagcaagtgtattttacaaggatgttgctggtttggatacagaaggcagtaaacagcgaagtgcctctcagagtagtttagataagttagatcaggaacttaaggaacagcagaaggagttaaaaaatcaagaagaattatccagtctagtttggatctgtaccagcactcattcggctacaaaagttcttattattgatgctgttcaacctggcaacatcctagacagtttcactgtttgcaactctcatgttctgtgcattgcaagtgtgccaggtgcacgagaaacagactaccctgcaggagaagatctttcagaatctggtcaggtagacaaagcatctttatgtggaagtatgacaagcaacagctcagcagagacagacagcctgttaggaggcatcacagtggttggttgttctgcagaaggtgtgacgggagctgccacttcccctagtacaaatggtgcttctccagtgatggataaaccaccagaaatggaagcagaaaatagtgaggttgatgaaaatgttccaacagcagaagaagcaactgaagctacagaagggaatgcggggtcagctgaagacacagtggacatctcccaaactggcgtctacacagagcatgtctttacagatcctttgggagttcagatcccagaagacctctccccagtgtatcagtcgagcaatgactcagatgcatataaagatcaaatatcagtactgccaaatgaacaagacttggtgagagaagaagcccagaaaatgagtagtcttttaccaactatgtggcttggagctcaaaatggctgtttgtatgtccattcatctgtagcccagtggaggaaatgtctccattccattaaacttaaagattcgattctcagtattgtacacgtgaagggaatcgtgttagtagccctggctgacggcacccttgcaatctttcacagaggagtggatgggcagtgggatttgtcaaactatcacctcttagaccttggacggcctcatcattccatccgttgcatgactgtggtacatgacaaagtctggtgtggctataggaacaaaatctatgtggtgcagccaaaggccatgaaaatagagaaatcttttgatgcacatcccaggaaggagagccaagtgcgacagcttgcgtgggtgggggatggcgtgtgggtctccattcgcttggattctacgctccgtctctatcatgcacacacttatcaacatctacaggatgtggacattgagccttatgtaagcaaaatgttaggtactggaaaactgggcttctcttttgtgagaattacagctcttatggtgtcttgtaatcgtttgtgggtggggacaggaaatggtgtcattatctccatcccattgacagaaacaaataaaacctcaggtgtaccaggaaatcgtcctggaagtgtaatccgtgtatatggtgatgaaaacagtgataaagtgactccagggacatttataccctattgttcaatggcacatgcacagctttgcttccatgggcaccgggatgctgtgaaattctttgtggcagtcccaggtcaagtcatcagcccacaaagtagcagtagtggcacggatctgacgggtgacaaagcagggccatctgcacaggagcctggtagtcagacgcccttgaagtctatgcttgtcatcagtggaggagagggctacatcgacttccgaatgggtgatgaaggtggagaatcagaacttcttggagaggatcttccacttgaaccttctgtcaccaaagcagaaaggagtcacttgatagtgtggcaagtgatgtatggcaatgagtgagcccatgggaaacaggtggagatggggaagccgtctcttctgcatggtttattttccctctatccttttatttaatgctcttttgtgagataagtttcaccacataatgtgtgagcattttttcctgttaactttatattacaaaatccgttctaccataacaatacagaggaactagctgtgttactgcaccagtgttataggtaacttcagtatattatgaacaaatcaaagaatgtttacttcctgcaaactggtgaattatagaaagcaatccagatgtggtttactctgccacagtctaatgtcattcacttcatttgatggggtcacttgttagctgtcactaataatggaattaatgggaaacacttgataaatgaaactgtaccgttaaatacaatagctgagttttccccagtgtattgtaaaattgacacacactaatacaagatggattatcaggatgtatctaaatgtcccgagagggttaaaagctaactgtaaattactttaactttcactttcaaatctgacaaatcttgtttatatggtatataaaactttgttttcatcagatttgggggggttttatattaaagaaattaagactacactatttaaataaacgtttcctgtttctgacttattagagctctaaagtttatgaggcctacttctctgaatattctgatgggttttttttgagaccagtctcattttcatactgacatgtctgaaagatttattatttataaagcttaggacatactctgggaagcttagaagatacttcagcactgggaggattttttaaaggaaatgtgttttgtatttcttgtaccagagaatggtgatgttgattgagttttttccatgtaaaaaatgtacttgtttgccaaatgttcctagattgctcttaacctgttaatacaagtaaaactgtagtggggatggagtctggcctaagtacagaatgagggatttaattagaaaagttattttcttttgttctgtatcaaactgcagaacagctatatacttggtattttgtacgtgaataacgttttagcttttgtgcccttttgagcttgaagggttatcctcaaaggggaaaactatgaaggggaagaagacaaacctaagataccataaagtaaaggttgacattacatattttcattcacaatgcccattaaaaaaaaaaaataggttgggcatggtggctcacgcctgtaatcccaacactttaggaggctgaggcagatggattgcttgagcccaggagttcaagaccagcctgggcaacatggcgaaaccctgtctctaccaaaaaaaaaaaacaaaataaattaaccgggtgtggtggcacatgcctgtagtcccagctacttgggaggctgaggtgggaggatgacctgggcccaggaggtggaggttgcagtgagccttgatagcaccactgcactccagcctgggtgacggagcaagaccctgtctcaaaacagacaaacaagcaaaaaatagattaaagtctggatttcactgattttcttgcttaataagttttttaaaaccacgatgctgcaattttttccctctcaagcttcttgaaaatgtgtgatttacccttttttatctattactctaggcaaagctaaatatgatttttttttttttttttgagacggagtctggctctgttgcccaggctggaatgcagtggtgcgatctcagctcactgcaacctccacctctcaggttcaagtagttctcctgcctcagcctcccaagtagctgggattacaggtgtgtgccaccctgcccagctaatttttgtatttttagtagagacagggtttcaccacattggccaggctggtctcaaactcctgacctcagatgatctacccgccttggcctcccgaagtgcttgaattacaggcgtgagcccccgcgccggtccgaagcaaagctaaatgtaattttatgtactgttttaacagtataaacctatgggataaaggcccccaatcatcagaaggactactgaaaagaaaagaaagcaaacgtagatgtcaaattgtctaaacattcttaagtaccactgattttttttttctggactattatacaaagatgtctgtaaaaagtataccagtggcttttatgcatatacacgattaggctagattgtgaagtacatgggatttcatgagccagaggaggcatttggatccattgggccacatgtgactataagcacattgtgtacacaataaaattgtagccactcatgtattaaaactgttttggtaaaagctgtattaaaagaataattttttgcttgagctacttagtcacttttttgcagggcaccaaaatgtaggctacttttccatagcttgtttgctatgtttttatttcatcttaaatctgagccatttttggcaatatcttttaaaaaatcattattaaacatttattggaaatgttgtatagattcacaagaaagagacaccaaagtgtttgggaattttaatcccaagaagtaaacatcccagagtcagatcccatttgctgttgttcagccatgtttgtatgactgaatgtaatttaggaaatcaggagttagagatgaaaaatacaaacatgtcaaatggttgtaattgctttttttgaacttactggctttaacttaagtaatataggtgtgtagttgttttatctaaagatgtcagattatctgtggccctgttaattaactgcagggaccagagagaggacaaaaatgcctgtataatttgctttaaaggcttgtccttatgtttgtaatttcagttaaatattttctacagattgcttcacttcccctttcccccaataacaagttcatattcttgtaatgtttactttggtcaacgtggtaatttttggaagaaaatgccaaaatgtaagcttctcacaagcagcaaaggaatgtaactgcctgtagctaccacatcaagggagagaagaggcttgcgtactttgggttgatctcactgtacaaaaaaagtgtacccctattggaagaaataaggaatacttgaaggtttctcctttttttacacttgagtcatgtgcaaggttgaaccatatgaaattgccagtgttcaaccattgctgatctgcaaaagcgacaatatcatatgcctcaacctaacacttactcttaaatgcaggttaaaatctctgtgaggcaaagggataatattctctaggaactcacttttgagagtatttttctctaacccatgtggatctaaattttaaaaataggaatgaatctcttaattgcagcttgtggagagtataaaacattttgagcacaatatgcagcacataacttttcagattgtgtttgcatagaaaaggttaaaatcaattctttcagttactgtattgttcatttcttgtaaaggtcttcctacatttgagacgagaccctttaacataggctgccttagtaacaaaataaaacagatgtgtcataatattaactgtcctaaagaacatgtgtcttgcaaatgccatggaactgaatatttatttccttgtgagtattatgaccgttaccaaaggactcaaccagatttagggctcttcttaattgatcatgttcagaaagggactcatttggtcccactttgtaacatgggaagagtagaagagaaaattaaatgtggaaatcataaatgcttttctaacagggttatgctattctgtaacactaaatgtctgtcttttctcttaaaagattaagttttattaattttgtcatacatttctctatttgaatatatcttaaccattttatgttccaaattcgttttttgtccaagacattctatcagaattttacttgcctctataatctgaaaaatgggttctagaatgtcccacttgctgtctcttagaggctgagcttcatttctatgagcaaagagctcatttagcaatgtagttatttcagtatttatttctattatggaatccctggggttcagaatgtaactttgtacatgagatatatataaatatgtatatgaacatgta

SEQ ID NO: 10 - DNA, 913 bp, Homo sapiens
cgcgcctgcgcagtggaggcggcccaggcccgccttccgcagggtgtcgccgctgtgccgctagcggtgccccgcctgctgcggtggcaccagccaggaggcggagtggaagtggccgtggggcgggtatgggactagctggcgtgtgcgccctgagacgctcagcgggctatatactcgtcggtggggccggcggtcagtctgcggcagcggcagcaagacggtgcagtgaaggagagtgggcgtctggcggggtccgcagtttcagcagagccgctgcagccatggccccaatcaaggtgggagatgccatcccagcagtggaggtgtttgaaggggagccagggaacaaggtgaacctggcagagctgttcaagggcaagaagggtgtgctgtttggagttcctggggccttcacccctggatgttccaagacacacctgccagggtttgtggagcaggctgaggctctgaaggccaagggagtccaggtggtggcctgtctgagtgttaatgatgcctttgtgactggcgagtggggccgagcccacaaggcggaaggcaaggttcggctcctggctgatcccactggggcctttgggaaggagacagacttattactagatgattcgctggtgtccatctttgggaatcgacgtctcaagaggttctccatggtggtacaggatggcatagtgaaggccctgaatgtggaaccagatggcacaggcctcacctgcagcctggcacccaatatcatctcacagctctgaggccctgggccagattacttcctccacccctccctatctcacctgcccagccctgtgctggggccctgcaattggaatgttggccagatttctgcaataaacacttgtggtttgcggccatctccttggttaaaaaaaaa

SEQ ID NO: 11 - DNA, 1882 bp, Homo sapiens
agcgctcctataaagggagccaccagcgctggaggccgctgctcgctgcgccaccgcctcccgccacccctgcccgcccgacagcgccgccgcctgccccgccatgggtcgacagaaggagctggtgtcccgctgcggggagatgctccacatccgctaccggctgctccgacaggcgctggccgagtgcctggggaccctcatcctggtgatgtttggctgtggctccgtggcccaggttgtgctcagccggggcacccacggtggtttcctcaccatcaacctggcctttggctttgctgtcactctgggcatcctcatcgctggccaggtctctggggcccacctgaaccctgccgtgacctttgccatgtgcttcctggctcgtgagccctggatcaagctgcccatctacaccctggcacagacgctgggagccttcttgggtgctggaatagtttttgggctgtattatgatgcaatctggcacttcgccgacaaccagctttttgtttcgggccccaatggcacagccggcatctttgctacctacccctctggacacttggatatgatcaatggcttctttgaccagttcataggcacagcctcccttatcgtgtgtgtgctggccattgttgacccctacaacaaccccgtcccccgaggcctggaggccttcaccgtgggcctggtggtcctggtcattggcacctccatgggcttcaactccggctatgccgtcaaccctgcccgggactttggcccccgcctttttacagcccttgcgggctggggctctgcagtcttcacgaccggccagcattggtggtgggtgcccatcgtgtccccactcctgggctccattgcgggtgtcttcgtgtaccagctgatgatcggctgccacctggagcagcccccaccctccaacgaggaagagaatgtgaagctggcccatgtgaagcacaaggagcagatctgagtgggcaggggccatctccccactccgctgccctggccttgagcatccactgactgtccaagggccactcccaagaagcccccttcacgatccaccctttcaggctaaggagctccctatctaccctcaccccacgagacagccccttcaggatttccactggaccttgcccaaatagcaccttaggccactgcccctaagctggggtggaaccggaatttgggtcaatacatccttttgtctcccaagggaagagaatgggcagcaggtatgtgtgtgtgtgcatgtgtgtgcatgtgtgtgcatgtgtgtgcaggggtgtgtgtgtgtggggggggttcccagatattcagggcaagggaccagtcggaagggattctggctattgggggagcccagagacaggggaaggcagcctgtccatctgtgcataaggagaggaaagttccagggtgtgtatgtttcaggggcttcacatggaggagctgcagatagatatgtgtttctgtgtatgtgtatgtctgcctttttttctaagtgggggcttctacaggcttttgggaagtagggtggatgtgggtagggctgggaggagggggccacagcttaggtttggagctctggatgtacatacataagtaggagcagtgggacgtgtttctgtcataatgcaggcatgaagggtggagtgaagtcaggtcataagtttcatgtttgcttttgttttgttttgtttttaatgtatgtagcagatgttacagtcttagggatccgggatgggagaccccactttagaaagggtcgtcactcctttaatcctctactcaacaatgtactcttttacttttatattaaaaaaaataaaataaatatgtgcctaaaacctccaaaaaaaaaaaa

SEQ ID NO: 12 - DNA, 3070 bp, Homo sapiens
ggcgccgtcttgatactttcagaaagaatgcattccctgtaaaaaaaaaaaaaaaatactgagagagggagagagagagagaagaagagagagagacggagggagagcgagacagagcgagcaacgcaatctgaccgagcaggtcgtacgccgccgcctcctcctcctctctgctcttcgctacccaggtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacagaaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttggatgatatttattaaatagcttctaagagtccggcggcatctgtcttgtccctattcctgcagcctgtgctgagggtagcagtgtatgagctaccagcgtgcatgtcagcgaccctggcccgacaggccacgtcctgcaatcggcccggctgcctcttcgccctgtcgtgttctgtgttagtgatcactgcctttaatacagtctgttggaataatattataagcataataataaagtgaaaatattttaaaactacaa

SEQ ID NO: 13 - DNA, 2069 bp, Homo sapiens
cgtccacaggcggcggagcggccacaatcacagctccgggcattgggggaacccgagccggctgcgccgggggaatccgtgcgggcgccttccgtcccggtcccatcctcgccgcgctccagcacctctgaagttttgcagcgcccagaaaggaggcgaggaaggagggagtgtgtgagaggagggagcaaaaagctcaccctaaaacatttatttcaaggagaaaagaaaaagggggggcgcaaaaatggctggggcaattatagaaaacatgagcaccaagaagctgtgcattgttggtgggattctgctcgtgttccaaatcatcgcctttctggtgggaggcttgattgggcccacaacggcagtgtcctacatgtcggtgaaatgtgtggatgcccgtaagaaccatcacaagacaaaatggttcgtgccttggggacccaatcattgtgacaagatccgagacattgaagaggcaattccaagggaaattgaagccaatgacatcgtgttttctgttcacattcccctcccccacatggagatgagtccttggttccaattcatgctgtttatcctgcagctggacattgccttcaagctaaacaaccaaatcagagaaaatgcagaagtctccatggacgtttccctggcttaccgtgatgacgcgtttgctgagtggactgaaatggcccatgaaagagtaccacggaaactcaaatgcaccttcacatctcccaagactccagagcatgagggccgttactatgaatgtgatgtccttcctttcatggaaattgggtctgtggcccataagttttaccttttaaacatccggctgcctgtgaatgagaagaagaaaatcaatgtgggaattggggagataaaggatatccggttggtggggatccaccaaaatggaggcttcaccaaggtgtggtttgccatgaagaccttccttacgcccagcatcttcatcattatggtgtggtattggaggaggatcaccatgatgtcccgacccccagtgcttctggaaaaagtcatctttgcccttgggatttccatgacctttatcaatatcccagtggaatggttttccatcgggtttgactggacctggatgctgctgtttggtgacatccgacagggcatcttctatgcgatgcttctgtccttctggatcatcttctgtggcgagcacatgatggatcagcacgagcggaaccacatcgcagggtattggaagcaagtcggacccattgccgttggctccttctgcctcttcatatttgacatgtgtgagagaggggtacaactcacgaatcccttctacagtatctggactacagacattggaacagagctggccatggccttcatcatcgtggctggaatctgcctctgcctctacttcctgtttctatgcttcatggtatttcaggtgtttcggaacatcagtgggaagcagtccagcctgccagctatgagcaaagtccggcggctacactatgaggggctaatttttaggttcaagttcctcatgcttatcaccttggcctgcgctgccatgactgtcatcttcttcatcgttagtcaggtaacggaaggccattggaaatggggcggcgtcacagtccaagtgaacagtgcctttttcacaggcatctatgggatgtggaatctgtatgtctttgctctgatgttcttgtatgcaccatcccataaaaactatggagaagaccagtccaatggaatgcaactcccatgtaaatcgagggaagattgtgctttgtttgtttcggaactttatcaagaattgttcagcgcttcgaaatattccttcatcaatgacaacgcagcttctggtatttgagtcaacaaggcaacacatgtttatcagctttgcatttgcagttgtcacagtcacattgattgtacttgtatacgcacacaaatacactcatttagcctttatctcaaaatgttaaatataaggaaaaaagcgtcaacaataaatattctttgagtattgtcttacttctcttaaaaaaaaaaaaaaaaaa

SEQ ID NO: 14 - DNA, 1256 bp, Homo sapiens
accacactagcgcgctagaagagtggggcggaaagacatctcccgcgcatgtgtggaactggggggactgattccaagacaaggcgcccaccccacagagcaagttttctgaaacgtttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccagggtggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtcttttttttttttttttttttaattcaatctggaatcagaaagcggtggattctggcaaatggtccttgtgccctccccactcatccctggtctggtcccctgttgcctatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaagcccccaa

SEQ ID NO: 15 - DNA, 2177 bp, Homo sapiens
tggctctggggcactgaggagcggcgcccgcggggcagcgaggagcccgatgcagggttctgcgcgtcatttccggtcccgcgggcgccccgtgaagcccacctggatccgccagcgctgtgccactccccagtgccgagctccgagctgtctccgcggcctcgcgcccggcccctccaccgcgcacctcttaggccccgcccgccagcgtccctttgttgtgaaggcgccggggcctagcgctatgcctgcggcggagactgcatcaggctctcgtcctcgggctccacccggggagctgtgcccagacagcagaagggaaggatgtcacttctgagatgaggttcagaaaggcctgggctcctgtcctggctgctctctcccactccctgatgagcttgctggatgaaagctcctgtcaggctgtggggcgtcctgtggagaagctggcaagaaactggtgggggccctttccacctatagccagcaaggaactgaacccagccagcgtccacctgagtgagctcagaggtgggcctcagccccagttgagccttcggatgaggtcacagccctggtccatggtgtgactgcagcttcaggagggaccttaagccagaggtgcctggctaagcttcccgcagattgctgccccacaggagccaacatcaaaagcatttgttgttttgaggtgcgtctgcttctacgctttgcctgggagaggccctggtggcctcgttcctggcgcccggagtccctgctgcggccccacccccgggcggtcacggtgacccatgctgcccagcctggaggtaaaatcgttcgtggctgtggcttcagcatgtcgtcctcggtgaaaaccccagcactggaagagctggttcctggctccgaagagaagccgaaaggcaggtcgcctctcagctggggctctctgtttggtcaccgaagtgagaagattgtttttgccaagagcgacggcggcacagatgagaacgtactgaccgtcaccatcacggagaccacggtcatcgagtcagacttgggtgtgtggagctcgcgggcgctgctctacctcacgctgtggttcttcttcagcttctgcacgctcttcctcaacaagtacatcctgtccctgctgggaggcgagcccagcatgctaggtgcggtgcagatgctgtccaccacggttatcgggtgtgtgaaaaccctcgttccttgctgtttatatcagcacaaggcccggctttcctacccacccaacttccttatgacgatgctgtttgtgggtctgatgaggtttgcaactgtggttttgggtttggtcagcctgaaaaatgtggcggtttcgtttgctgagacggtgaagagctccgcccccatcttcacggtgatcatgtctcggatgattctgggggagtacacaggacgtcccagtgatcgggaggagcgggaagagcttcagctacaaccaggacgtggtgctgctgcttctgacagacggagtcctgttccaccttcagagcgtcacggcgtacgccctcatggggaaaatctccccggtgactttcaggcccagtaaatgcggatgtttatcttccattgtttgtttcctgagattcagatgtctaaagcatttttctgtgacttttcaaggcaagaggaaaacctgacatatggaaagggaattaattgctcgttttatcctcttctcctgcaatgctctgaatccatgggtttggagtggggccctgggagttgggggaagcaccatacccagtgagtctgcactttgaggacccactgccagtgtcagcttcaaaatcacattgtaaaaggcccggcgcggtggctcacgcctgtaatcccagcactttaggaggacgaagcaggcggatcacttgaggtcaggagttcgagaccagcctggccaatatggcgaaacctcgtctctaccaaaaatataaaaattagccgggcgtggtggcgcgggcctgtggttccagctactcgggaggctgaggcaggaaaattccttgaacccgggaggaggctgcagtgagccaagaccacgccactgcactccaacctgggcggcagagcaaggctccgactcaaaagtaaatacatgaataaataaaataaaatcacatcgtaaaaaaaaaaaaaaaaaaaaa

SEQ ID NO: 16 - DNA, 524 bp, Homo sapiens
cttttacgtcggccttcgcgagcgtctgggcgggtggtaggaacaatggcgctgtcttcagtggcacagtggagcagctctgaagatgcaaagatacacgaaaaaacttccagaacatctgggagaatatttaatggaaaatcgcttggttaaaacctgacacttttaacagtgaacagcgttctgagtgtggacgagtagccagtgaagataatgaatgtcgaatgtgactgactagcagcttcattttgaatgagggtcgctgtctgcccattgatagaggccagattgtcttggaagttccaaagttgcaacgatttctggctagtgccacgaggtttacttgactgttgtgtgaaaagctgataagaaaaccatccagaaaaaagctcttcgttttacaaacatgaaaataaaacatgtaattttggattatgttcctttttgttattacttttaaataggtcctgaaataacatggggagcattaaatggaaaatccactaaaaaaaaaaaaaaaaaaa

SEQ ID NO: 17 - DNA, 7054 bp, Homo sapiens
agagaggaggcggttgtcaaggcgacgtgggtaggaggagaggacagagggaggaggaaggatgggcggtgttggcgtagccgcagggaggtgactgaagcgagcctggcctcttgcatcctccgcctgtgtacctccctcccctttttttccgccttctgccagcagaagcagcagccgcagcacctgagccgctactgccgctcactcaggacaacgctatggctgagcctgggcacagccaccatctctccgccagagtcaggggaagaactgagaggcgcataccccggctgtggcggctgctgctctgggctgggaccgccttccaggtgacccagggaacgggaccggagcttcatgcctgcaaagagtctgagtaccactatgagtacacggcgtgtgacagcacgggttccaggtggagggtcgccgtgccgcataccccgggcctgtgcaccagcctgcctgaccccgtcaagggcaccgagtgctccttctcctgcaacgccggggagtttctggatatgaaggaccagtcatgtaagccatgcgctgagggccgctactccctcggcacaggcattcggtttgatgagtgggatgagctgccccatggctttgccagcctctcagccaacatggagctggatgacagtgctgctgagtccaccgggaactgtacttcgtccaagtgggttccccggggcgactacatcgcctccaacacggacgaatgcacagccacactgatgtacgccgtcaacctgaagcaatctggcaccgttaacttcgaatactactatccagactccagcatcatctttgagtttttcgttcagaatgaccagtgccagcccaatgcagatgactccaggtggatgaagaccacagagaaaggatgggaattccacagtgtggagctaaatcgaggcaataatgtcctctattggagaaccacagccttctcagtatggaccaaagtacccaagcctgtgctggtgagaaacattgccataacaggggtggcctacacttcagaatgcttcccctgcaaacctggcacgtatgcagacaagcagggctcctctttctgcaaactttgcccagccaactcttattcaaataaaggagaaacttcttgccaccagtgtgaccctgacaaatactcagagaaaggatcttcttcctgtaacgtgcgcccagcttgcacagacaaagattatttctacacacacacggcctgcgatgccaacggagagacacaactcatgtacaaatgggccaagccgaaaatctgtagcgaggaccttgagggggcagtgaagctgcctgcctctggtgtgaagacccactgcccaccctgcaacccaggcttcttcaaaaccaacaacagcacctgccagccctgcccatatggttcctactccaatggctcagactgtacccgctgccctgcagggactgaacctgctgtgggatttgaatacaaatggtggaacacgctgcccacaaacatggaaacgaccgttctcagtgggatcaacttcgagtacaagggcatgacaggctgggaggtggctggtgatcacatttacacagctgctggagcctcagacaatgacttcatgattctcactctggttgtgccaggatttagacctccgcagtcggtgatggcagacacagagaataaagaggtggccagaatcacatttgtctttgagaccctctgttctgtgaactgtgagctctacttcatggtgggtgtgaattctaggaccaacactcctgtggagacgtggaaaggttccaaaggcaaacagtcctatacctacatcattgaggagaacactaccacgagcttcacctgggccttccagaggaccacttttcatgaggcaagcaggaagtacaccaatgacgttgccaagatctactccatcaatgtcaccaatgttatgaatggtgtggcctcctactgccgtccctgtgccctagaagcctctgatgtgggctcctcctgcacctcttgtcctgctggttactatattgaccgagattcaggaacctgccactcctgccccactaacacaattctgaaagcccaccagccttatggtgtccaggcctgtgtgccctgtggtccagggaccaagaacaacaagatccactctctgtgctacaacgattgcaccttctcacgcaacactccgaccaggactttcaactacaacttctccgctttggcaaacactgtcactcttgctggagggccaagcttcacttccaaagggctgaaatacttccatcactttaccctcagtctctgtggaaaccagggtaggaaaatgtctgtgtgcaccgacaatgtcactgacctccggattcctgagggtgagtcagggttctccaaatctatcacagcctacgtctgccaggcagtcatcatccccccagaggtgacaggctacaaggccggggtttcctcacagcctgtcagccttgctgatcgacttattggggtgacaacagatatgactctggatggaatcacctccccagctgaacttttccacctggagtccttgggaataccggacgtgatcttcttttataggtccaatgatgtgacccagtcctgcagttctgggagatcaaccaccatccgcgtcaggtgcagtccacagaaaactgtccctggaagtttgctgctgccaggaacgtgctcggatgggacctgtgatggctgcaacttccacttcctgtgggagagcgcggctgcttgcccgctctgctcagtggctgactaccatgctatcgtcagcagctgtgtggctgggatccagaagactacttacgtgtggcgagaacccaagctatgctctggtggcatttctctgcctgagcagagagtcaccatctgcaaaaccatagatttctggctgaaagtgggcatctctgcaggcacctgtactgccatcctgctcaccgtcttgacctgctacttttggaaaaagaatcaaaaactagagtacaagtactccaagctggtgatgaatgctactctcaaggactgtgacctgccagcagctgacagctgcgccatcatggaaggcgaggatgtagaggacgacctcatctttaccagcaagaagtcactctttgggaagatcaaatcatttacctccaagaggactcctgatggatttgactcagtgccgctgaagacatcctcaggaggcctagacatggacctgtgagaggcactgcctgcctcacctgcctcctcaccttgcatagcacctttgcaagcctgcggcgatttgggtgccagcatcctgcaacacccactgctggaaatctcttcattgtggccttatcagatgtttgaatttcagatctttttttatagagtacccaaaccctcctttctgcttgcctcaaacctgccaaatatacccacactttgtttgtaaattatgcccttgcttgtatcttgtttcccaaaatggcccatccgccagagccatagcttcgtctgctcataattcttatagctttggaatgaaaatatttctatcttcttaagtatagaaactatttcctctgtcctctaacttaagggcagaaacagctgggagttttcctcgcatgccctcagctcatgatctcttcaggagagaggctgggtgaggagggtgtcggggttccctggtggataatcttcatagcagcctggatccatttcccctggataaccagctcaaagggagtgaaaatggtagtctgagggcaaggggagcaaggcctgggtaagaaaagccttgaaaagcataaaaagaggccgggcgcggtggctcacgcctgtaatcccagcactttgggaggccgaggcgggcagatcatgaggtcgggagattgagaccatcctggctaacacggtgaaaccccgtctctactaaaaatacaaaaaattagccgggcgtggtggcgggtgcctgtagtcccagctactcgggaggctgaggcgggagaatagcgtgaacctggaaggcggagcttgcagtgagccgagatcgcgccactgcactccatccagcctgggtgacagagtgagactctgcctcaaaaaaaaaaaaaaaaaaaaagaaaagcacaaagagaggcaacaaggaatgtttttgtttttgagacaggctctcactctgtcacctaggctggagtgcagtggcataatcactgttcagtgcagcctcaagctcttgggctcaagctatcctcccatctcaacctctcaagtagctaggactacaagtgtgcaccaccaggctcactaatttttatattttttgtagacacagggtttcaccatgttgcccaggctggtctccaactcctgggctcaagtgatctgtccgcctcagcctcccaaactgctgggattacaggcataagccactgcactcagccttttatttgttttttaaaccacgtagctcattgccttctcttaagtaaatgatagatattctcactgaagccaaaggaataagttcatcaagaaaatgcccaaagccctggtggatacatcctccctatcttttttttaaaccttccactatcactctatgacactgaaaagaaccaggtaagccccaaacccagatgttccagccttatcctctgttgggtttacccacagacatagcaaaccctgtcagtgaggaaaattccccatccttgagtgcccccgtcctagaagtttgggccatattatggaacaggggtctcttatttgaaaagagcacaaggaggccaagattttaatggggcactttaggggatacagcccacaatggcatgggcctgaggtggccgtgatgtctgcttctaagcttaacgcatctgctcaggcacagaataaacgtctaggctggccaaaaaaggaactgaatcccaggcccatacgccagcaccagaatcaaaccagtcttcaaggaaggaaggctaggagagtttaacaagattttcactgggcccagcatggtggctcacacctgtaatcccaaggcagaatggtggcttgagctcaggagttcaagaccagcctgggcaacacagtgagaccctgtctctaaaaaatttaaaaataaacaaggtgttcaccaagctgggatacttctcactattaagcccctatctttctctttttttcattctcaattgctttgtgtgataaaaaactaaagagacttctggtccaatttctggcaacatcccttctgaaaggtgagtagagtgggtgtcttctatgcccattttccccaattttacacaaactattatcaatgaacttttaagtacctagaatgggtaaaaccagagcaagactttaaattaccttcttctttcttctactggcagttctgcctccatcactatcaggctagggtgaccttcccttggtcaagccccaattgcccatgatttgtgcctgtgccctttctccagtgaccatttggtgaccagatggtagatatagaaaggggatggcatttgcaagtgactagtctgccacaaaatgctcatctgattagccactgctgccctggcaatggctttgtaagagtcaatgagaactagagccaggctgtggtccctggccatcaacagtgttggtgacggcagggagtccctttggtttaataaatccagtttttctttgggtatccaaattctcccctccttttgtaggagtcaggctctcagaacctgtgtccatgttggaacttcccccagtgtggatgcagatacgcagctcctgagctccagcctaaagtcttctgtagcctcagcaatacttgggcacctgctgtctcactgaatagctttcttttgtgacaaaggccacagacagcccttagactattccggaaacagtaggaaaaattacatatgtctttgacttctttattctgactccactgattttagccataatactttaaggagctactttttactaccccttaccgtgctgacttctgcaggtctgccctgtgacctgtcaggaactcctgagttacgctactggggtcacctgttgctcccctagcaagttaggcatgtcatatatttttaacagctttattgagatataattcacatattacacaattcacctttaaaacatacgattcaatggttttcagcaaactcacagagttgtccgcccacttgagagcaaacacatgttcaattttcttttcctttttttttttgagacagagtcagctttgttgcccaggctggagtgcagtgccatgatcttggctcactgcagcctccccatcctgggttcaagtgatccttctgcttcagcctccccagtagctgggattacaagcatgcgccaccacgcctagctaatttttgtgtttttagtagagatggggtttcaccatgttggccaggctggtctcaaactcctggactcaagtgatccacccacctcggcctcccaaagtgctgggattacaggtgtaagccaccgtgcctggcctacatgttcaattttctatgaacaaaggctttagtccttgacccagggctaaagtggtctgtccaagctgttgttggtagagggagtatgataaaatgtttaaatctcatttggttaccttgagtcctggaacatgcagtaactgtcatgctatagacatcatctgtatttggctgggaatacaaatgaagattgtggtgtattcaagcagtagggtttttgcttttgtttttgttttagtgccaacaaaacttttttttgtctgactacattaaagataagactgactatatttatacaacagaaactttgtaatagattttttcagctttgtgaaatcgaattttttttcatcagggctggttggatttcctttttaccctgtaatccaagcgttaatagtttgttagaagatgggttattgcatgtcactttttttttttgtaaaataaaaacataccttacaaaaaaaaaaaaaaaaaaaaaaaa

SEQ ID NO: 18 - DNA, 2393 bp, Homo sapiens
gctggctcagggagcgcacttccttctcagccagcgcgtcgccccctgcatccgtggcctccactggagctgggcaggaccctacccagtgaatctggagaaaacaaacttgggagacagacgaaagcttagggcacattggaggacagcgcagctgtggctcccatttttggagatgcagtcgaatttgagctcacagggaggtgtggttgcctcctggggatggaaaggcttcctttctccacctctgtaactggtgcttctgagaagtaaatggtatttggatcctgacctcagacgcgaatttgggtcttctgtgcttaggagcagaaagagcccaggaggggcctgttcctttacttcttgggggaaacgcaatgcgtggcctgacttctcatgacgggaaaggctactccaccttctctgtactcctggaggggagtcttgttcacatgtttaccagcggccaggacaaggaagagaaaagaaatgagccgtcagactgctacagcattacctaccggtacctcgaagtgtccaccatcccagagggtgcctgccctgactggcacaactgcatccaacaatgacttggcgagtctttttgagtgtccagtctgctttgactatgtgttaccgcccattcttcaatgtcagagtggccatcttgtttgtagcaactgtcgcccaaagctcacatgttgtccaacttgccggggccctttgggatccattcgcaacttggctatggagaaagtggctaattcagtacttttcccctgtaaatatgcgtcttctggatgtgaaataactctgccacacacagaaaaagcagaccatgaagagctctgtgagtttaggccttattcctgtccgtgccctggtgcttcctgtaaatggcaaggctctctggatgctgtaatgccccatctgatgcatcagcataagtccattacaaccctacagggagaggatatagtttttcttgctacagacattaatcttcctggtgctgttgactgggtgatgatgcagtcctgttttggctttcacttcatgttagtcttagagaaacaggaaaaatacgatggtcaccagcagttcttcgcaatcgtacagctgataggaacacgcaagcaagctgaaaattttgcttaccgacttgagctaaatggtcataggcgacgattgacttgggaagcgactcctcgatctattcatgaaggaattgcaacagccattatgaatagcgactgtctagtctttgacaccagcattgcacagctttttgcagaaaatggcaatttaggcatcaatgtaactatttccatgtgttgaaatggcaatcaaacattttctggccagtgtttaaaacttcagtttcacagaaaataaggcacccatctgtctgccaacctaaaactctttcggtaggtggaagctagacacatgaaggtaaataaaaagaaaggctgttaaatacaggaaacagttgcatgtagtaacactaatatatttaaaaataagtcaacagtaaaccactgaaaaaatatatgtatatacacccaagatgggcatcttttgtattaagaaaggaagcattgtaaaataattctgagttttgtgtttgttgtagattgattgtattgttgaaaaagtttgtttttgcgtgggagtgtgtgcctgcgtgggtgtgtgcgtgtttgggtttttttcctttaactgacaagccatcttgagtggtcatgggccactgcttttccctttgtgagtcaatacatagtgctgctgtgtgctttttttgtgtgtatttgctaatttttattaattttagtttttcattaaataaatttgacttttctgtaattcaggtttttcctttttttgtaccattttaaagttagtatcttttgatatgcatatttgtttatggtaaaaaatttataacgtgttcaatattttcttttcccccattaatcagttcattagaaatattttaaaatcagctattttgtgaagccatgagttccagaaagtaaaggtgacatcggaaaaataatcaaaagctatttaaagcatctataaggtgctctctttctgtcttctacagatgagtcacacctttgagcttaatctttgaaaggttagagaataaattgatttttataaatactgcaaatcaggcttttgtttcctttttcagatatcttggacaaatcacatattttaaaatttgttcttgtatttattggttttgcagaagaaggcatcgtcatgcacagtatttgtaattaaaagcaaatcatttgtttaaaaaggcagtttgcaaaaaatgtttttggtcttttataattctcattaaaagaatatctggcaaattaaaaaaaaaaaaa

SEQ ID NO: 19 - DNA, 818 bp, Homo sapiens
cgcagcgggtcctctctatctagctccagcctctcgcctgcgccccactccccgcgtcccgcgtcctagccgaccatggccgggcccctgcgcgccccgctgctcctgctggccatcctggccgtggccctggccgtgagccccgcggccggctccagtcccggcaagccgccgcgcctggtgggaggccccatggacgccagcgtggaggaggagggtgtgcggcgtgcactggactttgccgtcggcgagtacaacaaagccagcaacgacatgtaccacagccgcgcgctgcaggtggtgcgcgcccgcaagcagatcgtagctggggtgaactacttcttggacgtggagctgggccgaaccacgtgtaccaagacccagcccaacttggacaactgccccttccatgaccagccacatctgaaaaggaaagcattctgctctttccagatctacgctgtgccttggcagggcacaatgaccttgtcgaaatccacctgtcaggacgcctaggggtctgtaccgggctggcctgtgcctatcacctcttatgcacacctcccaccccctgtattcccacccctggactggtggcccctgccttggggaaggtctccccatgtgcctgcaccaggagacagacagagaaggcagcaggcggcctttgttgctcagcaaggggctctgccctccctccttccttcttgcttctcatagccccggtgtgcggtgcatacacccccacctcctgcaataaaatagtagcatcggcaaaaaaaaaaaaaaaaaaaaaaaaaaaaaaaaaaaaaaaaaaa

SEQ ID NO: 20 - DNA, 4248 bp, Homo sapiens
gggccatttctggacaacagctgctattttcacttgagccgaagttaatttctcggggagttctcgggcgcgcacaggcagctcggtttgccctgcgattgagctgcgggtcgcggccggcgccggcctctccaatggcaaatgtgtgtggctggaggcgagcgcgaggctttcggcaaaggcagtcgagtgtttgcagaccggggcgagtcctgtgaaagcagataaaagaaaacatttattaacgtgtcattacgaggggagcgcccggccggggctgtcgcactccccgcggaacatttggctccctccagctccgagagaggagaagaagaaagcggaaaagaggcagattcacgtcgtttccagccaagtggacctgatcgatggccctcctgaatttatcacgatatttgatttattagcgatgccccctggtttgtgtgttacgcacacacacgtgcacacaaggctctggctcgcttccctccctcgtttccagctcctgggcgaatcccacatctgtttcaactctccgccgagggcgagcaggagcgagagtgtgtcgaatctgcgagtgaagagggacgagggaaaagaaacaaagccacagacgcaacttgagactcccgcatcccaaaagaagcaccagatcagcaaaaaaagaagatgggccccccgagcctcgtgctgtgcttgctgtccgcaactgtgttctccctgctgggtggaagctcggccttcctgtcgcaccaccgcctgaaaggcaggtttcagagggaccgcaggaacatccgccccaacatcatcctggtgctgacggacgaccaggatgtggagctgggttccatgcaggtgatgaacaagacccggcgcatcatggagcagggcggggcgcacttcatcaacgccttcgtgaccacacccatgtgctgcccctcacgctcctccatcctcactggcaagtacgtccacaaccacaacacctacaccaacaatgagaactgctcctcgccctcctggcaggcacagcacgagagccgcacctttgccgtgtacctcaatagcactggctaccggacagctttcttcgggaagtatcttaatgaatacaacggctcctacgtgccacccggctggaaggagtgggtcggactccttaaaaactcccgcttttataactacacgctgtgtcggaacggggtgaaagagaagcacggctccgactactccaaggattacctcacagacctcatcaccaatgacagcgtgagcttcttccgcacgtccaagaagatgtacccgcacaggccagtcctcatggtcatcagccatgcagccccccacggccctgaggattcagccccacaatattcacgcctcttcccaaacgcatctcagcacatcacgccgagctacaactacgcgcccaacccggacaaacactggatcatgcgctacacggggcccatgaagcccatccacatggaattcaccaacatgctccagcggaagcgcttgcagaccctcatgtcggtggacgactccatggagacgatttacaacatgctggttgagacgggcgagctggacaacacgtacatcgtatacaccgccgaccacggttaccacatcggccagtttggcctggtgaaagggaaatccatgccatatgagtttgacatcagggtcccgttctacgtgaggggccccaacgtggaagccggctgtctgaatccccacatcgtcctcaacattgacctggcccccaccatcctggacattgcaggcctggacatacctgcggatatggacgggaaatccatcctcaagctgctggacacggagcggccggtgaatcggtttcacttgaaaaagaagatgagggtctggcgggactccttcttggtggagagaggcaagctgctacacaagagagacaatgacaaggtggacgcccaggaggagaactttctgcccaagtaccagcgtgtgaaggacctgtgtcagcgtgctgagtaccagacggcgtgtgagcagctgggacagaagtggcagtgtgtggaggacgccacggggaagctgaagctgcataagtgcaagggccccatgcggctgggcggcagcagagccctctccaacctcgtgcccaagtactacgggcagggcagcgaggcctgcacctgtgacagcggggactacaagctcagcctggccggacgccggaaaaaactcttcaagaagaagtacaaggccagctatgtccgcagtcgctccatccgctcagtggccatcgaggtggacggcagggtgtaccacgtaggcctgggtgatgccgcccagccccgaaacctcaccaagcggcactggccaggggcccctgaggaccaagatgacaaggatggtggggacttcagtggcactggaggccttcccgactactcagccgccaaccccattaaagtgacacatcggtgctacatcctagagaacgacacagtccagtgtgacctggacctgtacaagtccctgcaggcctggaaagaccacaagctgcacatcgaccacgagattgaaaccctgcagaacaaaattaagaacctgagggaagtccgaggtcacctgaagaaaaagcggccagaagaatgtgactgtcacaaaatcagctaccacacccagcacaaaggccgcctcaagcacagaggctccagtctgcatcctttcaggaagggcctgcaagagaaggacaaggtgtggctgttgcgggagcagaagcgcaagaagaaactccgcaagctgctcaagcgcctgcagaacaacgacacgtgcagcatgccaggcctcacgtgcttcacccacgacaaccagcactggcagacggcgcctttctggacactggggcctttctgtgcctgcaccagcgccaacaataacacgtactggtgcatgaggaccatcaatgagactcacaatttcctcttctgtgaatttgcaactggcttcctagagtactttgatctcaacacagacccctaccagctgatgaatgcagtgaacacactggacagggatgtcctcaaccagctacacgtacagctcatggagctgaggagctgcaagggttacaagcagtgtaacccccggactcgaaacatggacctgggacttaaagatggaggaagctatgagcaatacaggcagtttcagcgtcgaaagtggccagaaatgaagagaccttcttccaaatcactgggacaactgtgggaaggctgggaaggttaagaaacaacagaggtggacctccaaaaacatagaggcatcacctgactgcacaggcaatgaaaaaccatgtgggtgatttccagcagacctgtggtattggccaggaggcctgagaaagcaagcacgcactctcagtcaacatgacagattctggaggataaccagcaggagcagagataacttcaggaagtccatttttgcccctgcttttgctttggattatacctcaccagctgcacaaaatgcattttttcgtatcaaaaagtcaccactaaccctcccccagaagctcacaaaggaaaacggagagagcgagcgagagagatttccttggaaatttctcccaagggcgaaagtcattggaatttttaaatcataggggaaaagcagtcctgttctaaatcctcttattcttttggtttgtcacaaagaaggaactaagaagcaggacagaggcaacgtggagaggctgaaaacagtgcagagacgtttgacaatgagtcagtagcacaaaagagatgacatttacctagcactataaaccctggttgcctctgaagaaactgccttcattgtatatatgtgactatttacatgtaatcaacatgggaacttttaggggaacctaataagaaatcccaattttcaggagtggtggtgtcaataaacgctctgtggccagtgtaaaagaaaatccctcgcagttgtggacatttctgttcctgtccagataccatttctcctagtatttctttgttatgtcccagaactgatgttttttttttaaggtactgaaaagaaatgaagttgatgtatgtcccaagttttgatgaaactgtatttgtaaaaaaaattttgtagtttaagtattgtcatacagtgttcaaaaccccagccaatgaccagcagttggtatgaagaacctttgacattttgtaaaaggccatttcttgggaaaaaaaaaaa

SEQ ID NO: 21 - DNA, 3453 bp, Homo sapiens
cacggtgcctgcacgggacgccgctcgatccccgagccccctcgccgtccccgacagcccccccggcgccatgcaatcccggcttctactcctcggggcacccggaggccacggcggcccggcctcgcggcgcatgcggctgctcctgcggcaggtggtgcagcgcaggccgggtggcgacaggcagcggccggaggtcagactgttgcacgccggctcgggggccgacacaggtgatacagttaatattggagatgtatcctacaagttgaaaattcctaagaatccagaacttgtgccacagaactacatttcagactctctggctcaatctgtagttcagcatctaagatggataatgcagaaggatcttttggggcaagatgtttttctaataggacctcctgggcctcttcgacgctctattgctatgcagtacttggagctgaccaaacgggaggtcgaatacattgccctgtcaagggacaccactgaaactgatctcaaacagcgacgagagatccgtgcaggcacagccttttacattgatcagtgtgcagttcgtgcagccacagaaggcagaactctcattttggaaggtttggaaaaggcagagaggaatgttttgcctgttttgaacaacttgctggaaaacagagagatgcagcttgaagatggacgcttcctgatgtctgctgagcgttacgacaaacttctccgagatcataccaaaaaagagttggattcttggaaaattgtccgagttagtgaaaatttccgagtgattgccttgggcttgccagtgccaaggtattctgggaatccattagacccccctcttcgttctcgatttcaagccagggatatttattatttacccttcaaggaccaacttaagttgttatattcaattggagccaatgtttctgctgagaaagtttctcagctcttgtcctttgccacaactctgtgttcccaagaatcttctactcttggacttccagactttcctttagatagtttagcagctgcggttcaaatcttggattcctttcctatgatgccaatcaaacatgcaatccagtggctttatccatatagtattttactaggtcatgaagggaagatggctgtggaaggtgttttaaagcgctttgaacttcaagattcaggaagctctctacttcctaaagagattgtaaaagtagagaagatgatggaaaaccatgtgtcccaagcttctgtgaccatccggattgcagataaagaggtgaccattaaggtgccagccgggaccaggctattaagtcaaccttgtgcgtcagaccgtttcatacagactttgagccataagcagctacaggctgaaatgatgcagtctcacatggttaaagatatatgtttaattggaggaaagggttgtggaaaaacagtgatcgctaagaactttgccgataccttaggatacaacatagaacctattatgctctatcaggatatgacagcgcgtgatctgctacagcagagatacacccttccaaatggagacactgcctggcggtcctcaccccttgtgaatgctgctctggaaggcaagctggtcctgctggatggcattcaccgggtgaatgcgggcacgcttgctgtattgcaaaggttaatccatgatcgagagctaagcctctatgatggttctaggctgctgagagaagacaggtatatgcgtttaaaggaggagctgcaactgtctgatgaacagctacagaagagatccatttttcctatccatccctccttcagaatcattgccttggcagaaccccctgttattggaagcacagcacaccagtggctgggaccagaattcttaaccatgttctttttccattacatgaaaccacttgtgaaaagtgaagaaatccaagtgattaaggaaaaggtcccaaatgtacctcaggaagctctggataagttattatcatttacacacaaactcagagaaacacaggatccaacggcacaatcattagcggcatcactttctaccagacaactgttgcgaatttctcgtcggctgtcacagtatcctaatgaaaatcttcacagtgctgttactaaagcctgcctttccaggtttttacccagtcttgctaggtcagcattagaaaaaaatctggcagatgctacaatagaaataaatactgatgacaatttggagccagaactgaaggattacaaatgtgaagtaacatctggaactctgaggattggtgctgttagtgcaccgatctataatgcacatgagaaaatgaaagtgcctgatgttcttttctatgacaacattcagcatgtgatagtgatggaagatatgctgaaagactttctccttggagaacacttattattggttggcaaccagggtgtaggaaaaaacaagattgttgacagattccttcacctgctcaacagaccccgagaatatattcagctacacagggataccacagtacaaactcttacgcttcagccttcggttaaagacggacttattgtatatgaagactcacctttggttaaagcagtaaagttgggtcatattctggtagtagatgaggctgacaaagctccaacaaatgtcacgtgtattttaaaaactctagtagaaaatggagaaatgattctagcagatggaagacgcattgttgcaaattctgctaatgtgaatggaagagaaaatgttgtagtgattcatcctgattttaggatgattgttctggcaaatagacctggatttcctttcctaggcaatgatttcttcggtaccttaggtgatatttttagctgccatgcagttgataaccccaaaccccactcggagctcgagatgctcagacagtatggaccaaatgtgcctgagcccatccttcagaagcttgtggctgcctttggagagctgaggagtttggctgaccaagggattattaactatccttattctaccagagaagttgtcaacatagtcaaacatttacagaaatttccgactgaaggtctctccagtgtagttcgaaatgtgtttgactttgattcctacaacaatgacatgagggagatattgattaacactttacacaaatacgggatacctatcggagcaaagcctaccagtgtgcagctggcaaaggagtaaggcaaattcagtactcttctgattcttaaatgctgatagttcttgatttatcattccattctacaaaactgagttgctatggaaattactttccatgagtaagcttttcaaatacatgtatatgttgctgtttgctggctgcttttcagttaaatagcattatttataggcctgtaaattgaaaattttgattattatgtatgtcccatgagtagatgtaataaataaagggaaatatttgtttctttaaaaaaaaaaaaaaa

SEQ ID NO: 22 - DNA, 3074 bp, Homo sapiens
attgtcttccaggaaacagctccctcagtttggaatcagctctcccgctgcggccgcagtagccggagccggagccgcagccaccggtgccttcctttcccgccgccgcccagccgccgtccggcctccctcgggcccgagcgcagaccaggctccagccgcgcggcgccggcagcctcgcgctccctctcgggtctctctcgggcctcgggcaccgcgtcctgtggggcggccgcctgcctgcccgcccgcccgcagccccttcgctgcgcggcccctgggcggccgctgccatgggcaccgacagccgcgcggccaaggcgctcctggcgcgggcccgcaccctgcacctgcagacggggaacctgctgaactggggccgcctgcggaagaagtgcccgtccacgcacagcgaggagcttcatgattgtatccaaaaaaccttgaatgaatggagttcccaaatcaacccagatttggtcagggagtttccagatgtcttggaatgcactgtatctcatgcagtagaaaagataaatcctgatgaaagagaagaaatgaaagtttctgcaaaactgttcattgtagaatcaaactcttcatcatcaactagaagtgcagttgacatggcctgttcagtccttggagttgcacagctggattctgtgatcattgcttcacctcctattgaagatggagttaatctttccttggagcatttacagccttactgggaggaattagaaaacttagttcagagcaaaaagattgttgccataggtacctctgatctagacaaaacacagttggaacagctgtatcagtgggcacaggtaaaaccaaatagtaaccaagttaatcttgcctcctgctgtgtgatgccaccagatttgactgcatttgctaaacaatttgacatacagctgttgactcacaatgatccaaaagaactgctttctgaagcaagtttccaagaagctcttcaggaaagcattcctgacattcaagcgcacgagtgggtgccgctgtggctactgcggtattcggtcattgtgaaaagtagaggaattatcaaatcaaaaggctacattttacaagctaaaagaaggggttcttaactgacttaggagcataacttacctgtaatttccttcaatatgagagaaaattgagatgtgtaaaaatctagttactgcctgtaaatggtgtcattgaggcagatattctttcgtcatatttgacagtatgttgtctgtcaagttttaaatacttatcttgcctccatatcaatccattctcatgaacctctgtattgctttccttaaactattgttttctaattgaaattgtctataaagaaaatacttgcaatatatttttcctttatttttatgactaatataaatcaagaaaatttgttgttagatatattttggcctaggtatcagggtaatgtatatacatattttttatttccaaaaaaaattcattaattgcttcttaactcttattataaccaagcaatttaattacaattgttaaaactgaaatactggaagaagatatttttcctgtcattgatgagatatatcagagtaactggagtagctgggatttactagtagtgtaaataaaattcactcttcaatacatgaatggaaacttaaatttttttttatgtgtccttgcttatagtttagctgtaataatttaaccttgtattcttgtgccatattctgtctttttattacttataaagacaaaccaaagtaaatctgaaaggagactagaagctttgaaattattgtttgggggttttataaaagcaactactgtcacctccatccagattcttttaaattattgatccatccatagtatatattgctactcattcaagaatcctcaataagtattgagtatttaccatatgttgggatactgtgggctctggagagaggagggggcaatagagctaggaattaagaatcagttgagtaaaatgtgtaatatttattccccattaataactgactaggaaggactaaaagccagaaaggggatgaaaaaaaaatccttaattcagggccgacattatctacttaaacaactttgagatatggtcttaattattttaaagcagaataatataattgaaagtttatagctaaaagagactatataggtcatttagtataattcttcattagtttacgaaccacaaaattgcaaataaataagctatgaactttgatgtacactataaatctccttaattctataaatttgtgtctgtaacctgaatagtttgaaaacttctttaaaaatctcttgtatttcatccgggcgcagtggctcacacctgtaatcccagcactttgggaggccgaggtgggcagatcacgaggtcaggagtttgagaccagcctgaccaacatggtaaaaccccatctctactaaaatacaaaaattggctgggcgtggtggcactcgcctgtaatctcagctacttgggaggctgaggcaggagaatcgcttgaacccgggaggcggaggttacagtgagccgagatcacatcactgcactccagcctgggcgacagagcgagactccatctcaaaaaaaaaaaaaaactcttgtatctcaatatttttaaaccacaggcctaaataaaactaattttgctcaagttttctcaacctagggaaaaagaactatggttccatattcaaaataaatattatagacccttttcctaagtaggattttgtggtttactgattgggtaatttgatcattaaaattatgtgaaatctgcccgggcacacctcatgcctgtaatcccagcactctgggaggccaaggcagatgatcacctgaggtcaggagttctagaccagcctggctaacatggtgaaaccctgtatctgctaaaaatacaaaaattagccaggcgtggtggcgggctcctgtaatcccagctactttggaggcgaggcacgagaatcgcttgaacctgggaggcggagtttgcagtgagccgagatcacgccattgcactccagcctgggcg

SEQ ID NO: 23 - DNA, 3036 bp, Homo sapiens
gggggagcccctgcaagtttcccgggccgcgcgccgcgctcgctcgcctcccagcccgcggcccgagccgccgccgcgcccgccatgccctcggccaaacaaaggggctccaagggcggccacggcgccgcgagcccctcggagaagggtgcccacccgtcgggcggcgcggatgacgtggcgaagaagccgccgccggcgccgcagcagccgccgccgccgcccgcgccgcacccgcagcagcacccgcagcagcacccgcagaaccaggcgcacggcaagggcggccaccgcggcggcggcggcggcggcggcaagtcctcctcctcctcctccgcctccgccgccgctgccgccgccgccgcctcgtcctcggcgtcctgctcgcgcaggctcggcagggcgctcaactttctcttctacctcgccctggtggcggcggccgctttctcgggctggtgcgtccaccacgtcctggaggaggtccagcaggtccggcgcagccaccaggacttctcccggcagagggaggagctgggccagggcttgcagggcgtcgagcagaaggtgcagtctttgcaagccacatttggaacttttgagtccatcttgagaagctcccaacataaacaagacctcacagagaaagctgtgaagcaaggggagagtgaggtcagccggatcagcgaagtgctgcagaaactccagaatgagattctcaaagacctctcggatgggatccatgtggtgaaggacgcccgggagcgggacttcacgtccctggagaacacggtggaggagcggctgacggagctcaccaaatccatcaacgacaacatcgccatcttcacagaagtccagaagaggagccagaaggagatcaatgacatgaaggcaaaggttgcctccctggaagaatctgaggggaacaagcaggatttgaaagccttaaaggaagctgtgaaggagatacagacctcagccaagtccagagagtgggacatggaggccctgagaagtacccttcagactatggagtctgacatctacaccgaggtccgcgagctggtgagcctcaagcaggagcagcaggctttcaaggaggcggccgacacggagcggctcgccctgcaggccctcacggagaagcttctcaggtctgaggagtccgtctcccgcctcccggaggagatccggagactggaggaagagctccgccagctgaagtccgattcccacgggccgaaggaggacggaggcttcagacactcggaagcctttgaggcactccagcaaaagagtcagggactggactccaggctccagcacgtggaggatggggtgctctccatgcaggtggcttctgcgcgccagaccgagagcctggagtccctcctgtccaagagccaggagcacgagcagcgcctggccgccctgcaggggcgcctggaaggcctcgggtcctcagaggcagaccaggatggcctggccagcacggtgaggagcctgggcgagacccagctggtgctctacggtgacgtggaggagctgaagaggagtgtgggcgagctccccagcaccgtggaatcactccagaaggtgcaggagcaggtgcacacgctgctcagtcaggaccaagcccaggccgcccgtctgcctcctcaggacttcctggacagactttcttctctagacaacctgaaagcctcagtcagccaagtggaggcggacttgaaaatgctcaggactgctgtggacagtttggttgcatactcggtcaaaatagaaaccaacgagaacaatctggaatcagccaagggtttactagatgacctgaggaatgatctggataggttgtttgtgaaagtggagaagattcacgaaaaggtctaaatgaattgcgtgtgcagggcgcggatttaaagtccaatttctcatgaccaaaaaatgtgtggttttttcccatgtgtcccctaccccccaatttcttgtcccctcttaaagagcagttgtcaccacctgaacaccaaggcattgtattttcatgcccagttaacttatttacaatatttaagttctctgcttctgcatttggttggtttcctgaagcgcagcccctgtgaataacaggtggcttttcatggatgtctctagtcagagaaaaatgataaaggcttaaattgaggattaacagaagcagattaacctcagaaatcctgtctggctggcagatttcaagtaaaaaaaaaaaaaaggtgggttggggggacccttttctttctagttgtctttaaggaaaattaattttacttttttttttgttctggccgaaatttttatgagatatctctcacttgtcttccactttgaaccggttaaagctcatagctgtcagctctgaatgaggaggggagaagcccctgggtctttctttgaaaggaatccgctgcttgagggctgcctccctcatggtgtgcgtgtcgtttctcttcctgacgcatcttgtgatatcagaggtaactatgcaaagcatccaggcggttctgaatgtgaagcactacacccagcagagtcccggtgccctctgtccccactgccggcccatgttctctctccggaggtcaccaaggaatgcacaggtttcgactaccagaaaggggagtccttgggttctttcaaaaaattcgtgaggagagctgtctacagtggaatagggggtctccctggggaatgcaggccaagtccttttattttaacatgatgtccatgaagaggtttgccgtctgggcagccctgtcggcaaggagcgtgcatactgcgtttgtgtaattgtttgctgtatctcccttccctctgagctgtattgttctttaatggctgtcttgcccttccaaaaaaaattgaaaaaaaaaaaaaaaaaaaaacccgaaaagtaaaaaaaaaaaaaaaaagttttgtttagtttactgtgaaatgaactgtatggcttatttacagtatttttaattcttaataaacacttgagctttgttatga

SEQ ID NO: 24 - DNA, 2968 bp, Homo sapiens
gaagtcttctgaagtgacaccagtgccttatccctagaggaatatcagcatagaaagcacagtcatgccaacagcgactagatagggaaatttccaactttctcaaaatgattaaggagtgtgacatggctaaagaaatgcttccatcatttcttcagaaagttgaagttgtctcagaagcttctagagaaacttgtgtagctttgactgattgccttaatctcttcaccaaacaagaaggggtgaggaattttaaattggaacaagagcaagaaaaaaacaaaatcttgtcagaagcactggagacgctggccactgaacatcatgaattagagcagtctctggtgaaaggctctccacccgccagcatccttagcgaggacgagttctatgatgcgctgtcagattccgagtccgaaaggtccctgagtagattggaagcagtgacagcacgctcctttgaagaggaaggagagcatttgggcagtagaaaacacagaatgtccgaagaaaaagactgtggtggcggagatgctctctccaatggcatcaagaaacacagaacaagtttgccttctcctatgttttccagaaatgacttcagtatctggagcatcctcagaaaatgtattggaatggaactatccaagatcacgatgccagttatatttaatgagcctctgagcttcctacagcgcctaactgaatacatggagcatacttacctcatccacaaggccagttcactctctgatcctgtggaaaggatgcagtgtgtagctgcgtttgctgtatctgctgttgcttctcagtgggaacggactggaaaacctttcaacccactgctgggagagacttatgaattagtgcgagatgaccttggatttagactcatctccgaacaggtcagccatcacccaccaatcagtgcatttcatgctgaaggattaaacaatgacttcatctttcatggctctatctatcccaaactgaaattctgggggaagagtgtagaagcagaacccaaaggaaccatcaccttggagctccttgaacacaatgaggcatatacatggacaaatcccacctgctgtgtgcataatatcattgtgggtaaactgtggatcgaacagtatggcaatgtggaaattataaaccacaagactggggacaaatgtgtgttgaattttaagccatgtggcctttttggtaaggaattacacaaagttgaaggctacattcaagataaaagcaaaaagaagctctgtgccctctatgggaagtggactgaatgtttatacagtgttgaccctgccacgtttgacgcttacaaaaaaaatgataagaaaaatacagaagagaagaagaacagcaaacagatgagcacctctgaggagttggatgaaatgccagtgccggattctgaaagtgtattcattatccctggaagcgttcttctatggcgaatagccccacggcctccaaattctgcccagatgtataattttactagttttgcaatggttttgaatgaagtagacaaagacatggagagtgtgattcccaagacagactgcaggttacggcctgacatcagagccatggaaaatggagagatagatcaagctagtgaagaaaaaaaacgacttgaggaaaaacaaagagcagcccgcaaaaacaggtccaagtcagaagaggactggaagacgaggtggttccatcaaggtcctaatccctacaatggagcacaggactggatttactctggcagctactgggacagaaattacttcaatttgcctgacatttattaaaatgcatacaagtcagggtgtttggctaatctacaaataagtcttaaacctatgtttttaaatttttttcccttggtttctacttatcttttaaaaaaaaaaatgaaaaaacactcatgagataactgcatttcacccaacaaaagcagggtataaggcgatattggtgatgaaagtcttaggaaaaatgcataattttgctataaaatgtacttatttggaatactattttatatagaggtaagagaacactgctggggaatatgctttttatggttgctgttgccatatttactgaaggtttatacctaaatgtaactttagctttatggaactatatagtaatcccaaatcaagttattttgaatatttttatgctgtcatgcttgaatgttttagatgtaacctttgacatatttagaactctcctcctatacaatgtttattctcagatatagaggttatgtcattttataaagacttcattgataagatggcttttattcatactaatcctcccaatgttaccccttccatcttccaagaagaaaaaaaatgcctgaatattcagaatagatatttctgatttgaaaattctaaagaattaaactggaaaagtatttcatttacttagtgctctgaatttacttttacagttttctgcagtcagtatcattaaaatggttaagtttacatttgaactgaaaatatgtataaaatctagcaattcacaaaaatgccctagaaatatagattttaatcaccattacataatgacaaaccttgttaaatgcttccacttccagtggcaaatgccactagggaaagtaagttgcactcatgtaagtatcaaactatataaaaggaggccttgtgcatttcaagtttgcaaagtacctgtgtacttaaaatatgtgtggagacctactgtacagtagttttgcccctttaattggggcacattcatcttaaatcttatagtatttatccacccaaaccccagactgagatactgctcccaggggcctaggtagctgccagtccgtgattttaattgctgtcttgaagttaacaagtgttataatgaaataatctacctgatgctaaataaaggctttagaatgttccccaaaaaaaaaaaaaaaaaaaaaaaa

SEQ ID NO: 25 - DNA, 6038 bp, Homo sapiens
taggttgtgacagttggaagtgtcatgtacaacatgcggcgattaagtctttcacccaccttttcaatgggatttcatctgttagttactgtgagtctcttattttcccatgtggaccatgtaattgctgagacagaaatggaaggagaaggaaatgaaactggtgaatgtactggatcatattactgtaagaaaggggtgattttgcccatttgggaaccccaagacccttcttttggggacaaaattgctagagctactgtgtattttgtggccatggtctacatgtttcttggagtctctatcatagctgatcggttcatgtcctctatagaagtcatcacatctcaagaaaaagaaataaccataaagaaacccaatggagagaccaccaagacaactgtgaggatctggaatgaaacagtttctaacctgaccttgatggccctgggatcttctgctcctgagattctcctttcagtaattgaagtgtgtggccataacttcactgcaggagacctcggtcctagcaccatcgtgggaagtgctgcattcaatatgttcatcattattgcactctgtgtttatgtggtgcctgacggagagacaaggaagattaagcatttgcgtgtcttctttgtgacagcagcctggagcatctttgcctacacctggctttacattattttgtctgtcatatctcctggtgttgtggaggtctgggaaggtttgcttactttcttcttctttcccatctgtgttgtgttcgcttgggtagcggataggagacttctgttttacaagtatgtctacaagaggtatcgagctggcaagcagagggggatgattattgaacatgaaggagacaggccatcttctaagactgaaattgaaatggacgggaaagtggtcaattctcatgttgaaaatttcttagatggtgctctggttctggaggtggatgagagggaccaagatgatgaagaagctaggcgagaaatggctaggattctgaaggaacttaagcagaagcatccagataaagaaatagagcaattaatagaattagctaactaccaagtcctaagtcagcagcaaaaaagtagagcattttatcgcattcaagctactcgcctcatgactggagctggcaacattttaaagaggcatgcagctgaccaagcaaggaaggctgtcagcatgcacgaggtcaacactgaagtgactgaaaatgaccctgttagtaagatcttctttgaacaagggacatatcagtgtctggagaactgtggtactgtggcccttaccattatccgcagaggtggtgatttgactaacactgtgtttgttgacttcagaacagaggatggcacagcaaatgctgggtctgattatgaatttactgaaggaactgtggtgtttaagcctggtgatacccagaaggaaatcagagtgggtatcatagatgatgatatctttgaggaggatgaaaatttccttgtgcatctcagcaatgtcaaagtatcttctgaagcttcagaagatggcatactggaagccaatcatgtttctacacttgcttgcctcggatctccctccactgccactgtaactatttttgatgatgaccacgcaggcatttttacttttgaggaacctgtgactcatgtgagtgagagcattggcatcatggaggtgaaagtattgagaacatctggagctcgaggaaatgttatcgttccatataaaaccatcgaagggactgccagaggtggaggggaggattttgaggacacttgtggagagctcgaattccagaatgatgaaattgtcaaaacaatatcagtcaaggtaattgatgatgaggagtatgagaaaaacaagaccttcttccttgagattggagagccccgcctggtggagatgagtgagaagaaagccctgttattgaatgagcttggtggcttcacaataacaggccaacctgtcttcaggaaggttcatgctagagaacatccgattctctctactgtaatcaccattgcagacgaatatgatgacaagcagccactgaccagcaaagaggaagaggagaggcgcattgcagaaatggggcgccccatcctgggagagcacaccaagttggaagtgatcattgaagaatcctatgaattcaagagtactgtggacaaactcattaagaagacaaacctggcccttgtggttgggactaacagctggagagaacagttcattgaagctatcactgtcagtgctggggaagatgatgacgacgatgaatgtggggaagagaagctgccctcctgtttcgattacgtgatgcactttctgactgtgttctggaaggtcctgtttgccttcgtcccccctactgaatactggaatggctgggcgtgtttcattgtctccatcctcatgattggcctactgacagctttcattggagacctggcttcccactttggctgcaccattggcctgaaagattctgtgactgcagtcgtgttcgtcgcacttggaacatcagtgccagacacatttgccagcaaagtggcagccacccaggaccagtatgcagacgcctccataggtaacgtcacgggcagcaacgcggtgaatgtcttcctgggaatcggtgtggcctggtccatcgctgccatctaccacgcagccaatggggaacagttcaaagtgtcccctggcacactagctttctctgtcactctcttcaccatttttgctttcatcaatgtgggggtgctgctgtatcggcggaggccagaaatcggaggtgagctgggtgggccccggactgccaagctcctcacatcctgcctctttgtgctcctatggctcttgtacattttcttctcctccctggaggcctactgccacataaaaggcttctaaaggaactatcagatatagtaaatttatatatatacatatatatacataaaaattatgtataatggacagaggaaactgacatttgtcatgttcacttacctgctgatggaatccagcttcaagagcatactctgtactagggccgaagtaaaaaaccatcacctcccattcccaggggcatcatcatgttcaacaaggcatggaggcagggccatctttgcagctcagtctagaagggctgcactctctccaggttgataaatccttaaggctttgatttgttttgtttttggttttgttttcagtggagctggggaggtagttaatgtttggctttatttttgttattttgttttgttttgtttttttgggagagtcagggttgttgcttttctttgtggaaagtgaaaccatccaaatgtaaatgggttttggtaaaaatttaaatcattagtattccccctcacctcccccaatcactttaaaacttattttggattaagaaaaaatctgggcatggaagaagaaagaagcatgtcttcatcgtattaccaaagttcatgcttatctccggaatgtgagtggaggtgaagctgcctccaagaagaagcataaaagtggaatggagccaggaaatccgatggttctagaaatagtctgatatttaaacatgtgatacctggcagtctcgtttaacaggtacaaggaaaacgtgcctagattcccaggaacgtgcaaaatcctttctttcttatctctttagctctggactgtgattggcaaggtccttcttccagcattcagcccagctaagcccccaggtgccccatcccaaccctgttcctcctgtccacctgccatcccctatgcaaacagtaagaataaccccattcaaaaagcacatcatcgttttccatttgcattaacatgtgtctcagtcccatgttgccgttgcttgggattgtctgtcagttttattttcaaaggcatccatggcttgcacaatcctgttccagtcatgactgaacatttgctccttcttcatgtgccgttcggaaatgttgttgtgatacctgttacacagtgcatggtgaaaaacaaataaaacaaaacaaagagtatctgtatatagtagagtatagtacatactgttctcccatttggcaatgttgattggacattgaagacataagtgagttttcttttcacctgagttgttacttttgtgctgttattgagtttgattaattactagggataaaaggagaaaatggattattgttcacggttctgcacattcatttctaagaagcaataactgtcatgtggggagaagttaaagctattgagaggatagcaggcaaactacaaagatcttcatggaaaattagccatgtggaacacatcagaggcctctaaaaatcacccattaattcaggaaggccaaggagaaaggccttatagagacgttgatatgttggatgtgcctaggctttcagagccaccctttccacaacacccctccctgcaaagtatttatttcacatctgcactgtctggcacagatggtagatagtgctggtttgttcattttatttttttacttaaaaggctattttgagccctgtttctttcactgtccagtctagtcctctttgattatatcagtagttgctgagtaagaaagaagccagggtgaccaacgggcctttaaaagtgttgtctcctctacttatgctgaaagagaaggcaattaaataagactagtacctcccaggaggattggactgggatatttttaaccctttaaaaagaatagctgtttctatgttaaaataccaaagaacatggataaacccaacattccaaagtagtgagtccactaatgagaaaaataatagaatgactttggtcacctctcggagacttctgtgtctataaggaatcccaggctggagacattcctagccctctgtattgattcaaaaatacttaataaattaaagctgttgagacttattttttcttctgtcactcagtaaccatgatccttcctcatttaataaacatttggtgactgaatgagtaattaaatgctggttacccacttaatgtgccaggtagtatgatactttctggggactaagccatgaacaaaacagtctagatccctgccctcagaagggttacagtttatgtgattactattttcatgagtaaaagtgaagaaagccatatggaaagatttttatcttgcaagaaaaaacaattatgaaactcttttaacataaacacactgaaactgtatcaaagcaattgtccaaattgtatttatacccaagaatttctttaactaagagagcataaggcatatgtttggaaaaccaccctctttatctttgaccgacttgcagataaatatatctctccattttaaaccaagaagggcaatcatgttggtgatccagatcactgagaaagcccagtgtatcccatcttttatctttgttggcaatggaacttttctatggcccacactttacaattctttgtcattctaacccatccttcccatccttattttttttttttttgagaattgctaaatggaaagctagcctagaagcaccaagtaaatatattcaaggaatataagttgtttaaacattagaaaaatttttgcactcattttttagctgtattaggaatgtcaataatcctgtagcaaattttcacagagaactttaagaaattcttgcattggtcgatttcaatttgaaagctttttggtttgtttgctttttaaattttcatgttctaggaaactatgattctggttgttcaggattgttattattatagttgtgtaaaattattttattttgtgtgtattgtgcacagcttggggggggggcgggaaatgcactaattgtgctcttccttataaatggtacatattactgacacagacaaataaagtttctaattgtttctgatttaatcactagtgatacagcatattctgtatgaaatgttttctcctttctcattgtcatctacttcattttttgttttcatgttttgaagaaataaaaaccaaaatggta

SEQ ID NO: 26 - DNA, 2700 bp, Homo sapiens
aggaccggaggcgcgacccctcggcccaccttgaggcccggcctaggactggaggcgcgacccctcggcccaccttgaggcccggcctaggaccggaggcgcgacccctgggcccaccttgaggcccggcctaggactggaggcgcgacccctcggcccaccttgaggcccggcctaggaccggaggcgcgacccctgggcccacctggcggcccggccggcacagcccgcctctcctctccaggccagggccccacaccttccgcctgacagccagccaagctcttcagtcccccgccctccacctgccagggaggctccgggcgttgtacctgccaccacggggtaggtctggtacaaagcactgggaatacaaccttgaacaagacaagaatcctggccttcaaggaacttcgaaccttttgggggatatgataagacacattaggaatcaccatgttaatggttatctttcttgaaggatataagtccagacacatatactcacagaaatgagttatttaaagaagacggtccagattccacagtccaaaaaaaaaagaggtttgccctgcttctgaaggagtcagggtatgaatgcagaggtcttcctgttggctcacatatcagatcttcaacgtcaacattaaatgcgagtgatgcttccccctctgtgaatgtggctttgggtatggctaatccacaccctgcactctggccttcagaagcccagggagagaagcctcccagaagcaaccttccagaaccttctccatcctcccaccgacctccccagcccctgtcctctctttgaatctcgttgtcaagttcttcccgcagacacctggctccttgaggggaggtgcagctttcatctgaccatgcaggcgtgctttgcagtgggtagagcagtgctttcctcctcccagctgcacactggaatcacctggagagtccagaaacttcccgcctcagtgaaagaacatcagtgtatcagcacagcaaatattccaaatgccaggctggattccctccaattaccaggacctccaggcttctcctcctttcaagaactttctgaccctggatccagtctaaatgttggttataaactcacctgacatccacagggcccttccaaaatactgtcaaggctgactgctggtgcctggaacctgcacaccctcctgggactctgcatctctgctggggctggtttctcctctgagccaccttaaggaatcccaccctgacccggtcagagctcaccatgcctccctctcctacttgctcagcattttgtgtctatctagactgaagcctttgctttctctctgtgattgtggtgaaagttcagtttgctttcctatctctagaatgtcagcacgaaggcagaggcatggtctcctgagttccataaccccaaagcctaactccctgcctgccacactgaggggcttagtagacattgacttactaaattgaatagttgagtaataatgctctttgaaataatttggagttagatcttgagccttgtgtggccagaggatgggtgaggacacatcataaaaactaaaggtaactgagcatgccagtggttggggtgagaaatttgcatggagcactccagacacagaggcagcaaaacccaagtgctgggaagtcagagtgagagagtgaaggggcagtgagtgcttctttaggagctggatcaaaacctaagatgatctggccacatggtgcagactgagcacacacttgggagagaaatgggccaggaggagaggagagaggttcaccacaggacagtggatgactgggaatagaagccctccccatggtggaccaagaagtctgtgaagctgagagaaagacaaaagaaatcacgacttctgtaggtagctgggatggaaacctcaggggtgacagggtctttaaaggaaatctcaaggaaggcatcatgttctgggtgtgctccagggatgctgagtaagaaaagaccagcctttctgtgtgtgtctttactctgccacaaggcacctgggctcctaaatgaccaatacacatgtgctatactggtgccaagaaagaagggtgttttgtaggaaagtcaaggtgagaggaggtgcccaggacttggctcctttctttgaagggtgacggtgtaaattctccatgctggggatcctactgatggatggaggcatgtcgatctcatcatttcacagagtggggagctatggccacaacgtgcctatcacacaagtccagcctcacgtcagctggacaccagctgttctgacacttgtcagcacatggggcacccggaagttcatggtgttctgcaaagaacgccacaaaacagtgagctcaacttcttgaaagtcataatgtgttgctaagtccgatcccagatcctaaaaataatatgaggtagtggccattgaacagaaaaaatttacatcttaaacttgaagataagtacaagtgtacctttttctaccactgttgttcagttttattatattgacaagcacctctgcccaccctgtggggtaaaactgtttgaaagtgtttatgaaagttttatataaatattggttgatcaaatgtacaaaatatgcatgatggcaccatattgtgtataatagtgacaaatacagtctatgtgggtggagaaacaagctcggtgttgtg

SEQ ID NO: 27 - DNA, 1475 bp, Homo sapiens
ggaaaaaaaagatgcgccctctgtcactgagggttgactgactggagagctcaagtgcagcaaagagaagtgtcagagcatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattttttataataaactctttataacaagattaaaa

SEQ ID NO: 28 - DNA, 840 bp, Homo sapiens
actcgccacctcctcttccacccctgccaggcccagcagccaccacagcgcctgcttcctcggccctgaaatcatgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagctgccgcaccagcccgaacaccattgagggagctgggagaccctccccacagtgccacccatgcagctgctccccaggccaccccgctgatggagccccaccttgtctgctaaataaacatgtgccctcaggccaaaaaaaaaaaaaaaaaa

SEQ ID NO: 29 - DNA, 2231 bp, Homo sapiens
aaaacaggaaataggtgtttcatatatacggctctaaccttctctctctgcaccttccttctgtcaatagatgaaacaaatacttcatcctgctctggaaaccactgatccctgttccaccggttttgttttcccagcaatgacattattcccagtgctgttgttcctggttgctgggctgcttccatcttttccagcaaatgaagataaggatcccgcttttactgctttgttaaccacccaaacacaagtgcaaagggagattgtgaataagcacaatgaactgaggagagcagtatctccccctgccagaaacatgctgaagatggaatggaacaaagaggctgcagcaaatgcccaaaagtgggcaaaccagtgcaattacagacacagtaacccaaaggatcgaatgacaagtctaaaatgtggtgagaatctctacatgtcaagtgcctccagctcatggtcacaagcaatccaaagctggtttgatgagtacaatgattttgactttggtgtagggccaaagactcccaacgcagtggttggacattatacacaggttgtttggtactcttcatacctcgttggatgtggaaatgcctactgtcccaatcaaaaagttctaaaatactactatgtttgccaatattgtcctgctggtaattgggctaatagactatatgtcccttatgaacaaggagcaccttgtgccagttgcccagataactgtgacgatggactatgcaccaatggttgcaagtacgaagatctctatagtaactgtaaaagtttgaagctcacattaacctgtaaacatcagttggtcagggacagttgcaaggcctcctgcaattgttcaaacagcatttattaaatacgcattacacaccgagtagggctatgtagagaggagtcagattatctacttagatttggcatctacttagatttaacatatactagctgagaaattgtaggcatgtttgatacacatttgatttcaaatgtttttcttctggatctgctttttattttacaaaaatatttttcatacaaatggttaaaaagaaacaaaatctataacaacaactttggatttttatatataaactttgtgatttaaatttactgaatttaattagggtgaaaattttgaaagttgtattctcatatgactaagttcactaaaaccctggattgaaagtgaaaattatgttcctagaacaaaatgtacaaaaagaacaatataattttcacatgaacccttggctgtagttgcctttcctagctccactctaaggctaagcatcttcaaagacgttttcccatatgctgtcttaattcttttcactcattcacccttcttcccaatcatctggctggcatcctcacaattgagttgaagctgttcctcctaaaacaatcctgacttttattttgccaaaatcaatacaatcctttgaattttttatctgcataaattttacagtagaatatgatcaaaccttcatttttaaacctctcttctctttgacaaaacttccttaaaaaagaatacaagataatataggtaaataccctccactcaaggaggtagaactcagtcctctcccttgtgagtcttcactaaaatcagtgactcacttccaaagagtggagtatggaaagggaaacatagtaactttacaggggagaaaaatgacaaatgacgtcttcaccaagtgatcaaaattaacgtcaccagtgataagtcattcagatttgttctagataatctttctaaaaattcataatcccaatctaattatgagctaaaacatccagcaaactcaagttgaaggacattctacaaaatatccctggggtattttagagtattcctcaaaactgtaaaaatcatggaaaataagggaatcctgagaaacaatcacagaccacatgagactaaggagacatgtgagccaaatgcaatgtgcttcttggatcagatcctggaacagaaaaagatcagtaatgaaaaaactgatgaagtctgaatagaatctggagtatttttaacagtagtgttgatttcttaatcttgataaatatagcagggtaatgtaagatgataacgttagagaaactgaaactgggtgagggctatctaggaattctctgtactatcttaccaaattttcggtaagtctaagaaagcaatgcaaaataaaaagtgtcttgaaaaaaaa

SEQ ID NO: 30 - DNA, 2537 bp, Homo sapiens
gtccaggattctggctcagagttgcaccactgggttttatattcacttggatctttagttgttttggcgcctactgaggtctgaagtttgaatcctgcagtcaattgggatggtggcttgtaccccaaagtgccattgcaacccttgtccttcctgaggaaagggtggcagttgccctgtggaattcctgccctgctccccgtgggtgtccaggctgacagaagttgggactgtgtctggctggccgtaggaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatggaggatatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgaggcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaaaaccgaagaagatggcccagctccccaagaaagcctcagccattcactgcccccagctcttctccccaggtgtgttggggccttggcctcccctgctgaaggtggggattgcccatccatctgcttacaattccctgctgtcgtcttagcaagaagtaaaatgagaaattttgttgatattctctccttaaaaaaaaaaaaaaaaaaaaaaaaaaaaaa

SEQ ID NO: 31 - DNA, 2935 bp, Homo sapiens
ttttcctacatgctggccatggggaaatcaccactgggcactataagaagcccctgggctctctgcagagccagcggctccagctaagaggacaagatgaggcccggcctctcatttctcctagcccttctgttcttccttggccaagctgcaggggatttgggggatgtgggacctccaattcccagccccggcttcagctctttcccaggtgttgactccagctccagcttcagctccagctccaggtcgggctccagctccagccgcagcttaggcagcggaggttctgtgtcccagttgttttccaatttcaccggctccgtggatgaccgtgggacctgccagtgctctgtttccctgccagacaccacctttcccgtggacagagtggaacgcttggaattcacagctcatgttctttctcagaagtttgagaaagaactttccaaagtgagggaatatgtccaattaattagtgtgtatgaaaagaaactgttaaacctaactgtccgaattgacatcatggagaaggataccatttcttacactgaactggacttcgagctgatcaaggtagaagtgaaggagatggaaaaactggtcatacagctgaaggagagttttggtggaagctcagaaattgttgaccagctggaggtggagataagaaatatgactctcttggtagagaagcttgagacactagacaaaaacaatgtccttgccattcgccgagaaatcgtggctctgaagaccaagctgaaagagtgtgaggcctctaaagatcaaaacacccctgtcgtccaccctcctcccactccagggagctgtggtcatggtggtgtggtgaacatcagcaaaccgtctgtggttcagctcaactggagagggttttcttatctatatggtgcttggggtagggattactctccccagcatccaaacaaaggactgtattgggtggcgccattgaatacagatgggagactgttggagtattatagactgtacaacacactggatgatttgctattgtatataaatgctcgagagttgcggatcacctatggccaaggtagtggtacagcagtttacaacaacaacatgtacgtcaacatgtacaacaccgggaatattgccagagttaacctgaccaccaacacgattgctgtgactcaaactctccctaatgctgcctataataaccgcttttcatatgctaatgttgcttggcaagatattgactttgctgtggatgagaatggattgtgggttatttattcaactgaagccagcactggtaacatggtgattagtaaactcaatgacaccacacttcaggtgctaaacacttggtataccaagcagtataaaccatctgcttctaacgccttcatggtatgtggggttctgtatgccacccgtactatgaacaccagaacagaagagattttttactattatgacacaaacacagggaaagagggcaaactagacattgtaatgcataagatgcaggaaaaagtgcagagcattaactataacccttttgaccagaaactttatgtctataacgatggttaccttctgaattatgatctttctgtcttgcagaagccccagtaagctgtttaggagttagggtgaaagagaaaatgtttgttgaaaaaatagtcttctccacttacttagatatctgcaggggtgtctaaaagtgtgttcattttgcagcaatgtttaggtgcatagttctaccacactagagatctaggacatttgtcttgatttggtgagttctcttgggaatcatctgcctcttcaggcgcattttgcaataaagtctgtctagggtgggattgtcagaggtctaggggcactgtgggcctagtgaagcctactgtgaggaggcttcactagaagccttaaattaggaattaaggaacttaaaactcagtatggcgtctagggattctttgtacaggaaatattgcccaatgactagtcctcatccatgtagcaccactaattcttccatgcctggaagaaacctggggacttagttaggtagattaatatctggagctcctcgagggaccaaatctccaacttttttttcccctcactagcacctggaatgatgctttgtatgtggcagataagtaaatttggcatgcttatatattctacatctgtaaagtgctgagttttatggagagaggcctttttatgcattaaattgtacatggcaaataaatcccagaaggatctgtagatgaggcacctgctttttcttttctctcattgtccaccttactaaaagtcagtagaatcttctacctcataacttccttccaaaggcagctcagaagattagaaccagacttactaaccaattccaccccccaccaacccccttctactgcctactttaaaaaaattaatagttttctatggaactgatctaagattagaaaaattaattttctttaatttcattatgaacttttatttacatgactctaagactataagaaaatctgatggcagtgacaaagtgctagcatttattgttatctaataaagaccttggagcatatgtgcaacttatgagtgtatcagttgttgcatgtaatttttgcctttgtttaagcctggaacttgtaagaaaatgaaaatttaatttttttttctaggacgagctatagaaaagctattgagagtatctagttaatcagtgcagtagttggaaaccttgctggtgtatgtgatgtgcttctgtgcttttgaatgactttatcatctagtctttgtctatttttcctttgatgttcaagtcctagtctataggattggcagtttaaatgctttactcccccttttaaaataaatgattaaaatgtgctttgaaaaaagtcaaaaaaaaaaaaaaaaa

SEQ ID NO: 32 - DNA, 3056 bp, Homo sapiens
gacacatgatgctgtgaacgtcagggtgctcgccagggaagggccctacccagagggacagaaagaaagccaggaggggtagagtttgaagagaagatcatgttctccctgaagacgcttccatttctgctcttactccatgtgcagatttccaaggcctttcctgtatcttctaaagagaaaaatacaaaaactgttcaggactacctggaaaagttctaccaattaccaagcaaccagtatcagtctacaaggaagaatggcactaatgtgatcgttgaaaagcttaaagaaatgcagcgattttttgggttgaatgtgacggggaagccaaatgaggaaactctggacatgatgaaaaagcctcgctgtggagtgcctgacagtggtggttttatgttaaccccaggaaaccccaagtgggaacgcactaacttgacctacaggattcgaaactataccccacagctgtcagaggctgaggtagaaagagctatcaaggatgcctttgaactctggagtgttgcatcacctctcatcttcaccaggatctcacagggagaggcagatatcaacattgctttttaccaaagagatcacggtgacaattctccatttgatggacccaatggaatccttgctcatgcctttcagccaggccaaggtattggaggagatgctcattttgatgccgaagaaacatggaccaacacctccgcaaattacaacttgtttcttgttgctgctcatgaatttggccattctttggggctcgctcactcctctgaccctggtgccttgatgtatcccaactatgctttcagggaaaccagcaactactcactccctcaagatgacatcgatggcattcaggccatctatggactttcaagcaaccctatccaacctactggaccaagcacacccaaaccctgtgaccccagtttgacatttgatgctatcaccacactccgtggagaaatacttttctttaaagacaggtacttctggagaaggcatcctcagctacaaagagtcgaaatgaattttatttctctattctggccatcccttccaactggtatacaggctgcttatgaagattttgacagagacctcattttcctatttaaaggcaaccaatactgggctctgagtggctatgatattctgcaaggttatcccaaggatatatcaaactatggcttccccagcagcgtccaagcaattgacgcagctgttttctacagaagtaaaacatacttctttgtaaatgaccaattctggagatatgataaccaaagacaattcatggagccaggttatcccaaaagcatatcaggtgcctttccaggaatagagagtaaagttgatgcagttttccagcaagaacatttcttccatgtcttcagtggaccaagatattacgcatttgatcttattgctcagagagttaccagagttgcaagaggcaataaatggcttaactgtagatatggctgaagcaaaatcaaatgtggctgtatccactttcagaatgttgaagggaagttcagcaagcattttcgttacattgtgtcctgcttatacttttctcaatattaagtcattgtttcccatcactgtatccattctacctgtcctccgtgaaaatatgtttggaatattccactatttgcagaggcttattcagttcttacacattccatcttacattagtgattccatcaaagagaaggaaagtaagcctttttgtcacctcaatatttactatttcaatacttacatatctgacttctaggatttattgttatattacttgcctatctgacttcatacatccctcagtttcttaaaatgtcctatgtatatcttctacatgcaatttagaactagattttggttagaagtaaggattataaacaacctagacagtacccttggcctttacagaaaatatggtgctgttttctacccttggaaagaaatgtagatgatatgtttcgtgggttgaattgtgtcccccataaaagatatgttgaagttctaaccccaggtacccatgaatgtgagcttaccagggtctttgcagatgtaattagttaagttaaggtgagatcacactgaattagggtgggctctaaatccattatgactgttgttcttataagaagaagagaggcatagtcacctaggggaggaggccgtatgaagacagaggcagagattggagtgacgcatctccaagccaaggaattccaaggactgtaagccaccagtagaagctttgaagaggcaaggaaggattccctccaatagccttcaagtgtgaccctgctgacacctgcagaattcggacttctatcctccaaaaccgtgagggaataaatttcctttgttttaagccaccaactttgcaatactttgttacagcaaccctagacatgaggtactagacacagtacatctacacatatgaaaatgaatcaacacagaatgcagaagtagaacccttgctaaggactactgggcatcttcccaggacagcagccaaaagagaaccaccacttcctctcctgcctcctccttgctctctcctagagtccaaacccaaatgggccagttggatctgatgttcgtcagttctttacttctatttcctggggtactcaggagggcacacactatagataacttgggttagctgcataaaattcaatgtctcattaagttgcattaaactgagcttagatgtgtaagtttgctaacggatgggtttttttgttaagaactataggatttatgggaccaagtctagcgagtccagatatcaaaatcattataatgttatatttgctgttattagaatataatatagcttattatacaataaatatgtagactgtaaaatatatttctcactagtacctcctattttctttctctgttgaagtttttaaatcccacagataattaaattggcacctttatgcttgttcaaaaattaaaataatctattaaataagttcaaattaaagatttttacttcaaatgac

SEQ ID NO: 33 - DNA, 1193 bp, Homo sapiens
ggggggccatagttctccctgattgagacttgcctgctgctgtgaccactggtcttgtcctcttctccagcatggtgtgtctgaagctccctggaggctcctgtatggcagcgctgacagtgacattgacggtgctgagctccccactggctttggctggggacacccaaccacgtttcttggagcaggctaagtgtgagtgtcatttcctcaatgggacggagcgagtgtggaacctgatcagatacatctataaccaagaggagtacgcgcgctacaacagtgacctgggggagtaccaggcggtgacggagctggggcggcctgacgctgagtactggaacagccagaaggacctcctggagcggaggcgggccgaggtggacacctactgcagatacaactacggggttgtggagagcttcacagtgcagcggcgagtccaacctaaggtgactgtgtatccttcaaagacccagcccctgcagcaccacaacctcctggtctgctctgtgaatggtttctatccaggcagcattgaagtcaggtggttccggaacggccaggaagagaaggctggggtggtgtccacaggcctgatccagaatggagactggaccttccagaccctggtgatgctggaaacagttcctcggagtggagaggtttacacctgccaagtggagcatccaagcatgatgagccctctcacggtgcaatggagtgcacggtctgaatctgcacagagcaagatgctgagtggagtcgggggctttgtgctgggcctgctcttccttgggacagggctgttcatctacttcaggaatcagaaaggacactctggacttcagccaacaggactcttgagctgaagtgcagatgaccacattcaaggaagaaccttctgccccagctttgcaagatgaaaagctttcccacttggctcttattcttccacaagagctttgtcaggaccaggttgttactggttcagcaactctgcagaaaatgtcctcccttgtggcttccttagctcctgttcttggcctgaagcctcacagctttgatggcagtgcctcatcttcaacttttgtgcttccctttacctaaactgtcctgcctcccgtgcatctgtactccccttgtgccacacattgcattattaaatgtttctcaaacatggagttaaaaaa

SEQ ID NO: 34 - RNA, 20 nt, Homo sapiens
guccgcucggcgguggccca

SEQ ID NO: 35 - RNA, 22 nt, Homo sapiens
accuugccuugcugcccgggcc

SEQ ID NO: 36 - RNA, 22 nt, Homo sapiens
cguguauuugacaagcugaguu

SEQ ID NO: 37 - RNA, 22 nt, Homo sapiens
cgggguuuugagggcgagauga

SEQ ID NO: 38 - RNA, 23 nt, Homo sapiens
uguguacacacgugccaggcgcu

SEQ ID NO: 39 - RNA, 22 nt, Homo sapiens
cugggacaggaggaggaggcag

SEQ ID NO: 40 - RNA, 23 nt, Homo sapiens
ccugcagcgacuugauggcuucc